(19) United States
(12) Patent Application Publication
(10) Pub. No.: US 2026/0193716 A1
(43) Pub. Date: Jul. 9, 2026

(54) METHODS OF DETERMINING CANCER THERAPY EFFECTIVENESS

(71) Applicant: Strata Oncology, Inc., Ann Arbor, MI (US)

(72) Inventors: Daniel Reed Rhodes, Chelsea, MI (US); Scott Arthur Tomlins, Ann Arbor, MI (US); David Bryan Johnson, Chelsea, MI (US)

(21) Appl. No.: 19/131,165

(22) PCT Filed: Nov. 17, 2023
(86) PCT No.: PCT/US2023/080377

§ 371 Date: May 19, 2025

Related U.S. Application Data
(60) Provisional application No. 63/426,344, filed on Nov. 17, 2022.
(60) Provisional application No. 63/446,014, filed on Feb. 15, 2023.
(60) Provisional application No. 63/464,406, filed on May 5, 2023.

(51) Int. Cl. C12Q 1/6886; A61K 38/00; C07K 16/30; C12Q 1/6851; C12Q 1/686; C12Q 1/6869; G16B 25/10; G16H 10/40; G16H 10/60; G16H 20/10; G16H 50/30

(52) U.S. Cl. CPC C12Q 1/6886; A61K 38/00; C07K 16/30; C12Q 1/6851; C12Q 1/686; C12Q 1/6869; G16B 25/10; G16H 10/40; G16H 10/60; G16H 20/10; G16H 50/30

(57) ABSTRACT

Disclosed herein are methods for predicting if a cancer subject is likely to benefit from treatment with one or more antibody drug conjugates. Additionally, methods are disclosed herein for treating a subject with one or more antibody-drug conjugates determined likely to benefit from the one or more antibody drug conjugates.

### RELATED APPLICATIONS

This application claims the benefit of U.S. Provisional Application No. 63/426,344, filed on Nov. 17, 2022, U.S. Provisional Application No. 63/446,014, filed on Feb. 15, 2023, and U.S. Provisional Application No. 63/464,406, filed May 5, 2023. The entire teachings of the above applications are incorporated herein by reference.

### BACKGROUND OF THE INVENTION

Antibody-drug conjugates (ADC) represent an important new class of targeted cancer therapy for solid tumors, with several recent drug approvals and promising late-phase candidates¹. Approximately 80 ADCs are currently in various stages of development and with over 600 clinical trials ongoing, demonstrating a broad spectrum of objective response rates¹⁶. Despite variable response rates, most ADC development strategies have not utilized predictive biomarkers. In some cases, target protein expression has been used to select patients (e.g. Her2-targeted ADCs^{2,3}) or enrich clinical trial results (e.g. Folate receptor-targeted ADC⁴), yet correlation analysis conducted in recent studies of ADC treated metastatic triple-negative breast cancer patients¹⁹ and metastatic urothelial cancer patients²⁰ revealed that ADC target expression alone, whether measured via RNA or protein levels, is poorly correlated with objective response rates in the these ADC treated patients. Thus, to date, development strategies have pursued unselected patients in high unmet need tumor types known to express the target (e.g. metastatic bladder, triple-negative breast, ovarian)¹. Furthermore, recent clinical trials have documented toxicities associated with ADCs which are on par with toxicities of standard-of-care chemotherapies¹⁶.

Thus, there exists a need for a universal biomarker that is able to predict the effectiveness of available ADC therapies for an individual patient, thereby enhancing chances of successful treatment outcomes, while simultaneously reducing unnecessary patient exposure to toxicities associated with other ADCs which have been identified as less likely to provide a desirable treatment outcome. As described herein, the inventors have leveraged next-generation sequencing molecular data from 20,000+ subjects with advanced cancer and published objective response rates from 22 clinical trials and cohorts across 9 antibody-drug conjugate therapies, to develop a multivariate biomarker algorithm which is capable of identifying cancer patients that are likely to benefit from one or more ADC treatments, regardless of tumor type.

### SUMMARY OF THE INVENTION

In some aspects, the invention is directed to a method for identifying a subject as likely to respond to one or more antibody-drug conjugate (ADC) therapies, the method comprising the steps of: (A) measuring the expression level of at least one gene product associated with each of the one or more ADC therapies from a biological tissue sample obtained from the subject; (B) measuring in the same biological tissue sample of step (A), the expression level of one or both of: (i) at least one gene product associated with cell adhesion, and (ii) one or more gene products associated with proliferation, wherein if the expression level of the one or more gene products associated with proliferation is measured, calculating therefrom an average of all expression levels of the measured gene products associated with proliferation, thereby obtaining a proliferation gene expression level; (C) optionally, determining tumor cellularity in the same tumor tissue sample of steps (A) and (B); (D)(1) identifying a subject likely to respond to the one or more ADC therapies when one or more of a calculated ADC Treatment Response Score (ADC TRS) surpass one or more corresponding predetermined thresholds, wherein each of the one or more ADC TRS is determined from: (i) the measured expression level of the at least one gene product associated with a corresponding ADC therapy, and at least two of: (ii) the measured expression level of at least one gene product associated with cell adhesion, (iii) the determined proliferation gene expression level, and (iv) the determined tumor cellularity.

In some embodiments, the ADC TRS is determined by at least the combination of the expression level of the at least one gene product associated with a corresponding ADC therapy, the expression level of at least one gene product associated with cell adhesion, and the determined proliferation gene expression level. In some embodiments, both the determined proliferation gene expression level and the expression level of the at least one gene product associated with a corresponding ADC therapy are positively associated with a likelihood that a patient will respond to the corresponding ADC therapy, and the expression level of at least one gene product associated with cell adhesion is negatively associated with a likelihood that a patient will respond to the same corresponding ADC therapy.

In some embodiments, the ADC TRS is determined by at least the combination of the expression level of the at least one gene product associated with a corresponding ADC therapy, the measured expression level of at least one gene product associated with cell adhesion, and tumor cellularity. In some embodiments, both the tumor cellularity and the expression level of the at least one gene product associated with a corresponding ADC therapy are positively associated with a likelihood that a patient will respond to the corresponding ADC therapy, and the expression level of at least one gene product associated with cell adhesion is negatively associated with a likelihood that a patient will respond to the same corresponding ADC therapy.

In some embodiments, the ADC TRS is determined by at least the combination of the expression level of the at least one gene product associated with a corresponding ADC therapy, the expression level of at least one gene product associated with cell adhesion, the determined proliferation gene expression level, and tumor cellularity. In some embodiments, both the determined proliferation gene expression level, the tumor cellularity, and the expression level of the at least one gene product associated with a corresponding ADC therapy are positively associated with a likelihood that a patient will respond to the corresponding ADC therapy, and the expression level of at least one gene product associated with cell adhesion is negatively associated with a likelihood that a patient will respond to the same corresponding ADC therapy.

In some embodiments, the at least one gene product associated with each of the one or more ADC therapies comprise RNA transcripts individually selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5.

In some embodiments, the step of measuring the expression level of one or more gene products associated with proliferation comprises measuring the expression levels of a gene product of one or more genes selected from the group consisting of BIRC5, BRCA1, BRCA2, BUB1, BUB1B, CCNA2, CCNB1, CCNB2, CCNE1, CCNE2, CCNF, CCNG2, CDC2, CDC20, CDC25A, CDC25B, CDC25C, CDC45L, CDC6, CDC7, CDCKN1A p21, CDCKN3, CDK4, CDKN1C, CDKN2A, CDKN2C, CDKN2D p19, CDKN3, CENPA, CENPE, CENPF, CHAF1A, CHK1, CKS1, CKS2, CKS2, DHFR, DHFR, E2-EPF, E2F1, E2F3, E2F5, FEN1, FOXM1, KNSL2, KNSL5, KPNA2, LMNB2, MAD2, MAD2L1, MAPK13, MCM2, MCM3, MCM4, MCM5, MCM6, MK167, MNAT1, MSH2, MYBL2, NASP, NEK2, NPAT, ORC1, PA2G4, PCNA, PES1, PKMYT1, PLK, PRIM1, PTTG1, RAB6KIFL, RACGAP1, RAD51, RAD54L, RAN RAN, RFC4, RPA3, RRM1, RRM2, SLBP, STK12, STK15, STK18, STK6, TOP2A, TROAP, TUBB, TYMS, UBE2C, and UNG. In some embodiments, the step of measuring the expression level of one or more gene products associated with proliferation comprises measuring the expression level of one, two or all three of MYBL2, TOP2A, and/or UBE2C gene products.

In some embodiments, each of the gene products associated with cell adhesion impact at least two of an adherens junction, an anchoring junction, a cell-substrate adherens junction, a cell-substrate junction or a focal adhesion of a cell. In some embodiments, the step of measuring the expression level of at least one gene product associated with cell adhesion comprises measuring the expression level of a gene product of at least one gene selected from the group consisting of ATP2A2, BAIAP2, CD151, CHP1, CYFIP1, CYTH3, DAG1, DSC2, GIT1, HSP90B1, HSPA5, LIMK1, MAPK1, PACSIN2, PDIA3, PVR, REXO2, RPL22, RPLP1, RPLP2, RPS11, RPS16, RPS5, SDCBP SNAP23, SNTB1, and SRP68.

In one embodiment, the step of measuring the expression level of at least one gene product associated with cell adhesion comprises measuring the expression of a PVR gene product. In some embodiments, the step of measuring the expression level of at least one gene product associated with cell adhesion consists of measuring the expression level of a single gene product associated with cell adhesion. In some embodiments, the single gene product is a PVR gene product. In some embodiments, the single gene product is not a PVR gene product.

In some embodiments, measuring the expression level of at least one gene product associated with cell adhesion comprises measuring the expression level of two or more gene products associated with cell adhesion, and subsequently determining a cell adhesion gene product expression level by averaging the expression of two or more gene products associated with cell adhesion.

In some embodiments, the expression level of the at least one gene product associated with a corresponding ADC therapy is weighted by a factor of approximately 0.3 to 0.65, the expression level of at least one gene product associated with cell adhesion is weighted by a factor of approximately −0.8 to −1, and the determined proliferation gene expression level is weighted by a factor of approximately 0.2 to 0.4.

In some embodiments, the expression level of the at least one gene product associated with a corresponding ADC therapy is weighted by a factor of approximately 0.3, the expression level of at least one gene product associated with cell adhesion is weighted by a factor of approximately −0.9, and the tumor cellularity is weighted by a factor of approximately 0.8.

In some embodiments, the expression level of the at least one gene product associated with a corresponding ADC therapy is weighted by a factor of approximately 0.45, the expression level of at least one gene product associated with cell adhesion is weighted by a factor of approximately −1, the determined proliferation gene expression level is weighted by a factor of approximately 0.55, and the tumor cellularity is weighted by a factor of approximately 0.07.

In some embodiments, each of the one or more ADC TRS is determined by further taking into account a bias variable, which is a static offset tuned to yield biomarker frequencies that match published objective response rates in clinical trials. In some embodiments, the bias variable is weighted by a factor of approximately −0.25.

In some embodiments, the predetermined threshold is set to a percentile of ranked ADC Treatment Response Scores determined from tumor tissue samples of a first cohort of subjects utilizing at least the expression level of the at least one gene product associated with a first corresponding ADC therapy, wherein each percentile corresponds to a percentage greater than a percentage of subjects of the first cohort which do not respond to the same first corresponding ADC therapy.

In some embodiments, the predetermined thresholds are set to percentiles of ranked ADC Treatment Response Scores determined from tumor tissue samples of a first and a second cohort of subjects utilizing at least the expression level of at least a first and a second gene product associated with a first and second corresponding ADC therapy, wherein each percentile corresponds to a percentage greater than a percentage of subjects of the first and second cohort which do not respond to the at least first and second corresponding ADC therapies. In some embodiments, the predetermined threshold is zero, and an ADC TRS which indicates the subject is likely to respond to an ADC therapy is an ADC TRS with a value greater than zero.

In some embodiments, the first and/or second cohort of subjects is a pan-cancer cohort or a matched tumor-type cohort. In some embodiments, the first cohort of subjects and the second cohort of subjects are the same cohort of subjects.

In some embodiments, the expression level of the at least one gene product associated with each one or more ADC therapies, the expression level of at least one gene product associated with cell adhesion, and the tumor cellularity are log 2 transformed and/or Z score normalized prior to step (D)(1), and the expression level of the one or more gene products associated with proliferation are log 2 transformed and/or Z score normalized prior to the step of averaging expression levels of gene products associated with proliferation to obtain the proliferation gene expression level in step (B).

In some embodiments, the tumor cellularity is a molecularly determined tumor cellularity which is calculated based on somatic and germline variant allele frequencies and/or copy number profiles.

In some embodiments, the method further comprises measuring the expression level of at least one housekeeping gene selected from CIAO1, EIF2B1, and HMBS in the tumor tissue sample, and normalizing the expression levels of the at least one gene product associated with the one or more ADC therapies, the at least one gene product associated with cell adhesion and the one or more gene products associated with proliferation to the at least one housekeeping gene expression level to obtain normalized expression levels of the at least one gene product associated with each of the one or more ADC therapies, the at least one gene product associated with cell adhesion, and the one or more gene products associated with proliferation.

In some embodiments, the expression product of the at least one gene associated with each of the one or more ADC therapies, cell adhesion, and one or more genes associated with proliferation are individually selected from a ribonucleic acid (RNA) and a protein.

In some embodiments, the gene expression product of the at least one gene associated with each of the one or more ADC therapies, cell adhesion, and the one or more genes associated with proliferation are proteins, and measuring the expression level thereof requires making use of immunohistochemistry techniques.

In some embodiments, the gene expression product of the at least one genes associated with each of the one or more ADC therapies, cell adhesion, and the one or more genes associated with proliferation are RNA, and measuring the expression level thereof requires making use of RNA sequencing techniques.

In some embodiments, each of the one or more ADC therapies comprise a monoclonal antibody, at least one functional fragment thereof or a bispecific antibody which targets at least one epitope of at least one antigen selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5.

In some embodiments, the antibody comprises a bispecific antibody capable of targeting two epitopes of the same antigen or an epitope of two distinct antigens, wherein the same antigen or the two antigens are selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5.

In some embodiments, the antibody or fragment thereof is conjugated directly or indirectly to a cytotoxic drug.

In some embodiments, the cytotoxic drug is a DNA replication inhibitor selected from the group consisting of an alkylating agent, a DNA polymerase inhibitor, a nitrogen mustard and a topoisomerase inhibitor.

In some embodiments, the antibody, the at least one functional fragment thereof, or the bispecific antibody is fused to a protein which is toxic to a cancer cell. In some embodiments, the cytotoxic drug is a topoisomerase inhibitor.

In some embodiments, the subject has or is suspected of having a cancer not approved for labeled use of the one or more ADC therapies. In some embodiments, the tumor tissue sample is or is suspected of containing bladder cancer, salivary gland cancer, endometrial cancer, ovarian cancer, cervical cancer, head and neck cancer, non-melanoma skin cancer, thyroid cancer, cancer of unknown primary, cancer of the central or peripheral nervous system, neuroendocrine tumor, melanoma, esophagogastric cancer, small bowel cancer, sarcoma, hepatobiliary cancer, pancreatic cancer, gastrointestinal stromal tumor, renal cell carcinoma, glioma, appendiceal cancer breast cancer, small cell lung cancer, prostate cancer, esophagogastric cancer, non-small cell lung cancer, pancreatic cancer, lymphoma or colorectal cancer.

In some embodiments, the tumor tissue sample is a formalin-fixed paraffin-embedded (FFPE) tumor tissue sample. In some embodiments, the tumor tissue sample contains at least 20% tumor content.

In some embodiments, the method further comprises the step (E) administering the at least one of the one or more ADC therapies to a subject identified in step (D)(1) as likely to respond to the one or more ADC therapies.

In some embodiments, the each of the one or more ADC TRS is determined without taking into account tumor cellularity.

In other aspects, the invention is directed to a method for selecting one or more antibody-drug conjugate (ADC) therapies among two or more ADC therapies identified as most beneficial to treat a cancer in a subject, the method comprising the steps of (A) measuring the expression level of at least one gene product associated with each of the or more ADC therapies from a biological tissue sample obtained from the subject; (B) measuring in the same biological tissue sample of step (A), the expression level of one or both of: (i) at least one gene product associated with cell adhesion, and (ii) one or more gene products associated with proliferation, wherein if the expression level of more than one gene product associated with proliferation is measured, calculating therefrom an average of all expression levels of the measured gene products associated with proliferation, thereby obtaining a proliferation gene expression level; (C) optionally, determining tumor cellularity in the same tumor tissue sample of steps (A) and (B); (D)(1) calculating an ADC Treatment Response Score (ADC TRS) for each of the two or more ADC therapies, and determining each of the two or more ADC TRS surpass a predetermined threshold associated with beneficial patient treatment outcome, wherein each of the one or more ADC TRS is determined from: (i) the measured expression level of the at least one gene product associated with a corresponding ADC therapy, and at least two of: (ii) the measured expression level of at least one gene product associated with cell adhesion, (iii) the determined proliferation gene expression level, and (iv) the determined tumor cellularity; (D)(2) wherein if ADC TRS associated with two or more ADC therapies surpass a predetermined threshold associated with a beneficial patient treatment outcome, ranking the at least two ADC TRS by the value by which each ADC Treatment Response Score exceeds the predetermined threshold, and selecting the highest ranked ADC therapy for administration to a subject.

In some embodiments, the method further comprises step (E) of administering to the subject the selected highest ranked ADC therapy. In some embodiments, step (E) further comprises administering to the subject at least one other lower ranked ADC exceeding the predetermined threshold in combination with the highest ranked ADC therapy. In some embodiments, step (E) does not include administering another ADC therapy in combination with the highest ranked ADC therapy.

In other aspects, the invention is directed to a method for treating a cancer in a subject likely to respond to one or more antibody-drug conjugate (ADC) therapies, comprising: (a) measuring, in a tumor tissue sample obtained from the subject, the expression level of: i) at least one gene product associated with each corresponding one or more ADC therapies, and at least one of the expression levels of: ii) at least one gene product associated with cell adhesion, and iii) one or more gene products associated with proliferation; (b) measuring the expression levels of one or more housekeeping genes in the same tumor tissue sample of step (a) and further normalizing the expression level of the at least one gene product associated with each of the one or more ADC therapies, the at least one gene product associated with cell adhesion, and one or more gene products associated with proliferation of step (a) against the expression levels of the one or more housekeeping genes to obtain normalized expression levels of the at least one gene product associated with each corresponding one or more ADC therapies, a gene product associated with cell adhesion, and one or more genes associated with proliferation; (c) if gene products of one or more genes associated with proliferation are measured and normalized, determining proliferation gene expression level by averaging the normalized expression levels of the one or more gene products associated with proliferation; (d) optionally, determining tumor cellularity in the same tumor tissue sample of steps (a) and (b); (e)(1) identifying a subject likely to benefit from the one or more ADC therapies when one or more of a calculated ADC Treatment Response Score (ADC TRS) surpass one or more corresponding predetermined thresholds, wherein each of the one or more ADC TRS is determined from: (i) the measured expression level of the at least one gene product associated with a corresponding ADC therapy, and at least two of: (ii) the measured level of the at least one gene product associated with cell adhesion, (iii) the determined proliferation gene expression level, and (iv) the determined tumor cellularity; and (f) administering an effective amount of the one or more ADC therapies to a subject identified as likely to benefit from the one or more ADC therapies.

In some embodiments, the ADC TRS is determined by at least the combination of the expression level of the at least one gene product associated with a corresponding ADC therapy, the expression level of at least one gene product associated with cell adhesion, and the determined proliferation gene expression level. In some embodiments, both the determined proliferation gene expression level and the expression level of the at least one gene product associated with a corresponding ADC therapy are positively associated with a likelihood that a patient will respond to the corresponding ADC therapy, and the expression level of at least one gene product associated with cell adhesion is negatively associated with a likelihood that a patient will respond to the same corresponding ADC therapy.

In some embodiments, the ADC TRS is determined by at least the combination of the expression level of the at least one gene product associated with a corresponding ADC therapy, the measured expression level of at least one gene product associated with cell adhesion, and tumor cellularity. In some embodiments, both the tumor cellularity and the expression level of the at least one gene product associated with a corresponding ADC therapy are positively associated with a likelihood that a patient will respond to the corresponding ADC therapy, and the expression level of at least one gene product associated with cell adhesion is negatively associated with a likelihood that a patient will respond to the same corresponding ADC therapy.

In some embodiments, the ADC TRS is determined by at least the combination of the expression level of the at least one gene product associated with a corresponding ADC therapy, the expression level of at least one gene product associated with cell adhesion, the determined proliferation gene expression level, and tumor cellularity. In some embodiments, both the determined proliferation gene expression level, the tumor cellularity, and the expression level of the at least one gene product associated with a corresponding ADC therapy are positively associated with a likelihood that a patient will respond to the corresponding ADC therapy, and the expression level of at least one gene product associated with cell adhesion is negatively associated with a likelihood that a patient will respond to the same corresponding ADC therapy.

In some embodiments, the at least one gene product associated with each of the one or more ADC therapies comprise RNA transcripts individually selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5.

In some embodiments, the step of measuring the expression level of one or more gene products associated with proliferation comprises measuring the expression levels of a gene product of one or more genes selected from the group consisting of BIRC5, BRCA1, BRCA2, BUB1, BUB1B, CCNA2, CCNB1, CCNB2, CCNE1, CCNE2, CCNF, CCNG2, CDC2, CDC20, CDC25A, CDC25B, CDC25C, CDC45L, CDC6, CDC7, CDCKN1A p21, CDCKN3, CDK4, CDKN1C, CDKN2A, CDKN2C, CDKN2D p19, CDKN3, CENPA, CENPE, CENPF, CHAF1A, CHK1, CKS1, CKS2, CKS2, DHFR, DHFR, E2-EPF, E2F1, E2F3, E2F5, FEN1, FOXM1, KNSL2, KNSL5, KPNA2, LMNB2, MAD2, MAD2L1, MAPK13, MCM2, MCM3, MCM4, MCM5, MCM6, MK167, MNAT1, MSH2, MYBL2, NASP, NEK2, NPAT, ORC1, PA2G4, PCNA, PES1, PKMYT1, PLK, PRIM1, PTTG1, RAB6KIFL, RACGAP1, RAD51, RAD54L, RAN RAN, RFC4, RPA3, RRM1, RRM2, SLBP, STK12, STK15, STK18, STK6, TOP2A, TROAP, TUBB, TYMS, UBE2C, and UNG.

In some embodiments, the step of measuring the expression level of one or more gene products associated with proliferation comprises measuring the expression level of one, two or all three of MYBL2, TOP2A, and/or UBE2C gene products.

In some embodiments, each of the gene products associated with cell adhesion impact at least two of an adherens junction, an anchoring junction, a cell-substrate adherens junction, a cell-substrate junction or a focal adhesion of a cell. In some embodiments, the step of measuring the expression level of at least one gene product associated with cell adhesion comprises measuring the expression level of a gene product of at least one gene selected from the group consisting of ATP2A2, BAIAP2, CD151, CHP1, CYFIP1, CYTH3, DAG1, DSC2, GIT1, HSP90B1, HSPA5, LIMK1, MAPK1, PACSIN2, PDIA3, PVR, REXO2, RPL22, RPLP1, RPLP2, RPS11, RPS16, RPS5, SDCBP SNAP23, SNTB1, and SRP68.

In one embodiment, the step of measuring the expression level of at least one gene product associated with cell adhesion comprises measuring the expression of a PVR gene product. In some embodiments, the step of measuring the expression level of at least one gene product associated with cell adhesion consists of measuring the expression level of a single gene product associated with cell adhesion. In some embodiments, the single gene product is a PVR gene product. In some embodiments, the single gene product is not a PVR gene product.

In some embodiments, measuring the expression level of at least one gene product associated with cell adhesion comprises measuring the expression level of two or more gene products associated with cell adhesion, and subsequently determining a cell adhesion gene product expression level by averaging the expression of two or more gene products associated with cell adhesion.

In some embodiments, the expression level of the at least one gene product associated with a corresponding ADC therapy is weighted by a factor of approximately 0.3 to 0.65, the expression level of at least one gene product associated with cell adhesion is weighted by a factor of approximately −0.8 to −1, and the determined proliferation gene expression level is weighted by a factor of approximately 0.2 to 0.4.

In some embodiments, the expression level of the at least one gene product associated with a corresponding ADC therapy is weighted by a factor of approximately 0.3, the expression level of at least one gene product associated with cell adhesion is weighted by a factor of approximately −0.9, and the tumor cellularity is weighted by a factor of approximately 0.8.

In some embodiments, the expression level of the at least one gene product associated with a corresponding ADC therapy is weighted by a factor of approximately 0.45, the expression level of at least one gene product associated with cell adhesion is weighted by a factor of approximately −1, the determined proliferation gene expression level is weighted by a factor of approximately 0.55, and the tumor cellularity is weighted by a factor of approximately 0.07.

In some embodiments, each of the one or more ADC TRS is determined by further taking into account a bias variable, which is a static offset tuned to yield biomarker frequencies that match published objective response rates in clinical trials. In some embodiments, the bias variable is weighted by a factor of approximately −0.25.

In some embodiments, the predetermined threshold is set to a percentile of ranked ADC Treatment Response Scores determined from tumor tissue samples of a first cohort of subjects utilizing at least the expression level of the at least one gene product associated with a first corresponding ADC therapy, wherein each percentile corresponds to a percentage greater than a percentage of subjects of the first cohort which do not respond to the same first corresponding ADC therapy.

In some embodiments, the predetermined thresholds are set to percentiles of ranked ADC Treatment Response Scores determined from tumor tissue samples of a first and a second cohort of subjects utilizing at least the expression level of at least a first and a second gene product associated with a first and second corresponding ADC therapy, wherein each percentile corresponds to a percentage greater than a percentage of subjects of the first and second cohort which do not respond to the at least first and second corresponding ADC therapies.

In some embodiments, the predetermined threshold is set at zero, and an ADC TRS which indicates the subject is likely to benefit from an ADC therapy is an ADC TRS with a value greater than zero.

In some embodiments, the first and/or second cohort of subjects is a pan-cancer cohort or a matched tumor-type cohort. In some embodiments, the first cohort of subjects and the second cohort of subjects are the same cohort of subjects.

In some embodiments, the gene expression level of the at least one gene product associated with each one or more ADC therapies, the measured expression level of the at least one gene product associated with cell adhesion, and the tumor cellularity are log 2 transformed and/or Z score normalized prior to step (E)(1), and the expression level of the one or more gene products associated with proliferation are log 2 transformed and/or Z score normalized prior to the step of averaging expression levels of gene products associated with proliferation to obtain the proliferation gene expression level in step (C).

In some embodiments, the tumor cellularity is a molecularly determined tumor cellularity which is calculated based on somatic and germline variant allele frequencies and/or copy number profiles.

In some embodiments, the expression product of the at least one gene associated with each of the one or more ADC therapies, at least one gene associated with cell adhesion, and one or more genes associated with proliferation are individually selected from a ribonucleic acid (RNA) and a protein.

In some embodiments, the gene expression product of the at least one gene associated with each of the one or more ADC therapies, the at least one gene associated with cell adhesion, and the one or more genes associated with proliferation are proteins, and measuring the expression level thereof requires making use of immunohistochemistry techniques.

In some embodiments, the gene expression product of the at least one genes associated with each of the one or more ADC therapies, the at least one gene associated with cell adhesion, and the one or more genes associated with proliferation are RNA, and measuring the expression level thereof requires making use of RNA sequencing techniques.

In some embodiments, each of the one or more ADC therapies comprise a monoclonal antibody, at least one functional fragment thereof or a bispecific antibody which targets at least one epitope of at least one antigen selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5.

In some embodiments, the antibody comprises a bispecific antibody capable of targeting two epitopes of the same antigen or an epitope of two distinct antigens, wherein the same antigen or the two antigens are selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5.

In some embodiments, the antibody or fragment thereof is conjugated directly or indirectly to a cytotoxic drug. In some embodiments, the cytotoxic drug is a DNA replication inhibitor selected from the group consisting of an alkylating agent, a DNA polymerase inhibitor, a nitrogen mustard and a topoisomerase inhibitor. In some embodiments, the antibody, the at least one functional fragment thereof, or the bispecific antibody is fused to a protein which is toxic to a cancer cell. In some embodiments, cytotoxic drug is a topoisomerase inhibitor.

In some embodiments, the subject has or is suspected of having a cancer not approved for labeled use of the one or more ADC therapies. In some embodiments, the tumor tissue sample is or is suspected of containing bladder cancer, salivary gland cancer, endometrial cancer, ovarian cancer, cervical cancer, head and neck cancer, non-melanoma skin cancer, thyroid cancer, cancer of unknown primary, cancer of the central or peripheral nervous system, neuroendocrine tumor, melanoma, esophagogastric cancer, small bowel cancer, sarcoma, hepatobiliary cancer, pancreatic cancer, gastrointestinal stromal tumor, renal cell carcinoma, glioma, appendiceal cancer breast cancer, small cell lung cancer, prostate cancer, esophagogastric cancer, non-small cell lung cancer, pancreatic cancer, lymphoma or colorectal cancer.

In some embodiments, the tumor tissue sample is a formalin-fixed paraffin-embedded (FFPE) tumor tissue sample. In some embodiments, the tumor tissue sample contains at least 20% tumor content.

In some embodiments, the one or more ADC therapies comprise at least two therapies, and step (E)(1) comprises calculating at least two ADC Treatment Response Scores, wherein if the subject is identified likely to respond to at least two ADC therapies, the method further comprises step (E)(2) of ranking the at least two ADC Treatment Response Scores by the value by which each ADC Treatment Response Score exceeds the predetermined threshold, and identifying the highest ranked ADC therapy as the most likely to benefit the subject.

In another aspect, the invention is directed to a computer-implemented method for selecting a patient presenting with a solid cancerous tumor for treatment by one or more antibody-drug conjugate (ADC) therapies, wherein the method comprises the steps of: (A) receiving a measured expression level of at least one gene product associated with each of the one or more ADC therapies from a biological tissue sample obtained from the tumor tissue of the patient; (B)(1) receiving an expression level of one or both of: (i) at least one gene product associated with cell adhesion, and (ii) one or more gene products associated with proliferation, wherein the expression levels of (i) and (ii) are measured in the same biological tumor tissue sample of step (A), (B)(2) if the measured expression level of the one or more gene products associated with proliferation is received, calculating therefrom, with a computer, an average of all expression levels of the received measured gene products associated with proliferation, thereby obtaining a proliferation gene expression level; (C) optionally, receiving an indication of tumor cellularity of the same tumor tissue sample of steps (A) and (B); (D)(1) identifying a subject likely to respond to the one or more ADC therapies when either: (i) one or more of a calculated ADC Treatment Response Score (ADC TRS) surpass one or more corresponding predetermined thresholds, wherein each of the one or more ADC TRS is determined from: (1) the received measured expression level of the at least one gene product associated with a corresponding ADC therapy, and at least two of: (2) the received measured expression level of the at least one gene product associated with cell adhesion, (3) the calculated proliferation gene expression level from the received expression levels of one or more gene products associated with proliferation, and (4) the received indication of tumor cellularity; and (E) selecting the patient identified as likely to respond to the one or more ADC therapies for treatment therewith, wherein at least steps (A)-(D)(1)(i) are performed with a suitably programmed computer.

In some embodiments, step (E) comprises selecting the patient to receive treatment with the one or more ADC therapies as part of a clinical trial. In some embodiments, the clinical trial is a basket trial. In some embodiments, the method further comprises step (F) treating the selected patient with the one or more ADC therapies determined to likely induce a response in the patient.

In some embodiments, the ADC TRS is determined by at least the combination of the expression level of the at least one gene product associated with a corresponding ADC therapy, the expression level of the at least one gene product associated with cell adhesion, and the determined proliferation gene expression level. In some embodiments, both the determined proliferation gene expression level and the expression level of the at least one gene product associated with a corresponding ADC therapy are positively associated with a likelihood that a patient will benefit from the corresponding ADC therapy, and the expression level of the at least one gene product associated with cell adhesion is negatively associated with a likelihood that a patient will benefit from the same corresponding ADC therapy.

In some embodiments, the ADC TRS is determined by at least the combination of the expression level of the at least one gene product associated with a corresponding ADC therapy, the expression level of the at least one gene product associated with cell adhesion, and tumor cellularity. In some embodiments, both the tumor cellularity and the expression level of the at least one gene product associated with a corresponding ADC therapy are positively associated with a likelihood that a patient will benefit from the corresponding ADC therapy, and the expression level of the at least one gene product associated with cell adhesion is negatively associated with a likelihood that a patient will benefit from the same corresponding ADC therapy.

In some embodiments, the ADC TRS is determined by at least the combination of the expression level of the at least one gene product associated with a corresponding ADC therapy, the expression level of the at least one gene product associated with cell adhesion, the determined proliferation gene expression level, and tumor cellularity. In some embodiments, each of the determined proliferation gene expression level, the tumor cellularity, and the expression level of the at least one gene product associated with a corresponding ADC therapy are positively associated with a likelihood that a patient will benefit from the corresponding ADC therapy, and the expression level of the at least one gene product associated with cell adhesion is negatively associated with a likelihood that a patient will benefit from the same corresponding ADC therapy.

In some embodiments, each of the one or more ADC TRS is determined by further taking into account a bias variable, wherein the bias variable is a static offset tuned to yield biomarker frequencies that match published objective response rates in clinical trials.

In some embodiments, the predetermined threshold is set to a percentile of ranked ADC Treatment Response Scores determined from tumor tissue samples of a first cohort of subjects utilizing at least the expression level of the at least one gene product associated with a first corresponding ADC therapy, wherein each percentile corresponds to a percentage greater than a percentage of subjects of the first cohort which do not respond to the same first corresponding ADC therapy. In some embodiments, the first cohort of subjects is a pan-cancer cohort or a matched tumor-type cohort.

In some embodiments, the predetermined threshold is set at zero, and an ADC TRS which indicates the subject is likely to benefit from an ADC therapy is an ADC TRS with a value greater than zero.

In some embodiments, gene expression level of the at least one gene product associated with each one or more ADC therapies, the expression level of the at least one gene product associated with cell adhesion, and optionally the tumor cellularity are log 2 transformed and/or Z score normalized prior to step (D)(1), and the expression level of the one or more gene products associated with proliferation are log 2 transformed and/or Z score normalized prior to the step of averaging expression levels of gene products associated with proliferation to obtain the proliferation gene expression level in step (B)(2).

In some embodiments, the tumor cellularity is a molecularly determined tumor cellularity which is calculated based on somatic and germline variant allele frequencies and/or copy number profiles.

In some embodiments, the expression product of the at least one gene associated with each of the one or more ADC therapies, the at least one gene associated with cell adhesion, and one or more genes associated with proliferation are individually selected from a ribonucleic acid (RNA) and a protein.

In some embodiments, the gene expression product of the at least one gene associated with each of the one or more ADC therapies, the at least one gene associated with cell adhesion, and the one or more genes associated with proliferation are proteins, and measuring the expression level thereof requires making use of immunohistochemistry techniques.

In some embodiments, the gene expression product of the at least one gene associated with each of the one or more ADC therapies, the at least one gene associated with cell adhesion, and the one or more genes associated with proliferation are RNA, and measuring the expression level thereof requires making use of RNA sequencing techniques.

In some embodiments, each of the one or more ADC therapies comprise a monoclonal antibody, at least one functional fragment thereof or a bispecific antibody which targets at least one epitope of at least one antigen selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5. In some embodiments, the antibody or fragment thereof is conjugated directly or indirectly to a cytotoxic drug or fused to a cytotoxic protein.

In some embodiments, the subject has or is suspected of having a cancer not approved for labeled use of the one or more ADC therapies. In some embodiments, the tumor tissue sample is or is suspected of containing bladder cancer, salivary gland cancer, endometrial cancer, ovarian cancer, cervical cancer, head and neck cancer, non-melanoma skin cancer, thyroid cancer, cancer of unknown primary, cancer of the central or peripheral nervous system, neuroendocrine tumor, melanoma, esophagogastric cancer, small bowel cancer, sarcoma, hepatobiliary cancer, pancreatic cancer, gastrointestinal stromal tumor, renal cell carcinoma, glioma, appendiceal cancer breast cancer, small cell lung cancer, prostate cancer, esophagogastric cancer, non-small cell lung cancer, pancreatic cancer, lymphoma or colorectal cancer.

In some embodiments, the tumor tissue sample is a formalin-fixed paraffin-embedded (FFPE) tumor tissue sample. In some embodiments, the tumor tissue sample contains at least 20% tumor content.

In another aspect, the invention is directed to a method of identifying a subject as likely to benefit from anti-TROP2 based therapy, comprising: (a) measuring the expression level of a TROP2 gene product and one or more gene products associated with proliferation in a tumor tissue sample obtained from the subject; (b) measuring the expression levels of one or more housekeeping genes in the same tumor tissue sample of step (a), wherein the one or more housekeeping genes comprise three genes selected from CIAO1, EIF2B1, HMBS, CTCF, GGNBP2, ITGB7, MYC and SLC4A1AP, and further normalizing the expression levels of the TROP2 gene product and one or more gene products associated with proliferation of step (a) to the three housekeeping genes to obtain normalized expression levels of the TROP2 and one or more genes associated with proliferation; (c) determining proliferation gene expression level by averaging the normalized expression levels of the one or more gene products associated with proliferation; (d) determining tumor cellularity in the same tumor tissue sample of step (a); and (e) identifying the subject as likely to benefit from the anti-TROP2 based therapy when either i) an aggregate biomarker score surpasses a predetermined threshold, wherein the aggregate biomarker score is calculated from the combination of the measured expression level of the TROP2 gene product and at least one of the determined proliferation gene expression level and/or the determined tumor cellularity, or when ii) the measured expression level of the TROP2 gene product is higher than a median TROP2 expression level obtained from tumor tissue samples from a first cohort of subjects, and at least one of the determined proliferation gene expression level and/or the determined tumor cellularity is higher than a median proliferation gene expression level and/or a median tumor cellularity obtained from tumor tissue samples from the same first cohort of subjects.

In some embodiments, wherein the subject is identified as likely to benefit from the anti-TROP2 based therapy, when the measured expression level of the TROP2 gene product, the determined proliferation gene expression level, and the determined tumor cellularity are all higher than corresponding median levels of TROP2 expression, proliferation gene expression and median tumor cellularity obtained from tumor tissue samples from the same first cohort of subjects.

In some embodiments, the subject is identified as likely to benefit from the anti-TROP2 based therapy when one or more of the measured expression levels of the TROP2 gene product, the determined proliferation gene expression level, and/or the determined tumor cellularity fall into the highest quartile of TROP2 expression level, proliferation gene expression level and/or tumor cellularity values obtained from tumor tissue samples from the same first cohort of subjects.

In some embodiments, the first cohort of subjects is a pan-cancer cohort or a matched tumor-type cohort.

In some embodiments, measuring the expression level of one or more gene products associated with proliferation comprises measuring the expression levels of gene products of one or more genes selected from the group consisting of BIRC5, BRCA1, BRCA2, BUB1, BUB1B, CCNA2, CCNB1, CCNB2, CCNE1, CCNE2, CCNF, CCNG2, CDC2, CDC20, CDC25A, CDC25B, CDC25C, CDC45L, CDC6, CDC7, CDCKN1A p21, CDCKN3, CDK4, CDKN1C, CDKN2A, CDKN2C, CDKN2D p19, CDKN3, CENPA, CENPE, CENPF, CHAF1A, CHK1, CKS1, CKS2, CKS2, DHFR, DHFR, E2-EPF, E2F1, E2F3, E2F5, FEN1, FOXM1, KNSL2, KNSL5, KPNA2, LMNB2, MAD2, MAD2L1, MAPK13, MCM2, MCM3, MCM4, MCM5, MCM6, MK167, MNAT1, MSH2, MYBL2, NASP, NEK2, NPAT, ORC1, PA2G4, PCNA, PES1, PKMYT1, PLK, PRIM1, PTTG1, RAB6KIFL, RACGAP1, RAD51, RAD54L, RAN RAN, RFC4, RPA3, RRM1, RRM2, SLBP, STK12, STK15, STK18, STK6, TOP2A, TROAP, TUBB, TYMS, UBE2C, and UNG. In some embodiments, measuring the expression level of one or more gene products associated with proliferation comprises measuring the expression level of one, two or all three of MYBL2, TOP2A, and/or UBE2C gene products.

In some embodiments, the aggregate biomarker score is determined by the combination of the expression level of TROP2 gene products, the determined proliferation gene expression level, and tumor cellularity.

In some embodiments, the predetermined threshold is set to the percentile of ranked aggregate biomarker scores determined from tumor tissue samples of a second cohort of subjects, which percentile corresponds to a percentage of subjects of the second cohort which do not respond to an anti-TROP2 based therapy.

In some embodiments, the second cohort of subjects is a pan-cancer cohort or a matched tumor-type cohort. In some embodiments, the first cohort of subjects and the second cohort of subjects are the same cohort of subjects.

In some embodiments, gene expression levels of TROP2, one or more genes associated with proliferation, and tumor cellularity are log 2 transformed and/or median-centered to 10 prior to step (d).

In some embodiments, the tumor cellularity is a molecularly determined tumor cellularity which is calculated based on somatic and germline variant allele frequencies and/or copy number profiles.

In some embodiments, one, two or three of the housekeeping genes are selected from CIAO1, EIF2B1, and HMBS.

In some embodiments, the aggregate biomarker score is determined by adding the measured expression level of TROP2 gene product to approximately ⅓ to ⅔ the determined proliferation gene expression level and approximately 4-8 times the determined tumor cellularity.

In some embodiments, the expression products of TROP2 and one or more genes associated with proliferation are individually selected from a ribonucleic acid (RNA) and a protein.

In some embodiments, the gene expression product of at least one of TROP2 and one or more genes associated with proliferation is a protein, and measuring the expression level thereof requires making use of immunohistochemistry techniques.

In some embodiments, the gene expression product of at least one of TROP2 and one or more genes associated with proliferation is a RNA, and measuring the expression level thereof requires making use of RNA sequencing techniques.

In some embodiments, the anti-TROP2 based therapy, comprises an anti-TROP2 antibody or fragment thereof.

In some embodiments, the anti-TROP2 antibody or fragment thereof is conjugated directly or indirectly to a cytotoxic drug. In some embodiments, the cytotoxic drug is a DNA replication inhibitor selected from the group consisting of an alkylating agent, a DNA polymerase inhibitor, a nitrogen mustard and a topoisomerase inhibitor.

In some embodiments, the anti-TROP2 antibody or fragment thereof is fused to a protein which is toxic to a cancer cell. In some embodiments, the cytotoxic drug is a topoisomerase inhibitor. In some embodiments, the anti-TROP2 based therapy is Sacituzumab govitecan.

In some embodiments, the subject has or is suspected of having a cancer not approved for labeled use of anti-TROP2 based therapy. In some embodiments, the tumor tissue sample is or is suspected of containing bladder cancer, endometrial cancer, breast cancer, small cell lung cancer, prostate cancer, esophagogastric cancer, non-small cell lung cancer, pancreatic cancer, or colorectal cancer.

In some embodiments, the tumor tissue sample is a formalin-fixed paraffin-embedded (FFPE) tumor tissue sample. In some embodiments, the tumor tissue sample contains at least 20% tumor content.

In some embodiments, the method further comprises administering an anti-TROP2 based therapy to the subject identified as likely to benefit from the anti-TROP2 based therapy.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 depicts correlation analysis of the SG biomarker positive rate in the validation cohort with objective response rate observed in the IMMU-12-01 basket trials.

FIG. 2 depicts biomarker distributions across the full molecular cohort for TROP2 expression.

FIG. 3 depicts biomarker distributions across the full molecular cohort for proliferation gene expression.

FIG. 4 depicts biomarker distributions across the full molecular cohort for tumor cellularity.

FIG. 5 depicts comparison of TROP2 gene expression by RNA sequencing (% above pan-solid tumor median) to TROP2 protein expression by immunohistochemistryo (% with moderate or strong staining) across 45 tumor types.

FIG. 6 depicts biomarker relationships in the full molecular cohort for (A) TOP2A vs. UBE2C expression, (B) TROP2 expression vs. proliferation gene expression, (C) TROP2 expression vs. tumor cellularity, (D) proliferation gene expression vs. tumor cellularity.

FIG. 7 depicts individual biomarker rate correlations with objective response rate for (A) TROP2, (B) proliferation gene expression and (C) tumor cellularity.

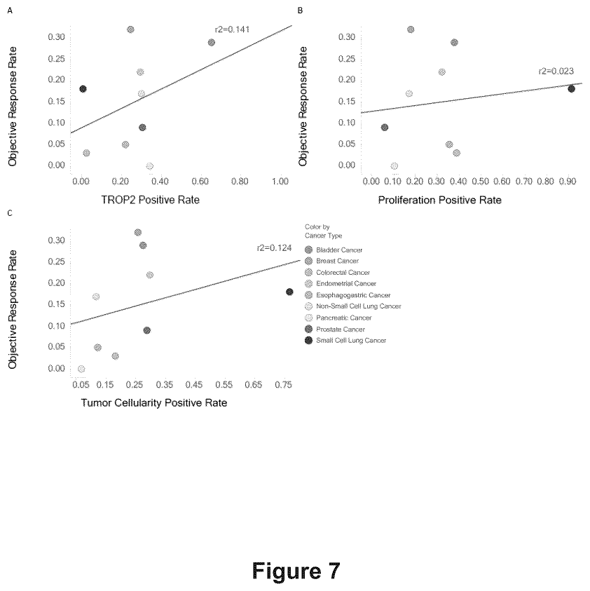

FIG. 8 depicts correlation analysis of the SG biomarker positive rate in the discovery cohort with objective response rate observed in the IMMU-12-01 basket trials.

FIG. 9 depicts SG biomarker status as related to the biomarker factors in the full molecular cohort: TROP2 expression (y-axis), proliferation gene expression (x-axis) and tumor cellularity (binned by panel). Biomarker positive samples are colored red and biomarker negative samples are colored blue.

FIG. 10 depicts correlation analysis of the observed objective response rates across 22 clinical trials and cohorts across 9 antibody-drug conjugate therapies.

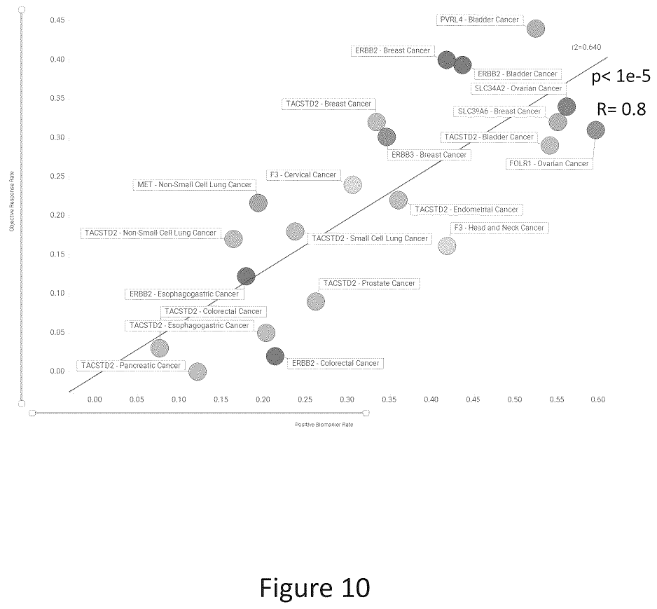

FIG. 11 depicts a heatmap showing biomarker positive rates across 10 antibody-drug conjugates and 28 tumor types.

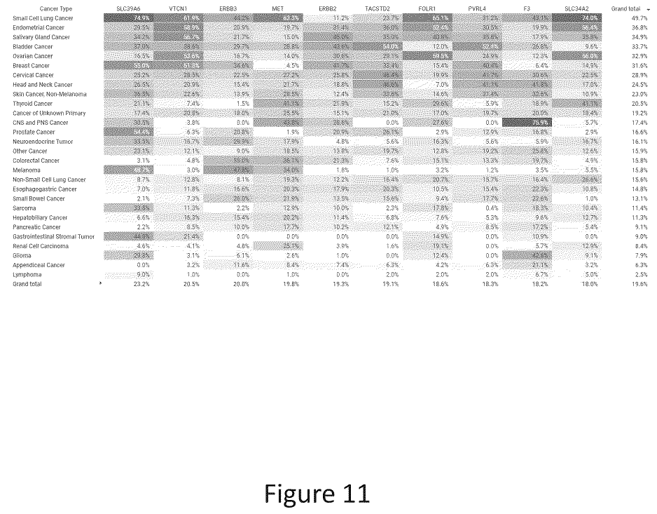

FIG. 12 depicts the best antibody-drug conjugate response scores per patient and summarized by tumor type.

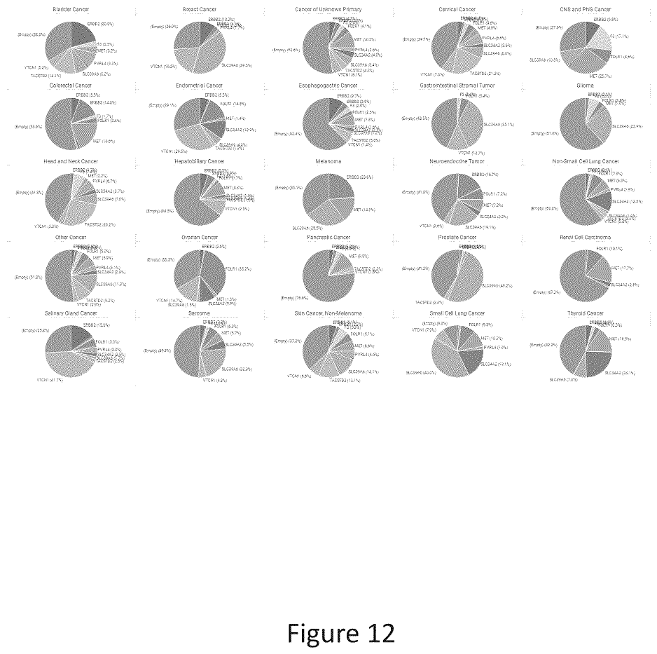

FIG. 13 depicts the best antibody-drug conjugate response scores overall.

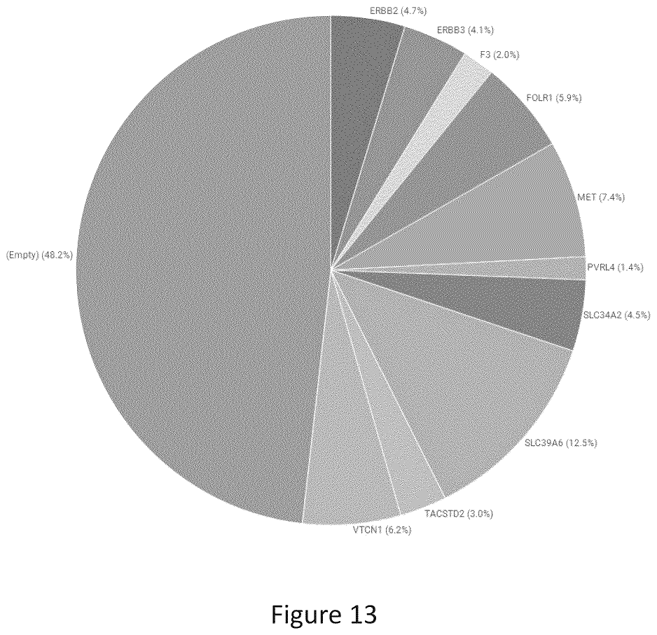

FIG. 14 lists the most common antibody-drug conjugates response scores biomarker profiles, ranked by number of called target counts.

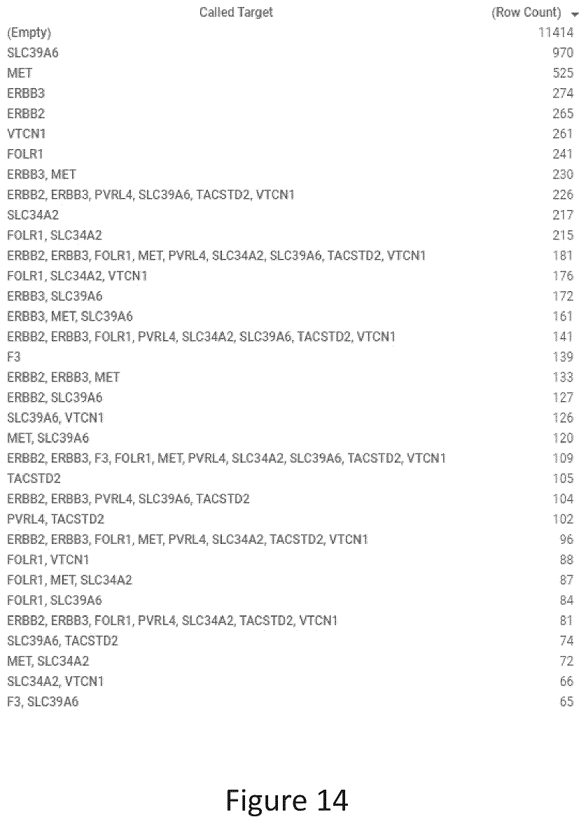

FIG. 15 lists absolute correlation between top 20 gene expression level or Copy Number Genes and ADC ORRs.

FIG. 16 depicts a model architecture for the ADC Treatment Response Score that comprises ADC target expression level, proliferation gene expression level, PVR gene expression, as well as a bias input, with a positive biomarker call set to a threshold of >0.

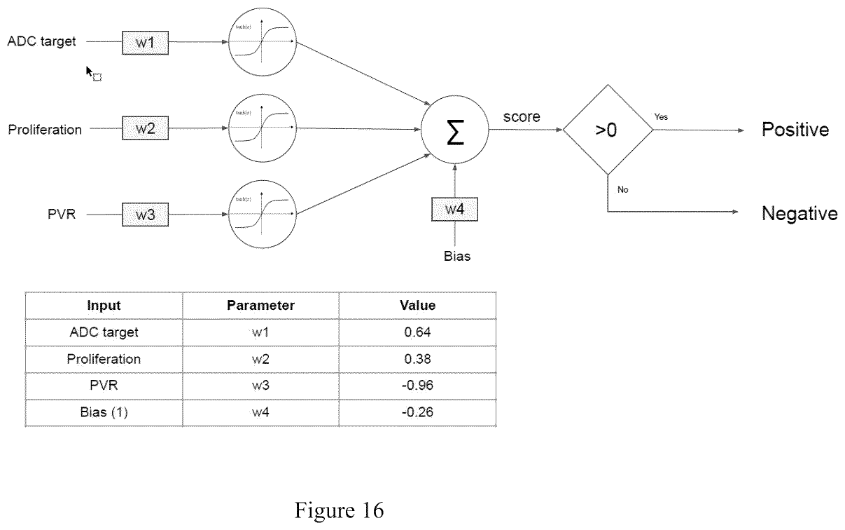

FIG. 17 depicts biomarker rate correlations with objective response rate for a biomarker comprising ADC target gene product expression, proliferation gene expression and PVR gene expression.

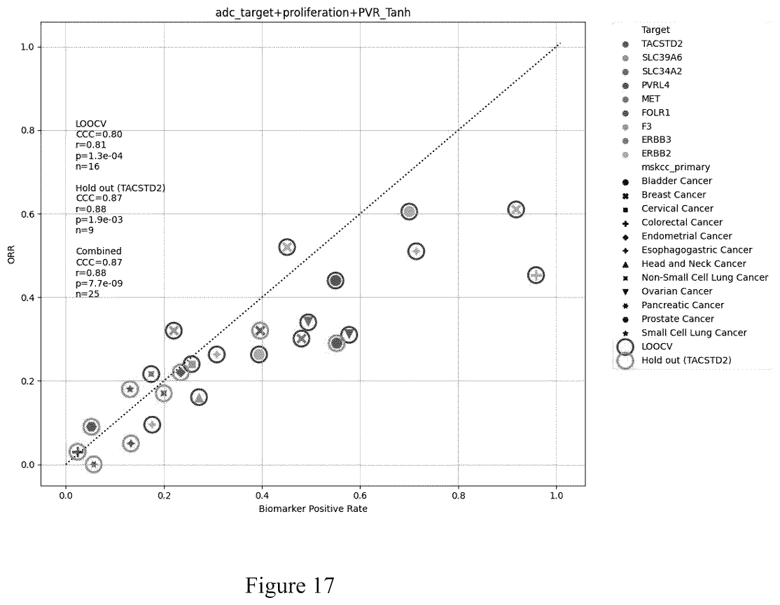

FIG. 18 depicts a heatmap representing predicted biomarker positivity rates for different cancers and with 9 different ADCs. The outlined boxes indicate availability of a corresponding published ORR.

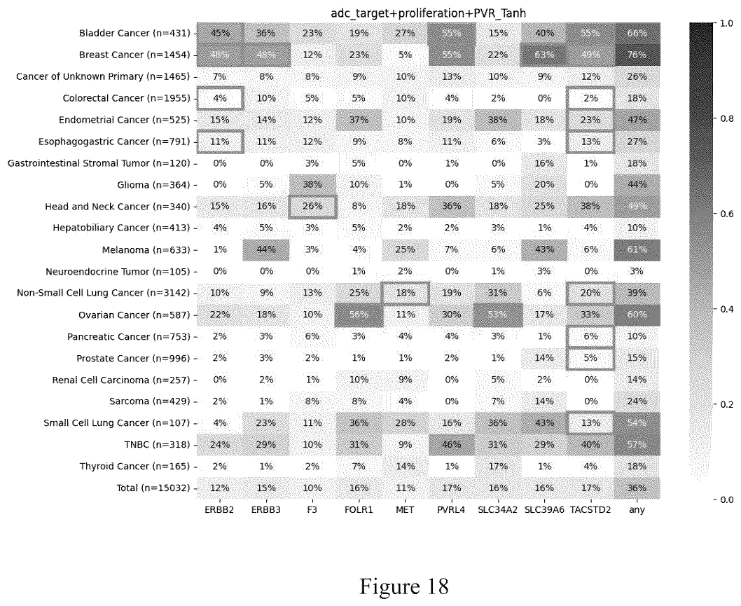

FIG. 19 depicts biomarker rate correlations with objective response rates for a biomarker comprising ADC target gene product expression, provided for comparative purposes. As can be seen by the low concordance correlation coefficient of 0.46, ADC target expression is a poor predictor of objective response rates to the ADC therapy.

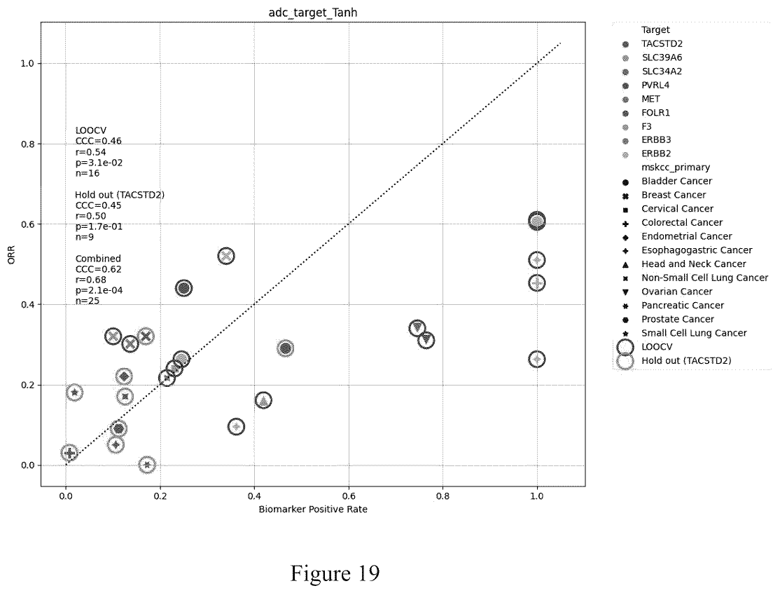

FIG. 20 depicts biomarker rate correlations with objective response rates for a biomarker comprising ADC target gene product expression and tumor cellularity (tumor content), provided for comparative purposes.

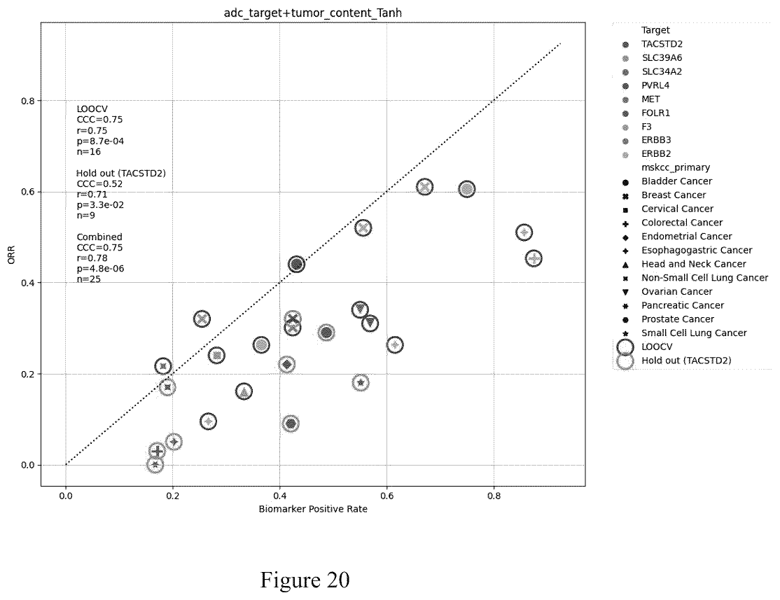

FIG. 21 depicts biomarker rate correlations with objective response rates for a biomarker comprising ADC target gene product expression and proliferation gene expression, provided for comparative purposes.

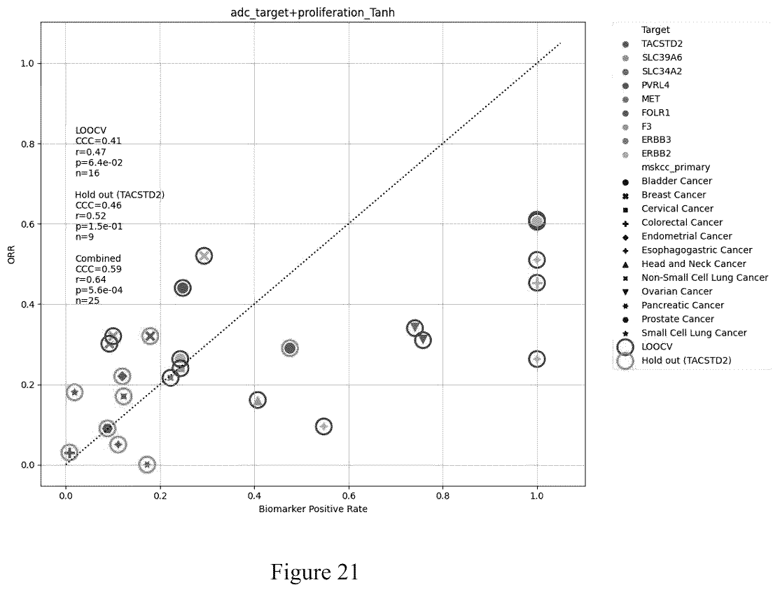

### DETAILED DESCRIPTION OF THE INVENTION

### Some Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “antibody” herein is used in the broadest sense and encompasses various antibody structures, including but not limited to monoclonal antibodies, polyclonal antibodies, and multispecific antibodies (e.g., bispecific antibodies).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “human antibody” is an antibody that possesses an amino acid sequence corresponding to that of an antibody produced by a human and/or has been made using any of the techniques for making human antibodies known to one of skill in the art. This definition of a human antibody specifically excludes a humanized antibody comprising non-human antigen-binding residues. Human antibodies can be produced using various techniques known in the art, including methods described in Cole et al, Monoclonal Antibodies and Cancer Therapy, Alan R. Liss, p. 77 (1985); Boerner et al, J. Immunol, 147(I):86-95 (1991). See also van Dijk and van de Winkel, Curr. Opin. Pharmacol, 5: 368-74 (2001). Human antibodies can be prepared by administering the antigen to a transgenic animal that has been modified to produce such antibodies in response to antigenic challenge, but whose endogenous loci have been disabled, e.g., immunized HuMab mice (see, e.g., Nils Lonberg et al., 1994, Nature 368:856-859, WO 98/24884, WO 94/25585, WO 93/1227, WO 92/22645, WO 92/03918 and WO 01/09187 regarding HuMab mice), xenomice (see, e.g., U.S. Pat. Nos. 6,075,181 and 6,150,584 regarding XENOMOUSE™ technology) or Trianni mice (see, e.g., WO 2013/063391, WO 2017/035252 and WO 2017/136734 regarding Trianni mice).

The term “humanized antibody” refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those skilled in the art will be aware of humanized antibodies and will also be aware of suitable techniques for their generation. See for example, Hwang, W. Y. K., et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and Selick et al., WO 90/07861, each of which is incorporated herein by reference in its entirety.

As used herein, the term “bispecific antibodies” refers to monoclonal, often human or humanized, antibodies that have binding specificities for at least two different antigens. In the invention, one of the binding specificities can be directed towards CLDN18.2, the other can be for any other antigen, e.g., for a cell-surface protein, receptor, receptor subunit, tissue-specific antigen, virally derived protein, virally encoded envelope protein, bacterially derived protein, or bacterial surface protein, etc.

The term “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab)₂; diabodies; linear antibodies; single-chain antibody molecules (e.g., scFv). Papain digestion of antibodies produces two identical antigen-binding fragments, called “Fab” fragments, and a residual “Fc” fragment, a designation reflecting the ability to crystallize readily. The Fab fragment consists of an entire light (L) chain (VL) along with the variable region domain of the heavy (H) chain (VH), and the first constant domain of one heavy chain (CH1). Pepsin treatment of an antibody yields a single large F(ab)₂ fragment which roughly corresponds to two disulfide linked Fab fragments having divalent antigen-binding activity and is still capable of cross-linking antigen. Fab fragments differ from F(ab)₂ fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “expression” refers to the cellular processes involved in producing RNA and proteins and as appropriate, secreting proteins, including where applicable, but not limited to, transcription, translation, folding, modification and processing. “Expression products” include RNA transcribed from a gene and polypeptides obtained by translation of mRNA transcribed from a gene.

The term “RNA” is defined as ribonucleic acid.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The terms “non-human animals” and “non-human mammals” as used herein interchangeably, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treating” and “treatment” refer to administering to a subject an effective amount of a composition so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

The terms “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

Methods of Identifying a Subject Likely to Benefit from an ADC Therapy and Methods of Selecting a Subject for Treatment with an ADC Therapy

In certain aspects, the invention is directed to a method of identifying a cancer subject as likely to respond to one or more antibody-drug conjugate (ADC) therapies based upon a gene product expression profile measured in a tissue sample obtained from the subject. In some embodiments, the method comprises (A) measuring the expression level of at least one gene product associated with each of the one or more ADC therapies from a biological tissue sample obtained from the subject, (B) measuring in the same biological tissue sample of step (A), the expression level of one or both of i) at least one gene product associated with cell adhesion, and ii) one or more gene products associated with proliferation, wherein if the expression level of more than one gene product associated with proliferation is measured, calculating therefrom an average of all expression levels of the measured gene products associated with proliferation, thereby obtaining a proliferation gene expression level, and (D)(1) identifying a cancer subject as likely to respond to the one or more ADC therapies when (i) the measured expression level of the at least one gene product associated with a corresponding ADC therapy, and at least one of (ii) the measured expression level of at least one gene product associated with cell adhesion, and (iii) the measured expression level of one or more gene products associated with proliferation exceed predetermined thresholds, or alternatively, when an ADC Treatment Response Score calculated therefrom surpass a corresponding predetermined threshold associated with a positive response to the one or more ADC therapies.

In some embodiments, the step of measuring at least one gene product associated with each of the one or more ADC therapies comprise quantifying gene products of one or more of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5. In some embodiments, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, or thirty-one distinct gene products are measured. In some embodiments, at least two, at least five, at least ten, at least fifteen, or at least twenty gene products are measured.

In some embodiments, the step of measuring the expression level of one or more gene products associated with proliferation comprises measuring the expression levels of a gene product of one or more genes which are cell cycle regulated and pertain to DNA replication, mitotic processes/phases, spindle assembly, tubulin, mitotic surveillance, cell adhesion, chromosome metabolism, and histone formation. In some embodiments genes with gene products associated with proliferation are one or more genes selected from the group consisting of BIRC5, BRCA1, BRCA2, BUB1, BUB1B, CCNA2, CCNB1, CCNB2, CCNE1, CCNE2, CCNF, CCNG2, CDC2, CDC20, CDC25A, CDC25B, CDC25C, CDC45L, CDC6, CDC7, CDCKN1A p21, CDCKN3, CDK4, CDKN1C, CDKN2A, CDKN2C, CDKN2D p19, CDKN3, CENPA, CENPE, CENPF, CHAF1A, CHK1, CKS1, CKS2, CKS2, DHFR, DHFR, E2-EPF, E2F1, E2F3, E2F5, FEN1, FOXM1, KNSL2, KNSL5, KPNA2, LMNB2, MAD2, MAD2L1, MAPK13, MCM2, MCM3, MCM4, MCM5, MCM6, MK167, MNAT1, MSH2, MYBL2, NASP, NEK2, NPAT, ORC1, PA2G4, PCNA, PES1, PKMYT1, PLK, PRIM1, PTTG1, RAB6KIFL, RACGAP1, RAD51, RAD54L, RAN RAN, RFC4, RPA3, RRM1, RRM2, SLBP, STK12, STK15, STK18, STK6, TOP2A, TROAP, TUBB, TYMS, UBE2C, and UNG. In some embodiments, measuring the expression level of one or more gene products associated with proliferation comprises measuring the expression level of one, two or all three of MYBL2, TOP2A, and/or UBE2C gene products.

The inventors have recently discovered that adding an additional variable enhanced predictive power of the method in predicting a given patient response to ADC therapy. Thus, in some embodiments, the ADC TRS is determined by at least the combination of an expression level of at least one gene product associated with a corresponding ADC therapy, one or both of tumor cellularity and proliferation gene expression level, as well as the expression level of one additional gene product. In some embodiments, the predictive power of the ADC TRS can be enhanced by the addition of a variable comprising an expression level of at least one gene product associated with cell adhesion. Consequently, in certain embodiments, the ADC TRS is determined by one gene product associated with a corresponding ADC therapy, a proliferation gene expression level, as well as the expression level of at least one gene product associated with cell adhesion.

In some embodiments, both the determined proliferation gene expression level and the expression level of the at least one gene product associated with a corresponding ADC therapy are positively associated with a likelihood that a patient will respond to the corresponding ADC therapy, and the expression level of the at least one gene product associated with cell adhesion is negatively associated with a likelihood that a patient will respond to the same corresponding ADC therapy. In some embodiments, the expression level of the at least one gene product associated with a corresponding ADC therapy is weighted by a factor of approximately 0.3 to 0.7, approximately 0.35 to 0.65, approximately 0.4 to approximately 0.5, or approximately 0.4, 0.45, 0.48, 0.50, 0.55, 0.60, 0.65, 0.68, or 0.7. In some embodiments, the expression level of the at least one gene product associated with cell adhesion is weighted by a factor of approximately −1.5 to −0.5, −1.25 to −0.75, −1.1 to −0.8, −1 to −0.8, or approximately −1.05, −1.0, −0.95, −0.90 or −0.85. In some embodiments, the determined proliferation gene expression level is weighted by a factor of approximately 0.15 to 0.60, approximately 0.20 to 0.40, or approximately 0.45, 0.40, 0.38, 0.35, 0.30, or 0.25.

According to one aspect of the invention, the gene product associated with cell adhesion is a gene product providing an expression level in a cohort of cancer patients which is negatively correlated with the objective response rate to at least one ADC therapy in this same cohort of cancer patients. The statistical methods used for determining the negative correlation between objective response rates and expression level of a gene product associated with cell adhesion is not particularly limited, and will be within the ordinary skill of one in this field, and includes those methods well known in the field, such as Spearman correlation or Pearson correlation. In some embodiments, the correlation coefficients associated with each cell adhesion associated gene product obtained by such analysis can be ranked and selected based upon its proximity to −1. Thus, in some embodiments, a cancer patient is deemed likely to respond to at least one ADC therapy only when the expression of at least one gene product associated with cell adhesion is reduced compared to a reference level. In some embodiments, the reference level is the mean expression level of the same gene product associated with cell adhesion in cancer subjects of an ADC therapy treatment cohort. In some embodiments, a cancer patient is deemed likely to respond to at least one ADC therapy only when the expression level of at least one gene product associated with cell adhesion falls at least two standard deviations below a mean expression level of the same gene product in cancer subjects of an ADC therapy treatment cohort.

In some embodiments, the gene product associated with cell adhesion is a gene product which impacts a cellular component of cell adhesion comprising an adherens junction, an anchoring junction, a cell-substrate adherens junction, a cell-substrate junction, and a focal adhesion. In certain embodiments the gene product associated with cell adhesion impacts at least two cellular components selected from the group consisting of an adherens junction, an anchoring junction, a cell-substrate adherens junction, a cell-substrate junction, and a focal adhesion. In some embodiments, the gene product associated with cell adhesion impacts at least three, at least four or all five of the cellular components. In some embodiments, the gene product associated with cell adhesion is chosen both because of its negative correlation to objective response rates and because it impacts all five cellular components selected from the group consisting of an adherens junction, an anchoring junction, a cell-substrate adherens junction, a cell-substrate junction, and a focal adhesion. By way of non-limiting example, Table 3 outlines genes which provide expression levels of gene products associated with cell adhesion which are negatively correlated with an objective response rate in cancer subjects of a cohort and the specific cellular components of cell adhesion impacted by their respective expression levels as discussed above.

In some embodiments, the step of measuring the expression level of at least one gene product associated with cell adhesion comprises measuring the expression level of a gene product of at least one gene selected from the group consisting of ATP2A2, BAIAP2, CD151, CHP1, CYFIP1, CYTH3, DAG1, DSC2, GIT1, HSP90B1, HSPA5, LIMK1, MAPK1, PACSIN2, PDIA3, PVR, REXO2, RPL22, RPLP1, RPLP2, RPS11, RPS16, RPS5, SDCBP SNAP23, SNTB1, and SRP68. In certain embodiments, the expression levels of two or more gene products of two or more genes selected from the group consisting of ATP2A2, BAIAP2, CD151, CHP1, CYFIP1, CYTH3, DAG1, DSC2, GIT1, HSP90B1, HSPA5, LIMK1, MAPK1, PACSIN2, PDIA3, PVR, REXO2, RPL22, RPLP1, RPLP2, RPS11, RPS16, RPS5, SDCBP SNAP23, SNTB1, and SRP68 are measured. When expression levels of two or more of the gene products associated with cell adhesion are measured, an average expression level may be calculated to provide a cell adhesion gene product expression level. Accordingly, in some embodiments, a cancer patient is deemed likely to respond to an ADC therapy only when the cell adhesion gene product expression level is reduced compared to a reference level. Prior to averaging the multiple expression levels of gene products associated with cell adhesion, each gene product expression level may be normalized and/or log 2 transformed. To facilitate the normalization of each gene product expression level, a mean and standard deviation of the corresponding gene product expression levels in a cancer patient cohort can be used. Thus, in some embodiments, a patient is identified as likely to respond to an ADC therapy only when a normalized cell adhesion gene product expression level is less than zero.

In one embodiment, the step of measuring the expression level of at least one gene product associated with cell adhesion comprises measuring the expression of a PVR gene product. In addition, at least one, two, three, four, five, ten, fifteen, twenty, twenty-five, or thirty additional gene products associated with cell adhesion can be measured along with the PVR gene product. In some embodiments, the step of measuring the expression level of at least one gene product associated with cell adhesion consists of measuring the expression level of a single gene product associated with cell adhesion. In some embodiments, only the expression of a PVR gene product is measured. In some embodiments, only the expression of a SRP68 gene product is measured. In some embodiments, only the expression of a SNTB1 gene product is measured. In some embodiments, only the expression of a SNAP23 gene product is measured. In some embodiments, only the expression of a SDCBP gene product is measured. In some embodiments, only the expression of a RPS5 gene product is measured. In some embodiments, only the expression of a RPS16 gene product is measured. In yet another embodiment, only the expression of a RPS11 gene product is measured. In some embodiments, only the expression of a RPLP2 gene product is measured. In some embodiments, only the expression of a RPLP1 gene product is measured. In some embodiments, only the expression of a REXO2 gene product is measured. In some embodiments, only the expression of a PDIA3 gene product is measured. In some embodiments, only the expression of a PACSIN2 gene product is measured. In some embodiments, only the expression of a MAPK1 gene product is measured. In some embodiments, only the expression of a LIMK1 gene product is measured. In some embodiments, only the expression of a HSPA5 gene product is measured. In some embodiments, only the expression of a HSP90B1 gene product is measured. In some embodiments, only the expression of a GIT1 gene product is measured. In some embodiments, only the expression of a DSC2 gene product is measured. In some embodiments, only the expression of a DAG1 gene product is measured. In some embodiments, only the expression of a CYTH3 gene product is measured. In some embodiments, only the expression of a CYFIP1 gene product is measured. In some embodiments, only the expression of a CHP1 gene product is measured. In some embodiments, only the expression of a CD151 gene product is measured. In some embodiments, only the expression of a BAIAP2 gene product is measured. In some embodiments, only the expression of a ATP2A2 gene product is measured.

In some embodiments, the ADC TRS is determined by at least the combination of the expression level of the at least one gene product associated with a corresponding ADC therapy, the expression level of the at least one gene product associated with cell adhesion, the determined proliferation gene expression level, and tumor cellularity. In some embodiments, both the determined proliferation gene expression level, the tumor cellularity, and the expression level of the at least one gene product associated with a corresponding ADC therapy are positively associated with a likelihood that a patient will respond to the corresponding ADC therapy, and the expression level of the at least one gene product associated with cell adhesion is negatively associated with a likelihood that a patient will respond to the same corresponding ADC therapy.

In some embodiments, the expression level of the at least one gene product associated with a corresponding ADC therapy is weighted by a factor of approximately 0.30 to 0.70, approximately 0.35 to 0.65, approximately 0.4 to approximately 0.5, or approximately 0.40, 0.45, 0.48, or 0.50. In some embodiments, the expression level of the at least one gene product associated with cell adhesion is weighted by a factor of approximately −1.5 to −0.5, −1.25 to −0.75, −1.1 to 0.8, or approximately −1.05, −1.0, −0.95, −0.90 or −0.85. In some embodiments, the determined proliferation gene expression level is weighted by a factor of approximately 0.75 to 0.35, 0.65 to 0.40, 0.60 to 0.50, or approximately 0.65, 0.60, 0.55, 0.50, or 0.45. In some embodiments, the tumor cellularity is weighted by a factor of approximately 0.25 to 0.01, 0.20 to 0.02, 0.17 to 0.04, or approximately 0.09, 0.08, 0.07, 0.06, or 0.05. In some embodiments, the expression level of the at least one gene product associated with a corresponding ADC therapy is weighted by a factor of approximately 0.45, the expression level of the at least one gene product associated with cell adhesion is weighted by a factor of approximately −0.98, the determined proliferation gene expression level is weighted by a factor of approximately 0.55, and the tumor cellularity is weighted by a factor of approximately 0.07.

In some embodiments, each of the one or more ADC TRS is determined by further taking into account a bias variable, wherein the bias variable is a static offset tuned to yield biomarker frequencies that match published objective response rates in clinical trials. In some embodiments, the bias variable is weighted by a factor of approximately −0.65 to −0.1, −0.55 to −0.15, −0.35 to 0.20, or approximately −0.22, −0.23, −0.24, −0.25, −0.25, −0.26, −0.27, or −0.28. In some embodiments the bias variable is weighted by a factor of approximately −0.26.

As discussed above, the ADC TRS is determined by multiple variables that are weighted individually based upon their influence on the probability that a subject is likely to response to an ADC therapy. When these variables are combined, a score is obtained which indicates a subject is likely to respond to an ADC therapy when it surpasses a certain predetermined threshold. In some embodiments, the predetermined threshold is zero, and an ADC TRS which indicates the subject is likely to respond to an ADC therapy is an ADC TRS with a value greater than zero.

In some embodiments, the predetermined threshold is set to a percentile of ranked ADC Treatment Response Scores determined from tumor tissue samples of a first cohort of subjects utilizing at least the expression level of the at least one gene product associated with a first corresponding ADC therapy, wherein each percentile corresponds to a percentage greater than a percentage of subjects of the first cohort which do not respond to the same first corresponding ADC therapy.

In some embodiments, the predetermined thresholds are set to percentiles of ranked ADC Treatment Response Scores determined from tumor tissue samples of a first and a second cohort of subjects utilizing at least the expression level of at least a first and a second gene product associated with a first and second corresponding ADC therapy, wherein each percentile corresponds to a percentage greater than a percentage of subjects of the first and second cohort which do not respond to the at least first and second corresponding ADC therapies.

In some embodiments, the first cohort of subjects is a pan-cancer cohort or a matched tumor-type cohort. In some embodiments, the first cohort is a matched tumor-type cohort. The matched tumor-type cohort is not particularly limited and includes those tumors/cancer known to those skilled in the art and disclosed herein. In some embodiments, the matched tumor-type is appendiceal cancer, bladder cancer, breast cancer, cervical cancer, CNS and PNS cancer, colorectal cancer, endometrial cancer, esophagogastric cancer, gastrointestinal stromal tumor, glioma, head and neck cancer, hepatobiliary cancer, lymphoma, melanoma, neuroendocrine tumor, non-small cell lung cancer, ovarian cancer, prostate cancer, renal cell carcinoma, salivary gland cancer, sarcoma, non-melanoma skin cancer, small bowel cancer, small cell lung cancer, or thyroid cancer.

In still other embodiments, the first cohort is a pan-cancer cohort. Such pan-cancer cohorts may contain 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more different cancers represented in the cohort. In certain embodiments, said cohort contains three or more cancers represented from the group consisting of appendiceal cancer, bladder cancer, breast cancer, cervical cancer, CNS and PNS cancer, colorectal cancer, endometrial cancer, esophagogastric cancer, gastrointestinal stromal tumor, glioma, head and neck cancer, hepatobiliary cancer, lymphoma, melanoma, neuroendocrine tumor, non-small cell lung cancer, ovarian cancer, prostate cancer, renal cell carcinoma, salivary gland cancer, sarcoma, non-melanoma skin cancer, small bowel cancer, small cell lung cancer, and thyroid cancer.

In some embodiments the first cohort is a clinical trial cohort comprising cancer patients who are receiving at least one antibody-drug conjugate therapy. In some embodiments, the patients are selected for inclusion in the clinical trial cohort on account of having an advanced cancer and displaying expression of one target of a candidate antibody-drug conjugate therapy. In some embodiments the advanced cancer has been radiologically confirmed to have metastasized or has been determined to be relapsing or refractory to a prior treatment with a chemotherapeutic drug. In some embodiments, the patient has an advanced solid tumor. In a preferred embodiment the cohort comprises at least 50 patients, at least 75 patients, at least 100 patients, at least 125 patients, at least 150 patients, at least 175 patients, at least 200 patients, at least 250 patients, at least 300 patients, at least 400 patients, at least 500 patients or more. In some embodiments, the cohort has at least 200 patients. In some embodiments, the clinical trial cohort is a basket trial/pan-cancer cohort. In some embodiments, the first cohort is a tumor specific cohort.

In some embodiments, the ADC Treatment Response Score is determined by the combination of the expression level of at least one gene product associated with each of the one or more corresponding ADC therapies, a determined proliferation gene expression level, and tumor cellularity. In some embodiments, the predetermined threshold is set to the percentile of ranked ADC Treatment Response Scores determined from tumor tissue samples of a second cohort of subjects, which percentile corresponds to a percentage of subjects of the second cohort which do not respond to the one or more ADC therapies.

In some embodiments, the second cohort of subjects is a pan-cancer cohort or a matched tumor-type cohort. In some embodiments, the second cohort is a matched tumor-type cohort. The matched tumor-type cohort is not particularly limited and includes those tumors/cancer known to those skilled in the art and disclosed herein. In some embodiments, the matched tumor-type is appendiceal cancer, bladder cancer, breast cancer, cervical cancer, CNS and PNS cancer, colorectal cancer, endometrial cancer, esophagogastric cancer, gastrointestinal stromal tumor, glioma, head and neck cancer, hepatobiliary cancer, lymphoma, melanoma, neuroendocrine tumor, non-small cell lung cancer, ovarian cancer, prostate cancer, renal cell carcinoma, salivary gland cancer, sarcoma, non-melanoma skin cancer, small bowel cancer, small cell lung cancer, or thyroid cancer.

In still other embodiments, the second cohort is a pan-cancer cohort. Such pan-cancer cohorts may contain 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more different cancers represented in the cohort. In certain embodiments, said cohort contains three or more cancers selected from the group consisting of cancer appendiceal cancer, bladder cancer, breast cancer, cervical cancer, CNS and PNS cancer, colorectal cancer, endometrial cancer, esophagogastric cancer, gastrointestinal stromal tumor, glioma, head and neck cancer, hepatobiliary cancer, lymphoma, melanoma, neuroendocrine tumor, non-small cell lung cancer, ovarian cancer, prostate cancer, renal cell carcinoma, salivary gland cancer, sarcoma, non-melanoma skin cancer, small bowel cancer, small cell lung cancer, and thyroid cancer.

In some embodiments, the first cohort of subjects and the second cohort of subjects are the same cohort of subjects.

In some embodiments, gene expression level of the at least one gene product associated with each one or more ADC therapies, the expression level of the at least one gene product associated with cell adhesion, and the optional tumor cellularity are log 2 transformed and/or Z score normalized prior to step (D)(1), and the expression level of the one or more gene products associated with proliferation are log 2 transformed and/or Z score normalized prior to the step of averaging expression levels of gene products associated with proliferation to obtain the proliferation gene expression level in step (B).

As discussed above, it is preferable that the gene expression level of the one or more gene products associated with ADC therapies, proliferation, cell adhesion, and optionally the tumor cellularity values are subjected to normalization to minimize the impact of outliers and to facilitate enhanced predictive power of the method. In some embodiments, the normalization is Z-score normalization. In some embodiments the normalization is min-max normalization. In some embodiments, the normalization is Z-score normalization based upon data from a from a large number of solid tumors, such as data obtained from at least 5,000, at least 10,000, at least 15,000, or at least 20,000 distinct solid tumor samples. In some embodiments, Z-score normalization is based upon data from more than 15,000 solid tumors in the Strata Trial (NCT03061305).

The process for Z score normalization of the gene expression level of the at least one gene product associated with each one or more ADC therapies, the one or more gene products associated with proliferation, the at least one gene product associated with cell adhesion, and the optional tumor cellularity, will be well understood by one of skill in the art. Generally, Z-score normalization is performed by first determining a mean and standard deviation for a given dataset, and then subsequently normalizing a datapoint by subtracting the median therefrom and dividing by the calculated standard deviation. For example, a mean and standard deviation are determined for expression levels of a gene product associated with an ADC therapy based upon data from over 10,000 solid tumor samples. Subsequently, a patient's expression level for this same gene product would be subtracted from the calculated mean and divided by the calculated standard deviation to provide a Z-score normalized expression value. This would then be repeated for the one or more gene products associated with proliferation, the tumor cellularity, and any additional gene products associated with each one or more ADC therapies, to thus provide Z-score normalized data for use in the method.

In some embodiments, the method further comprises measuring the expression level of at least one housekeeping gene selected from CIAO1, EIF2B1, and HMBS in the tumor tissue sample, and normalizing the expression levels of the at least one gene product associated with the one or more ADC therapies, the at least one gene product associate with cell adhesion and the one or more gene products associated with proliferation to the at least one housekeeping gene expression to obtain normalized expression levels of the at least one gene product associated with each of the one or more ADC therapies, the at least one gene product associated with cell adhesion, and the one or more gene products associated with proliferation. Thus, in some embodiments, the method comprises (a) measuring, in a tumor tissue sample obtained from the subject, the expression level of: i) at least one gene product associated with each corresponding one or more ADC therapies, and at least one of the expression levels of: ii) at least one gene product associated with cell adhesion, and iii) one or more gene products associated with proliferation; (b) measuring the expression levels of one or more housekeeping genes in the same tumor tissue sample of step (a), wherein the one or more housekeeping genes comprise three genes selected from CIAO1, EIF2B1, HMBS, CTCF, GGNBP2, ITGB7, MYC and SLC4A1AP, and further normalizing the expression level of the at least one gene product associated with each of the one or more ADC therapies, the at least one gene product associated with cell adhesion, and one or more gene products associated with proliferation of step (a) to the three housekeeping genes to obtain normalized expression levels of the at least one gene product associated with each corresponding one or more ADC therapies, at least one gene product associated with cell adhesion, and one or more genes associated with proliferation; (c) if gene products of one or more genes associated with proliferation are measured and normalized, determining proliferation gene expression level by averaging the normalized expression levels of the one or more gene products associated with proliferation; (d) optionally, determining tumor cellularity in the same tumor tissue sample of steps (a) and (b); (e)(1) identifying a subject likely to benefit from the one or more ADC therapies when one or more of a calculated ADC Treatment Response Score (ADC TRS) surpass one or more corresponding predetermined thresholds, wherein each of the one or more ADC TRS is determined from: (i) the measured expression level of the at least one gene product associated with a corresponding ADC therapy, and at least two of: (ii) the measured expression level of the at least one gene product associated with cell adhesion, (iii) the determined proliferation gene expression level, and (iv) the determined tumor cellularity.

In some embodiments, the ADC TRS is determined by adding the expression level of a first gene product associated with a first ADC therapy to approximately 1.5 times the determined tumor cellularity and approximately ¼ of the determined proliferation gene expression level.

In some embodiments, the ADC Treatment Response Score is determined as follows:

$$ADC Treatment Response Score = 1 * [ Target Expression ] + 1.5 * [ Tumor Cellularity ] + \text{.25} * [ Proliferation Gene Expression ] ,$$

Wherein the [Target Expression], [Tumor Cellularity] and [Proliferation Gene Expression] values were previously z-score normalized and/or log 2 transformed.

In some embodiments, the expression product of the at least one gene associated with each of the one or more ADC therapies, the at least one gene product associate with cell adhesion, and one or more genes associated with proliferation are individually selected from a ribonucleic acid (RNA) and a protein. In some embodiments, the gene expression product of the at least one gene associated with each of the one or more ADC therapies, the at least one gene product associated with cell adhesion, and the one or more genes associated with proliferation is a protein, and measuring the expression level thereof requires making use of immunohistochemistry techniques. In some embodiments, the gene expression product of the at least one genes associated with each of the one or more ADC therapies, the at least one gene product associated with cell adhesion, and the one or more gene associated with proliferation is a RNA, and measuring the expression level thereof requires making use of RNA sequencing techniques, quantitative real-time polymerase chain reaction (qPCR), northern hybridization, microarray, or serial analysis of gene expression (SAGE). In some embodiments, the RNA sequencing techniques include shot-gun RNA-seq or full-length RNA-seq.

In some embodiments, each of the one or more ADC therapies comprise a monoclonal antibody, at least one functional fragment thereof or a bispecific antibody which targets at least one epitope of at least one antigen selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, CD142, CD25, GCC, MCP, FLT3, PTK7, IGF-1R, AG7, CD56, LY75, CD166, and CEACAM5. In some embodiments, the antibody or fragment thereof is conjugated directly or indirectly to a cytotoxic drug. In some embodiments, the cytotoxic drug is a DNA replication inhibitor selected from the group consisting of an alkylating agent, a DNA polymerase inhibitor, a nitrogen mustard and a topoisomerase inhibitor. In some embodiments, the antibody, the at least one functional fragment thereof, or the bispecific antibody is fused to a protein which is toxic to a cancer cell. In some embodiments, the cytotoxic drug is a topoisomerase inhibitor.

In some embodiments, the antibody comprises a bispecific antibody capable of targeting two epitopes of the same antigen or an epitope of two distinct antigens, wherein the same antigen or the two antigens are selected from the group consisting of SLC39A6, VTCN1, ERBB3, MET, ERBB2, TACSTD2, FOLR1, PVRL4, F3, SLC34A2, MSLN, B7-H3, B7-H4, 5T4, GPR20, AXL, TFR1, P79, EGFR, Integrin beta-6, ROR1, Globo H, CD142, CD25, GCC, MCP, FLT3, PTK7, IGF-1R, AG7, CD56, LY75, CD166, and CEACAM5.

In some embodiments, the one or more ADC therapies is selected from the group consisting of Mirvetuximab soravtansine, Tisotumab vedotin-tiftv, Trastuzumab deruxtecan, Enfortumab vedotin, Trastuzumab emtansine, STRO-002, PF-06804103, Cofetuzumab pelidotin, W0101, ZW49, ASN-004, XMT-1592, XMT-1536, BAT8001, ABGn-107, Lorvotuzumab mertansine, AVID100, B003, MEN1309, CX-2009, SAR408701, Anetumab ravtansine, Trastuzumab duocarmazine, MGC018, SYD1875, DS-7300a, U3-1402, DS-6157a, DS-1062a, MORAB-202, Enapotamab vedotin, BA3011, CX-2029, SGN-CD228A, Telisotuzumab vedotin, Disitimab vedotin, ALT-P7, MRG002, MRG003, OBI-999, SGN-B6A, VLS-101, Ladiratuzumab vedotin, Tisotumab vedotin, ARX788, FS-1502, A166, TR 1801-ADC, Camidanlumab tesirine, Serclutamab talirine, TAK-164, SHR-A1403, NBE-002, SKB-264, BDC-1001, SBT6050, BA3021, FOR-46, ABBV-011, ABBV-155, DP303c, GQ1001, BB-1701, and SHR-A1811. In some embodiments, the subject has or is suspected of having a cancer not approved for labeled use of the one or more ADC therapies.

In some embodiments, the tumor tissue sample is a formalin-fixed paraffin-embedded (FFPE) tumor tissue sample. In some embodiments, the tumor tissue sample contains at least 20% tumor content.

In some embodiments, the method further comprises the step (E) administering the at least one of the one or more ADC therapies to a subject identified in step (D)(1) as likely to respond to the one or more ADC therapies.

In some embodiments, the each of the one or more ADC TRS is determined without taking into account tumor cellularity.

In embodiments of the methods disclosed herein, the biological sample (i.e., sample) is any suitable sample type. In some embodiments, the sample is from plasma, blood, serum, saliva, sputum, stool, a tumor, cell free DNA, circulating tumor cell, or other biological sample. In some embodiments, the sample is a blood sample. In some embodiments, the biological sample is a tumor specimen. In some embodiments, the sample is from a subject having or at risk of having cancer. The type of cancer is not limited and may be any suitable cancer. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstram's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is lung or prostate cancer.

In some embodiments, the tumor tissue sample is or is suspected of containing bladder cancer, salivary gland cancer, endometrial cancer, ovarian cancer, cervical cancer, head and neck cancer, non-melanoma skin cancer, thyroid cancer, cancer of unknown primary, cancer of the central or peripheral nervous system, neuroendocrine tumor, melanoma, esophagogastric cancer, small bowel cancer, sarcoma, hepatobiliary cancer, pancreatic cancer, gastrointestinal stromal tumor, renal cell carcinoma, glioma, appendiceal cancer breast cancer, small cell lung cancer, prostate cancer, esophagogastric cancer, non-small cell lung cancer, pancreatic cancer, lymphoma or colorectal cancer.

In other aspects, the invention is directed to a method for selecting one or more antibody-drug conjugate (ADC) therapies among two or more ADC therapies identified as most beneficial to treat a cancer in a subject, the method comprising the steps of (A) measuring the expression level of at least one gene product associated with each of the one or more ADC therapies from a biological tissue sample obtained from the subject; (B) measuring in the same biological tissue sample of step (A), the expression level of one or both of: (i) at least one gene product associated with cell adhesion, and (ii) one or more gene products associated with proliferation, wherein if the expression level of more than one gene product associated with proliferation is measured, calculating therefrom an average of all expression levels of the measured gene products associated with proliferation, thereby obtaining a proliferation gene expression level; (C) optionally, determining tumor cellularity in the same tumor tissue sample of steps (A) and (B); (D)(1) calculating an ADC Treatment Response Score (ADC TRS) for each of the two or more ADC therapies, and determining each of the two or more ADC TRS surpass a predetermined threshold associated with beneficial patient treatment outcome, wherein each of the one or more ADC TRS is determined from: (i) the measured expression level of the at least one gene product associated with a corresponding ADC therapy, and at least two of: (ii) the measured expression level of at least one gene product associated with cell adhesion, (iii) the determined proliferation gene expression level, and (iv) the determined tumor cellularity; (D)(2) wherein if ADC TRS associated with two or more ADC therapies surpass a predetermined threshold associated with a beneficial patient treatment outcome, ranking the at least two ADC TRS by the value by which each ADC Treatment Response Score exceeds the predetermined threshold, and selecting the highest ranked ADC therapy for administration to a subject.

In some embodiments, the method further comprises step (E) of administering to the subject the selected highest ranked ADC therapy. In some embodiments, step (E) further comprises administering to the subject at least one other lower ranked ADC exceeding the predetermined threshold in combination with the highest ranked ADC therapy. In some embodiments, step (E) does not include administering another ADC therapy in combination with the highest ranked ADC therapy.

In some embodiments, two, three, four, five, six, seven, eight, nine, ten, eleven, twelve, thirteen, fourteen, or fifteen or more ADC therapies are identified as therapies to which a cancer patient is likely to respond. In some embodiments two or more, or three or more ADC therapies are administered to the patient sequentially or concurrently. In some embodiments, the patient has already been treated with a first line therapy, a second line therapy, a third therapy, or a fourth line therapy, prior to being administered the one or more ADC therapies identified as a therapy to which the cancer patient is likely to respond.

In some embodiments, a subject is identified as likely to respond to the one or more ADC therapies, when the measured expression level of the at least one gene product associated with each of the one or more corresponding ADC therapies, the determined proliferation gene expression level, and the determined tumor cellularity are all higher than corresponding median levels of the at least one gene product expression, proliferation gene expression and median tumor cellularity obtained from tumor tissue samples from the same first cohort of subjects, while the measured expression level of the at least one gene product associated with cell adhesion, such as the PVR gene product, is below a median expression level of the at least one corresponding gene product associated with cell adhesion, such as the PVR gene product, in the same first cohort of subjects. In other embodiments, the measured expression level of the at least one gene product associated with each of the one or more ADC therapies, the determined proliferation gene expression level, and the determined tumor cellularity may be 10%, 15%, 20%, 25%, 30%, 35%, 45%, 50%, 55%, 60%, 65%, 70%, 75%, 80%, 85%, 90%, 95%, 100%, 125%, 150%, 175%, 200%, 250%, 300% or higher than corresponding median levels of the at least one gene product expression, proliferation gene expression and median tumor cellularity obtained from tumor tissue samples from the same first cohort of subjects. In some other embodiments, the measured expression level of the at least one gene product associated with each of the one or more ADC therapies, the determined proliferation gene expression level, and the determined tumor cellularity may be 1.1 fold, 1.2 fold, 1.25 fold, 1.5 fold, 1.75 fold, 1.9 fold, 2 fold, 3 fold, 4 fold, 5 fold or more higher than corresponding median levels of the at least one gene product expression, proliferation gene expression and median tumor cellularity obtained from tumor tissue samples from the same first cohort of subjects.

In some embodiments, the subject is identified as likely to respond to one or more ADC therapies when the measured expression level of the at least one gene product associated with each of the one or more ADC therapies, the determined proliferation gene expression level, and/or the determined tumor cellularity fall into the highest quartile of the one or more gene products expression levels, proliferation gene expression level and/or tumor cellularity values obtained from tumor tissue samples from the same first cohort of subjects, while the expression level of the at least one gene product associated with cell adherence, such as the PVR gene product, falls into the lowest quartile of expression levels the corresponding at least one gene product associated with cell adherence, such as the PVR gene product, from tumor samples obtained from the same first cohort of subjects. In some embodiments, the subject is identified as likely to respond to the one or more ADC therapies when one or more of the measured expression levels of the at least one gene product, the determined proliferation gene expression level, and/or the determined tumor cellularity fall into the highest top 30%, top 25%, top 20%, top 15%, top 10%, top 5%, top 3% or top 1% of the at least one gene product expression level, proliferation gene expression level and/or tumor cellularity values obtained from tumor tissue samples from the same first cohort of subjects. In some embodiments, the subject is identified as likely to response to the one or more ADC therapies when the expression level of the at least one gene product associated with cell adhesion, such as the PVR gene product, falls into the lowest 30%, lowest 25%, lowest 20%, lowest 15%, lowest 10%, lowest 5%, lowest 3%, or lowest 1% of the expression level of the corresponding at least one gene product associated with cell adhesion, such as the PVR gene product, from tumor tissue samples from the same first cohort of subjects.

In another aspect, the invention is directed to a computer-implemented method for selecting a patient presenting with a solid cancerous tumor for treatment by one or more antibody-drug conjugate (ADC) therapies, wherein the method comprises the steps of: (A) receiving a measured expression level of at least one gene product associated with each of the one or more ADC therapies from a biological tissue sample obtained from the tumor tissue of the patient; (B)(1) receiving an expression level of one or both of: (i) at least one gene product associated with cell adhesion, such as the PVR gene product, and (ii) one or more gene products associated with proliferation, wherein the expression levels of (i) and (ii) are measured in the same biological tumor tissue sample of step (A), (B)(2) if the measured expression level of the one or more gene products associated with proliferation is received, calculating therefrom, with a computer, an average of all expression levels of the received measured gene products associated with proliferation, thereby obtaining a proliferation gene expression level; (C) optionally, receiving an indication of tumor cellularity of the same tumor tissue sample of steps (A) and (B); (D)(1) identifying a subject likely to respond to the one or more ADC therapies when either: (i) one or more of a calculated ADC Treatment Response Score (ADC TRS) surpass one or more corresponding predetermined thresholds, wherein each of the one or more ADC TRS is determined from: (1) the received measured expression level of the at least one gene product associated with a corresponding ADC therapy, and at least two of: (2) the received measured expression level of the at least one gene product associated with cell adhesion, such as the PVR gene product, (3) the calculated proliferation gene expression level from the received expression levels of one or more gene products associated with proliferation, and (4) the received indication of tumor cellularity; and (E) selecting the patient identified as likely to respond to the one or more ADC therapies for treatment therewith, wherein at least steps (A)-(D)(1)(i) are performed with a suitably programmed computer.

In some embodiments, step (E) comprises selecting the patient to receive treatment with the one or more ADC therapies as part of a clinical trial. In some embodiments, the clinical trial is a basket trial. In some embodiments, the method further comprises step (F) treating the selected patient with the one or more ADC therapies determined to likely induce a response in the patient.

### Methods of Targeted Treatment to Subjects Likely to Respond Thereto

In some aspects, the invention is directed to a method of treating a cancer in a subject determined likely to respond to one or more antibody-drug conjugate (ADC) therapies, the method comprising the steps of: (A) measuring the expression level of at least one gene product associated with each of the one or more ADC therapies from a biological tissue sample obtained from the subject; (B) measuring in the same biological tissue sample of step (A), the expression level of one or both of: (i) at least one gene product associated with cell adhesion, and (ii) one or more gene products associated with proliferation, wherein if the expression level of the one or more gene products associated with proliferation is measured, calculating therefrom an average of all expression levels of the measured gene products associated with proliferation, thereby obtaining a proliferation gene expression level; (C) optionally, determining tumor cellularity in the same tumor tissue sample of steps (A) and (B); (D)(1) identifying a subject likely to respond to the one or more ADC therapies when one or more of a calculated ADC Treatment Response Score (ADC TRS) surpass one or more corresponding predetermined thresholds, wherein each of the one or more ADC TRS is determined from: (i) the measured expression level of the at least one gene product associated with a corresponding ADC therapy, and at least two of: (ii) the measured expression level of the at least one gene product associated with cell adhesion, (iii) the determined proliferation gene expression level, and (iv) the determined tumor cellularity; and (E) administering at least one of the one or more ADC therapies to the subject identified as likely to respond to the one or more ADC therapies.

In some embodiments, the step of measuring the expression level of one or more gene products associated with proliferation comprises measuring the expression levels of a gene product of one or more genes which are cell cycle regulated and pertain to DNA replication, mitotic processes/phases, spindle assembly, tubulin, mitotic surveillance, cell adhesion, chromosome metabolism, and histone formation. In some embodiments genes with gene products associated with proliferation are one or more genes selected from the group consisting of BIRC5, BRCA1, BRCA2, BUB1, BUB1B, CCNA2, CCNB1, CCNB2, CCNE1, CCNE2, CCNF, CCNG2, CDC2, CDC20, CDC25A, CDC25B, CDC25C, CDC45L, CDC6, CDC7, CDCKN1A p21, CDCKN3, CDK4, CDKN1C, CDKN2A, CDKN2C, CDKN2D p19, CDKN3, CENPA, CENPE, CENPF, CHAF1A, CHK1, CKS1, CKS2, CKS2, DHFR, DHFR, E2-EPF, E2F1, E2F3, E2F5, FEN1, FOXM1, KNSL2, KNSL5, KPNA2, LMNB2, MAD2, MAD2L1, MAPK13, MCM2, MCM3, MCM4, MCM5, MCM6, MK167, MNAT1, MSH2, MYBL2, NASP, NEK2, NPAT, ORC1, PA2G4, PCNA, PES1, PKMYT1, PLK, PRIM1, PTTG1, RAB6KIFL, RACGAP1, RAD51, RAD54L, RAN RAN, RFC4, RPA3, RRM1, RRM2, SLBP, STK12, STK15, STK18, STK6, TOP2A, TROAP, TUBB, TYMS, UBE2C, and UNG. In some embodiments, the step of measuring the expression level of one or more gene products associated with proliferation comprises measuring the expression level of one, two or all three MYBL2, TOP2A, and/or UBE2C gene products. In certain embodiments, the step of measuring the expression level of one or more gene products associated with proliferation comprises measuring the expression level of TOP2A, and UBE2C gene products.

The inventors have recently discovered that adding an additional variable enhanced predictive power of the method in predicting a given patient response to ADC therapy. Thus, in some embodiments, the ADC TRS is determined by at least the combination of an expression level of at least one gene product associated with a corresponding ADC therapy, one or both of tumor cellularity and proliferation gene expression level, as well as the expression level of one additional gene product. In some embodiments, the predictive power of the ADC TRS can be enhanced by the addition of a variable comprising an expression level of at least one gene product associated with cell adhesion. Consequently, in certain embodiments, the ADC TRS is determined by one gene product associated with a corresponding ADC therapy, a proliferation gene expression level, as well as the expression level of at least one gene product associated with cell adhesion.

In some embodiments, both the determined proliferation gene expression level and the expression level of the at least one gene product associated with a corresponding ADC therapy are positively associated with a likelihood that a patient will respond to the corresponding ADC therapy, and the expression level of the at least one gene product associated with cell adhesion is negatively associated with a likelihood that a patient will respond to the same corresponding ADC therapy. In some embodiments, the expression level of the at least one gene product associated with a corresponding ADC therapy is weighted by a factor of approximately 0.3 to 0.7, approximately 0.35 to 0.65, approximately 0.4 to approximately 0.5, or approximately 0.4, 0.45, 0.48, 0.50, 0.55, 0.60, 0.65, 0.68, or 0.7. In some embodiments, the expression level of the at least one gene product associated with cell adhesion is weighted by a factor of approximately −1.5 to −0.5, −1.25 to −0.75, −1.1 to −0.8, −1 to −0.8, or approximately −1.05, −1.0, −0.95, −0.90 or −0.85. In some embodiments, the determined proliferation gene expression level is weighted by a factor of approximately 0.15 to 0.60, approximately 0.20 to 0.40, or approximately 0.45, 0.40, 0.38, 0.35, 0.30, or 0.25.

According to one aspect of the invention, the gene product associated with cell adhesion is a gene product providing an expression level in a cohort of cancer patients which is negatively correlated with the objective response rate to at least one ADC therapy in this same cohort of cancer patients. The statistical methods used for determining the negative correlation between objective response rates and expression level of a gene product associated with cell adhesion is not particularly limited, and will be within the ordinary skill of one in this field, and includes those methods well known in the field, such as Spearman correlation or Pearson correlation. In some embodiments, the correlation coefficients associated with each cell adhesion associated gene product obtained by such analysis can be ranked and selected based upon its proximity to −1. Thus, in some embodiments, a cancer patient is deemed likely to respond to at least one ADC therapy only when the expression of at least one gene product associated with cell adhesion is reduced compared to a reference level. In some embodiments, the reference level is the mean expression level of the same gene product associated with cell adhesion in cancer subjects of an ADC therapy treatment cohort. In some embodiments, a cancer patient is deemed likely to respond to at least one ADC therapy only when the expression level of at least one gene product associated with cell adhesion falls at least two standard deviations below a mean expression level of the same gene product in cancer subjects of an ADC therapy treatment cohort.

In some embodiments, the gene product associated with cell adhesion is a gene product which impacts a cellular component of cell adhesion comprising an adherens junction, an anchoring junction, a cell-substrate adherens junction, a cell-substrate junction, and a focal adhesion. In certain embodiments the gene product associated with cell adhesion impacts at least two cellular components selected from the group consisting of an adherens junction, an anchoring junction, a cell-substrate adherens junction, a cell-substrate junction, and a focal adhesion. In some embodiments, the gene product associated with cell adhesion impacts at least three, at least four or all five of the cellular components. In some embodiments, the gene product associated with cell adhesion is chosen both because of its negative correlation to objective response rates and because it impacts all five cellular components selected from the group consisting of an adherens junction, an anchoring junction, a cell-substrate adherens junction, a cell-substrate junction, and a focal adhesion. By way of non-limiting example, the above Table 1 outlines genes which provide expression levels of gene products associated with cell adhesion which are negatively correlated with an objective response rate in cancer subjects of a cohort and the specific cellular components of cell adhesion impacted by their respective expression levels as discussed above.

In some embodiments, the step of measuring the expression level of at least one gene product associated with cell adhesion comprises measuring the expression level of a gene product of at least one gene selected from the group consisting of ATP2A2, BAIAP2, CD151, CHP1, CYFIP1, CYTH3, DAG1, DSC2, GIT1, HSP90B1, HSPA5, LIMK1, MAPK1, PACSIN2, PDIA3, PVR, REXO2, RPL22, RPLP1, RPLP2, RPS11, RPS16, RPS5, SDCBP SNAP23, SNTB1, and SRP68. In certain embodiments, the expression levels of two or more gene products of two or more genes selected from the group consisting of ATP2A2, BAIAP2, CD151, CHP1, CYFIP1, CYTH3, DAG1, DSC2, GIT1, HSP90B1, HSPA5, LIMK1, MAPK1, PACSIN2, PDIA3, PVR, REXO2, RPL22, RPLP1, RPLP2, RPS11, RPS16, RPS5, SDCBP SNAP23, SNTB1, and SRP68 are measured. When expression levels of two or more of the gene products associated with cell adhesion are measured, an average expression level may be calculated to provide a cell adhesion gene product expression level. Accordingly, in some embodiments, a cancer patient is deemed likely to respond to an ADC therapy only when the cell adhesion gene product expression level is reduced compared to a reference level. Prior to averaging the multiple expression levels of gene products associated with cell adhesion, each gene product expression level may be normalized and/or log 2 transformed. To facilitate the normalization of each gene product expression level, a mean and standard deviation of the corresponding gene product expression levels in a cancer patient cohort can be used. Thus, in some embodiments, a patient is identified as likely to respond to an ADC therapy only when a normalized cell adhesion gene product expression level is less than zero.

In one embodiment, the step of measuring the expression level of at least one gene product associated with cell adhesion comprises measuring the expression of a PVR gene product. In addition, at least one, two, three, four, five, ten, fifteen, twenty, twenty-five, or thirty additional gene products associated with cell adhesion can be measured along with the PVR gene product. In some embodiments, the step of measuring the expression level of at least one gene product associated with cell adhesion consists of measuring the expression level of a single gene product associated with cell adhesion. In some embodiments, only the expression of a PVR gene product is measured. In some embodiments, only the expression of a SRP68 gene product is measured. In some embodiments, only the expression of a SNTB1 gene product is measured. In some embodiments, only the expression of a SNAP23 gene product is measured. In some embodiments, only the expression of a SDCBP gene product is measured. In some embodiments, only the expression of a RPS5 gene product is measured. In some embodiments, only the expression of a RPS16 gene product is measured. In yet another embodiment, only the expression of a RPS11 gene product is measured. In some embodiments, only the expression of a RPLP2 gene product is measured. In some embodiments, only the expression of a RPLP1 gene product is measured. In some embodiments, only the expression of a REXO2 gene product is measured. In some embodiments, only the expression of a PDIA3 gene product is measured. In some embodiments, only the expression of a PACSIN2 gene product is measured. In some embodiments, only the expression of a MAPK1 gene product is measured. In some embodiments, only the expression of a LIMK1 gene product is measured. In some embodiments, only the expression of a HSPA5 gene product is measured. In some embodiments, only the expression of a HSP90B1 gene product is measured. In some embodiments, only the expression of a GIT1 gene product is measured. In some embodiments, only the expression of a DSC2 gene product is measured. In some embodiments, only the expression of a DAG1 gene product is measured. In some embodiments, only the expression of a CYTH3 gene product is measured. In some embodiments, only the expression of a CYFIP1 gene product is measured. In some embodiments, only the expression of a CHP1 gene product is measured. In some embodiments, only the expression of a CD151 gene product is measured. In some embodiments, only the expression of a BAIAP2 gene product is measured. In some embodiments, only the expression of a ATP2A2 gene product is measured.

In some embodiments, the ADC TRS is determined by at least the combination of the expression level of the at least one gene product associated with a corresponding ADC therapy, the expression level of the at least one gene product associated with cell adhesion, the determined proliferation gene expression level, and tumor cellularity. In some embodiments, both the determined proliferation gene expression level, the tumor cellularity, and the expression level of the at least one gene product associated with a corresponding ADC therapy are positively associated with a likelihood that a patient will respond to the corresponding ADC therapy, and the expression level of the at least one gene product associated with cell adhesion is negatively associated with a likelihood that a patient will respond to the same corresponding ADC therapy.

In some embodiments, the expression level of the at least one gene product associated with a corresponding ADC therapy is weighted by a factor of approximately 0.30 to 0.70, approximately 0.35 to 0.65, approximately 0.4 to approximately 0.5, or approximately 0.40, 0.45, 0.48, or 0.50. In some embodiments, the expression level of the at least one gene product associated with cell adhesion is weighted by a factor of approximately −1.5 to −0.5, −1.25 to −0.75, −1.1 to 0.8, or approximately −1.05, −1.0, −0.95, −0.90 or −0.85. In some embodiments, the determined proliferation gene expression level is weighted by a factor of approximately 0.75 to 0.35, 0.65 to 0.40, 0.60 to 0.50, or approximately 0.65, 0.60, 0.55, 0.50, or 0.45. In some embodiments, the tumor cellularity is weighted by a factor of approximately 0.25 to 0.01, 0.20 to 0.02, 0.17 to 0.04, or approximately 0.09, 0.08, 0.07, 0.06, or 0.05. In some embodiments, the expression level of the at least one gene product associated with a corresponding ADC therapy is weighted by a factor of approximately 0.45, the expression level of the at least one gene product associated with cell adhesion is weighted by a factor of approximately −0.98, the determined proliferation gene expression level is weighted by a factor of approximately 0.55, and the tumor cellularity is weighted by a factor of approximately 0.07.

In some embodiments, each of the one or more ADC TRS is determined by further taking into account a bias variable, wherein the bias variable is a static offset tuned to yield biomarker frequencies that match published objective response rates in clinical trials. In some embodiments, the bias variable is weighted by a factor of approximately −0.65 to −0.1, −0.55 to −0.15, −0.35 to 0.20, or approximately −0.22, −0.23, −0.24, −0.25, −0.25, −0.26, −0.27, or −0.28. In some embodiments the bias variable is weighted by a factor of approximately −0.26.

As discussed above, the ADC TRS is determined by multiple variables that are weighted individually based upon their influence on the probability that a subject is likely to response to an ADC therapy. When these variables are combined, a score is obtained which indicates a subject is likely to response to an ADC therapy when it surpasses a certain predetermined threshold. In some embodiments, the predetermined threshold is zero, and an ADC TRS which indicates the subject is likely to respond to an ADC therapy is an ADC TRS with a value greater than zero.

In some embodiments, the predetermined threshold is set to a percentile of ranked ADC Treatment Response Scores determined from tumor tissue samples of a first cohort of subjects utilizing at least the expression level of the at least one gene product associated with a first corresponding ADC therapy, wherein each percentile corresponds to a percentage greater than a percentage of subjects of the first cohort which do not respond to the same first corresponding ADC therapy.

In some embodiments, the predetermined thresholds are set to percentiles of ranked ADC Treatment Response Scores determined from tumor tissue samples of a first and a second cohort of subjects utilizing at least the expression level of at least a first and a second gene product associated with a first and second corresponding ADC therapy, wherein each percentile corresponds to a percentage greater than a percentage of subjects of the first and second cohort which do not respond to the at least first and second corresponding ADC therapies.

In some embodiments, the first cohort of subjects is a pan-cancer cohort or a matched tumor-type cohort. In some embodiments, the first cohort is a matched tumor-type cohort. The matched tumor-type cohort is not particularly limited and includes those tumors/cancer known to those skilled in the art and disclosed herein. In some embodiments, the matched tumor-type is appendiceal cancer, bladder cancer, breast cancer, cervical cancer, CNS and PNS cancer, colorectal cancer, endometrial cancer, esophagogastric cancer, gastrointestinal stromal tumor, glioma, head and neck cancer, hepatobiliary cancer, lymphoma, melanoma, neuroendocrine tumor, non-small cell lung cancer, ovarian cancer, prostate cancer, renal cell carcinoma, salivary gland cancer, sarcoma, non-melanoma skin cancer, small bowel cancer, small cell lung cancer, or thyroid cancer.

In still other embodiments, the first cohort is a pan-cancer cohort. Such pan-cancer cohorts may contain 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more different cancers represented in the cohort. In certain embodiments, said cohort contains three or more cancers represented from the group consisting of appendiceal cancer, bladder cancer, breast cancer, cervical cancer, CNS and PNS cancer, colorectal cancer, endometrial cancer, esophagogastric cancer, gastrointestinal stromal tumor, glioma, head and neck cancer, hepatobiliary cancer, lymphoma, melanoma, neuroendocrine tumor, non-small cell lung cancer, ovarian cancer, prostate cancer, renal cell carcinoma, salivary gland cancer, sarcoma, non-melanoma skin cancer, small bowel cancer, small cell lung cancer, and thyroid cancer.

In some embodiments the first cohort is a clinical trial cohort comprising cancer patients who are receiving at least one antibody-drug conjugate therapy. In some embodiments, the patients are selected for inclusion in the clinical trial cohort on account of having an advanced cancer and displaying expression of one target of a candidate antibody-drug conjugate therapy. In some embodiments the advanced cancer has been radiologically confirmed to have metastasized or has been determined to be relapsing or refractory to a prior treatment with a chemotherapeutic drug. In some embodiments, the patient has an advanced solid tumor. In a preferred embodiment the cohort comprises at least 50 patients, at least 75 patients, at least 100 patients, at least 125 patients, at least 150 patients, at least 175 patients, at least 200 patients, at least 250 patients, at least 300 patients, at least 400 patients, at least 500 patients or more. In some embodiments, the cohort has at least 200 patients. In some embodiments, the clinical trial cohort is a basket trial/pan-cancer cohort. In some embodiments, the first cohort is a tumor specific cohort.

In some embodiments, the ADC Treatment Response Score is determined by the combination of the expression level of at least one gene product associated with each of the one or more corresponding ADC therapies, a determined proliferation gene expression level, and tumor cellularity. In some embodiments, the predetermined threshold is set to the percentile of ranked ADC Treatment Response Scores determined from tumor tissue samples of a second cohort of subjects, which percentile corresponds to a percentage of subjects of the second cohort which do not respond to the one or more ADC therapies.

In some embodiments, the second cohort of subjects is a pan-cancer cohort or a matched tumor-type cohort. In some embodiments, the second cohort is a matched tumor-type cohort. The matched tumor-type cohort is not particularly limited and includes those tumors/cancer known to those skilled in the art and disclosed herein. In some embodiments, the matched tumor-type is appendiceal cancer, bladder cancer, breast cancer, cervical cancer, CNS and PNS cancer, colorectal cancer, endometrial cancer, esophagogastric cancer, gastrointestinal stromal tumor, glioma, head and neck cancer, hepatobiliary cancer, lymphoma, melanoma, neuroendocrine tumor, non-small cell lung cancer, ovarian cancer, prostate cancer, renal cell carcinoma, salivary gland cancer, sarcoma, non-melanoma skin cancer, small bowel cancer, small cell lung cancer, or thyroid cancer.

In still other embodiments, the second cohort is a pan-cancer cohort. Such pan-cancer cohorts may contain 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more different cancers represented in the cohort. In certain embodiments, said cohort contains three or more cancers selected from the group consisting of cancer appendiceal cancer, bladder cancer, breast cancer, cervical cancer, CNS and PNS cancer, colorectal cancer, endometrial cancer, esophagogastric cancer, gastrointestinal stromal tumor, glioma, head and neck cancer, hepatobiliary cancer, lymphoma, melanoma, neuroendocrine tumor, non-small cell lung cancer, ovarian cancer, prostate cancer, renal cell carcinoma, salivary gland cancer, sarcoma, non-melanoma skin cancer, small bowel cancer, small cell lung cancer, and thyroid cancer.

In some embodiments, the first cohort of subjects and the second cohort of subjects are the same cohort of subjects.

In some embodiments, gene expression level of the at least one gene product associated with each one or more ADC therapies, the expression level of the at least one gene product associated with cell adhesion, and the tumor cellularity are log 2 transformed and/or Z score normalized prior to step (D)(1), and the expression level of the one or more gene products associated with proliferation are log 2 transformed and/or Z score normalized prior to the step of averaging expression levels of gene products associated with proliferation to obtain the proliferation gene expression level in step (B).

As discussed above, it is preferable that the gene expression level of the one or more gene products associated with ADC therapies, the gene product associated with cell adhesion, proliferation as well as the tumor cellularity values are subjected to normalization to minimize the impact of outliers and to facilitate enhanced predictive power of the method. In some embodiments, the normalization is Z-score normalization. In some embodiments the normalization is min-max normalization. In some embodiments, the normalization is Z-score normalization based upon data from a from a large number of solid tumors, such as data obtained from at least 5,000, at least 10,000, at least 15,000, or at least 20,000 distinct solid tumor samples. In some embodiments, Z-score normalization is based upon data from more than 15,000 solid tumors in the Strata Trial (NCT03061305).

In some embodiments, the method further comprises measuring the expression level of at least one housekeeping gene selected from CIAO1, EIF2B1, and HMBS in the tumor tissue sample, and normalizing the at least one gene product associated with the one or more ADC therapies, the at least one gene product associated with cell adhesion, and the one or more gene products associated with proliferation to the at least one housekeeping gene expression to obtain normalized expression levels of the at least one gene product associated with each of the one or more ADC therapies, the at least one gene product associated with cell adhesion, and the one or more gene products associated with proliferation. Thus, in some embodiments, the invention is directed to a method for treating a cancer in a subject likely to respond to one or more antibody-drug conjugate (ADC) therapies, comprising: (a) measuring, in a tumor tissue sample obtained from the subject, the expression level of: i) at least one gene product associated with each corresponding one or more ADC therapies, and at least one of the expression levels of: ii) at least one gene product associated with cell adhesion, and iii) one or more gene products associated with proliferation; (b) measuring the expression levels of one or more housekeeping genes in the same tumor tissue sample of step (a) and further normalizing the expression level of the at least one gene product associated with each of the one or more ADC therapies, the at least one gene product associated with cell adhesion, and one or more gene products associated with proliferation of step (a) against the expression levels of the one or more housekeeping genes to obtain normalized expression levels of the at least one gene product associated with each corresponding one or more ADC therapies, a gene product associated with cell adhesion, and one or more genes associated with proliferation; (c) if gene products of one or more genes associated with proliferation are measured and normalized, determining proliferation gene expression level by averaging the normalized expression levels of the one or more gene products associated with proliferation; (d) optionally, determining tumor cellularity in the same tumor tissue sample of steps (a) and (b); (e)(1) identifying a subject likely to benefit from the one or more ADC therapies when one or more of a calculated ADC Treatment Response Score (ADC TRS) surpass one or more corresponding predetermined thresholds, wherein each of the one or more ADC TRS is determined from: (i) the measured expression level of the at least one gene product associated with a corresponding ADC therapy, and at least two of: (ii) the measured level of the at least one gene product associated with cell adhesion, (iii) the determined proliferation gene expression level, and (iv) the determined tumor cellularity; and (f) administering an effective amount of the one or more ADC therapies to a subject identified as likely to benefit from the one or more ADC therapies.

In some embodiments, the ADC TRS is determined by adding the expression level of a first gene product associated with a first ADC therapy to approximately 1.5 times the determined tumor cellularity and approximately ¼ of the determined proliferation gene expression level.

In some embodiments, the ADC Treatment Response Score is determined as follows:

$$ADC Treatment Response Score = 1 * [ Target Expression ] + 1.5 * [ Tumor Cellularity ] + \text{.25} * [ Proliferation Gene Expression ] ,$$

Wherein the [Target Expression], [Tumor Cellularity] and [Proliferation Gene Expression] values were previously z-score normalized and/or log 2 transformed.

In some embodiments, the expression product of the at least one gene associated with each of the one or more ADC therapies, the at least one gene product associate with cell adhesion, and one or more genes associated with proliferation are individually selected from a ribonucleic acid (RNA) and a protein. In some embodiments, the gene expression product of the at least one gene associated with each of the one or more ADC therapies, the at least one gene product associated with cell adhesion, and the one or more genes associated with proliferation is a protein, and measuring the expression level thereof requires making use of immunohistochemistry techniques. In some embodiments, the gene expression product of the at least one gene associated with each of the one or more ADC therapies, the at least one gene product associated with cell adhesion, and the one or more gene associated with proliferation is a RNA, and measuring the expression level thereof requires making use of RNA sequencing techniques or quantitative real-time polymerase chain reaction (qPCR).

In some embodiments, each of the one or more ADC therapies comprise a monoclonal antibody, at least one functional fragment thereof or a bispecific antibody which targets at least one epitope of at least one antigen selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, CD142, CD25, GCC, MCP, FLT3, PTK7, IGF-1R, AG7, CD56, LY75, CD166, and CEACAM5. In some embodiments, the antibody or fragment thereof is conjugated directly or indirectly to a cytotoxic drug. In some embodiments, the cytotoxic drug is a DNA replication inhibitor selected from the group consisting of an alkylating agent, a DNA polymerase inhibitor, a nitrogen mustard and a topoisomerase inhibitor. In some embodiments, the antibody, the at least one functional fragment thereof, or the bispecific antibody is fused to a protein which is toxic to a cancer cell. In some embodiments, the cytotoxic drug is a topoisomerase inhibitor.

In some embodiments, the antibody comprises a bispecific antibody capable of targeting two epitopes of the same antigen or an epitope of two distinct antigens, wherein the same antigen or the two antigens are selected from the group consisting of SLC39A6, VTCN1, ERBB3, MET, ERBB2, TACSTD2, FOLR1, PVRL4, F3, SLC34A2, MSLN, B7-H3, B7-H4, 5T4, GPR20, AXL, TFR1, P79, EGFR, Integrin beta-6, ROR1, Globo H, CD142, CD25, GCC, MCP, FLT3, PTK7, IGF-1R, AG7, CD56, LY75, CD166, and CEACAM5.

In some embodiments, the one or more ADC therapies is selected from the group consisting of Mirvetuximab soravtansine, Tisotumab vedotin-tiftv, Trastuzumab deruxtecan, Enfortumab vedotin, Trastuzumab emtansine, STRO-002, PF-06804103, Cofetuzumab pelidotin, W0101, ZW49, ASN-004, XMT-1592, XMT-1536, BAT8001, ABGn-107, Lorvotuzumab mertansine, AVID100, B003, MEN1309, CX-2009, SAR408701, Anetumab ravtansine, Trastuzumab duocarmazine, MGC018, SYD1875, DS-7300a, U3-1402, DS-6157a, DS-1062a, MORAB-202, Enapotamab vedotin, BA3011, CX-2029, SGN-CD228A, Telisotuzumab vedotin, Disitimab vedotin, ALT-P7, MRG002, MRG003, OBI-999, SGN-B6A, VLS-101, Ladiratuzumab vedotin, Tisotumab vedotin, ARX788, FS-1502, A166, TR 1801-ADC, Camidanlumab tesirine, Serclutamab talirine, TAK-164, SHR-A1403, NBE-002, SKB-264, BDC-1001, SBT6050, BA3021, FOR-46, ABBV-011, ABBV-155, DP303c, GQ1001, BB-1701, and SHR-A1811. In some embodiments, the subject has or is suspected of having a cancer not approved for labeled use of the one or more ADC therapies.

In some embodiments, the tumor tissue sample is a formalin-fixed paraffin-embedded (FFPE) tumor tissue sample. In some embodiments, the tumor tissue sample contains at least 20% tumor content.

In some embodiments, the each of the one or more ADC TRS is determined without taking into account tumor cellularity.

In embodiments of the methods disclosed herein, the biological sample (i.e., sample) is any suitable sample type. In some embodiments, the sample is from plasma, blood, serum, saliva, sputum, stool, a tumor, cell free DNA, circulating tumor cell, or other biological sample. In some embodiments, the sample is a blood sample. In some embodiments, the biological sample is a tumor specimen. In some embodiments, the sample is from a subject having or at risk of having cancer. The type of cancer is not limited and may be any suitable cancer. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstram's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is lung or prostate cancer.

In some embodiments, the tumor tissue sample is or is suspected of containing bladder cancer, salivary gland cancer, endometrial cancer, ovarian cancer, cervical cancer, head and neck cancer, non-melanoma skin cancer, thyroid cancer, cancer of unknown primary, cancer of the central or peripheral nervous system, neuroendocrine tumor, melanoma, esophagogastric cancer, small bowel cancer, sarcoma, hepatobiliary cancer, pancreatic cancer, gastrointestinal stromal tumor, renal cell carcinoma, glioma, appendiceal cancer breast cancer, small cell lung cancer, prostate cancer, esophagogastric cancer, non-small cell lung cancer, pancreatic cancer, lymphoma or colorectal cancer.

In other aspects, the invention is directed to a method for selecting one or more antibody-drug conjugate (ADC) therapies among two or more ADC therapies identified as most beneficial to treat a cancer in a subject, the method comprising the steps of (A) measuring the expression level of at least one gene product associated with each of the one or more ADC therapies from a biological tissue sample obtained from the subject; (B) measuring in the same biological tissue sample of step (A), the expression level of one or both of: (i) at least one gene product associated with cell adhesion, and (ii) one or more gene products associated with proliferation, wherein if the expression level of more than one gene product associated with proliferation is measured, calculating therefrom an average of all expression levels of the measured gene products associated with proliferation, thereby obtaining a proliferation gene expression level; (C) optionally, determining tumor cellularity in the same tumor tissue sample of steps (A) and (B); (D)(1) calculating an ADC Treatment Response Score (ADC TRS) for each of the two or more ADC therapies, and determining each of the two or more ADC TRS surpass a predetermined threshold associated with beneficial patient treatment outcome, wherein each of the one or more ADC TRS is determined from: (i) the measured expression level of the at least one gene product associated with a corresponding ADC therapy, and at least two of: (ii) the measured expression level of at least one gene product associated with cell adhesion, (iii) the determined proliferation gene expression level, and (iv) the determined tumor cellularity; (D)(2) wherein if ADC TRS associated with two or more ADC therapies surpass a predetermined threshold associated with a beneficial patient treatment outcome, ranking the at least two ADC TRS by the value by which each ADC Treatment Response Score exceeds the predetermined threshold, and administering the highest ranked ADC therapy to a subject.

In some embodiments, the one or more ADC therapies comprise at least two therapies, and step (D)(1) comprises calculating at least two ADC treatment response scores, wherein if the subject is identified likely to respond to at least two ADC therapies, the method further comprises a step between steps (D)(1) and (E) comprising step (D)(2) of ranking the at least two ADC Treatment Response Scores by the value by which each ADC Treatment Response Score exceeds the predetermined threshold, and in step (E) administering to the subject at least the highest ranked ADC therapy. In some embodiments, step (E) further comprises administering to the subject at least one other lower ranked ADC exceeding the predetermined threshold in combination with the highest ranked ADC therapy. In some embodiments, step (E) does not include administering another ADC therapy in combination with the highest ranked ADC therapy.

In some embodiments, two, three, four, five, six, seven, eight, nine, ten, eleven, twelve, thirteen, fourteen, or fifteen or more ADC therapies are identified as therapies to which a cancer patient is likely to respond. In some embodiments two or more, or three or more ADC therapies are administered to the patient sequentially or concurrently. In some embodiments, the patient has already been treated with a first line therapy, a second line therapy, a third therapy, or a fourth line therapy, prior to being administered the one or more ADC therapies identified as a therapy to which the cancer patient is likely to respond.

In some embodiments, a subject is identified as likely to respond to the one or more ADC therapies, when the measured expression level of the at least one gene product associated with each of the one or more corresponding ADC therapies, the determined proliferation gene expression level, and the determined tumor cellularity are all higher than corresponding median levels of the at least one gene product expression, proliferation gene expression and median tumor cellularity obtained from tumor tissue samples from the same first cohort of subjects, while the measured expression level of the at least one gene product associated with cell adhesion, such as the PVR gene product, is below a median expression level of the at least one corresponding gene product associated with cell adhesion, such as the PVR gene product, in the same first cohort of subjects. In other embodiments, the measured expression level of the at least one gene product associated with each of the one or more ADC therapies, the determined proliferation gene expression level, and the determined tumor cellularity may be 10%, 15%, 20%, 25%, 30%, 35%, 45%, 50%, 55%, 60%, 65%, 70%, 75%, 80%, 85%, 90%, 95%, 100%, 125%, 150%, 175%, 200%, 250%, 300% or higher than corresponding median levels of the at least one gene product expression, proliferation gene expression and median tumor cellularity obtained from tumor tissue samples from the same first cohort of subjects. In some other embodiments, the measured expression level of the at least one gene product associated with each of the one or more ADC therapies, the determined proliferation gene expression level, and the determined tumor cellularity may be 1.1 fold, 1.2 fold, 1.25 fold, 1.5 fold, 1.75 fold, 1.9 fold, 2 fold, 3 fold, 4 fold, 5 fold or more higher than corresponding median levels of the at least one gene product expression, proliferation gene expression and median tumor cellularity obtained from tumor tissue samples from the same first cohort of subjects.

In some embodiments, the subject is identified as likely to respond to one or more ADC therapies when the measured expression level of the at least one gene product associated with each of the one or more ADC therapies, the determined proliferation gene expression level, and/or the determined tumor cellularity fall into the highest quartile of the one or more gene products expression levels, proliferation gene expression level and/or tumor cellularity values obtained from tumor tissue samples from the same first cohort of subjects, while the expression level of the at least one gene product associated with cell adherence, such as the PVR gene product, falls into the lowest quartile of expression levels the corresponding at least one gene product associated with cell adherence, such as the PVR gene product, from tumor samples obtained from the same first cohort of subjects. In some embodiments, the subject is identified as likely to respond to the one or more ADC therapies when one or more of the measured expression levels of the at least one gene product, the determined proliferation gene expression level, and/or the determined tumor cellularity fall into the highest top 30%, top 25%, top 20%, top 15%, top 10%, top 5%, top 3% or top 1% of the at least one gene product expression level, proliferation gene expression level and/or tumor cellularity values obtained from tumor tissue samples from the same first cohort of subjects. In some embodiments, the subject is identified as likely to response to the one or more ADC therapies when the expression level of the at least one gene product associated with cell adhesion, such as the PVR gene product, falls into the lowest 30%, lowest 25%, lowest 20%, lowest 15%, lowest 10%, lowest 5%, lowest 3%, or lowest 1% of the expression level of the corresponding at least one gene product associated with cell adhesion, such as the PVR gene product, from tumor tissue samples from the same first cohort of subjects.

Methods of Identifying a Subject Likely to Benefit from Anti-TROP2 Therapy

Sacituzumab govitecan (SG, TRODELVY®), is a Trop-2 ADC that combines a humanized anti-TROP2 monoclonal antibody with the topoisomerase I inhibitor, SN-38, via a cleavable CL2A linker⁵. SG is indicated for unresectable or metastatic triple-negative breast cancer (TNBC) after two or more prior systemic therapies⁵, and locally advanced or metastatic bladder cancer patients who have previously received a platinum-containing chemotherapy and a PD-1 or PD-L1 inhibitor⁶. TROP2 protein expression was evaluated post-hoc in the TNBC study, and while all the objective responses occurred in patients with moderate or strong staining, this represented almost all the study population (88%), providing limited opportunity for stratification⁷. In the IMMU-12-01 basket trial, objective responses were observed in 8 of 9 solid tumor types with 10 or more patients enrolled, with response rates varying from 0% in pancreatic cancer (0/16) to 33.3% in TNBC (36/108)⁸.

Given the significant variability in objective response rates observed across tumor types, there is a need to develop a predictive biomarker of Trop-2 ADC response across solid tumors. Providing such a biomarker would improve the ability to select patients with increased likelihood of benefit from the selected ADC, providing the opportunity to better tailor use within currently approved indications, but also to expand the ADC benefit to additional tumor types.

By leveraging available next generation sequencing (NGS)-based molecular profiling data from an advanced solid tumor cohort (n=23,968), the inventors were able to develop a multivariate biomarker algorithm that predicts the observed objective response rates across tumor types to a selected ADC.

In some aspects, the disclosure is directed to a method of identifying a subject as likely to benefit from an anti-TROP2 based therapy, comprising: (a) measuring the expression level of a TROP2 gene product and one or more gene products associated with proliferation in a tumor tissue sample obtained from the subject; (b) measuring the expression levels of one or more housekeeping genes in the same tumor tissue sample of step (a), wherein the one or more housekeeping genes comprise three genes selected from CIAO1, EIF2B1, HMBS, CTCF, GGNBP2, ITGB7, MYC and SLC4A1AP, and further normalizing the expression levels of the TROP2 gene product and one or more gene products associated with proliferation of step (a) to the three housekeeping genes to obtain normalized expression levels of the TROP2 and one or more genes associated with proliferation; (c) determining proliferation gene expression level by averaging the normalized expression levels of the one or more gene products associated with proliferation; (c) determining tumor cellularity in the same tumor tissue sample of step (a); and (d) identifying the subject as likely to benefit from the anti-TROP2 based therapy when either i) an aggregate biomarker score surpasses a predetermined threshold, wherein the aggregate biomarker score is calculated from the combination of the measured expression level of the TROP2 gene product and at least one of the determined proliferation gene expression level and/or the determined tumor cellularity, or when ii) the measured expression level of the TROP2 gene product is higher than a median TROP2 expression level obtained from tumor tissue samples from a first cohort of subjects, and at least one of the determined proliferation gene expression level and/or the determined tumor cellularity is higher than a median proliferation gene expression level and/or a median tumor cellularity obtained from tumor tissue samples from the same first cohort of subjects.

In some embodiments, the subject is identified as likely to benefit from the anti-TROP2 based therapy, when the measured expression level of the TROP2 gene product, the determined proliferation gene expression level, and the determined tumor cellularity are all higher than corresponding median levels of TROP2 expression, proliferation gene expression and median tumor cellularity obtained from tumor tissue samples from the same first cohort of subjects.

In other embodiments, the measured expression level of the TROP2 gene product, the determined proliferation gene expression level, and the determined tumor cellularity may be 10%, 15%, 20%, 25%, 30%, 35%, 45%, 50%, 55%, 60%, 65%, 70%, 75%, 80%, 85%, 90%, 95%, 100%, 125%, 150%, 175%, 200%, 250%, 300% or higher than corresponding median levels of TROP2 expression, proliferation gene expression and median tumor cellularity obtained from tumor tissue samples from the same first cohort of subjects. In some other embodiments, the measured expression level of the TROP2 gene product, the determined proliferation gene expression level, and the determined tumor cellularity may be 1.1 fold, 1.2 fold, 1.25 fold, 1.5 fold, 1.75 fold, 1.9 fold, 2 fold, 3 fold, 4 fold, 5 fold or more higher than corresponding median levels of TROP2 expression, proliferation gene expression and median tumor cellularity obtained from tumor tissue samples from the same first cohort of subjects.

In some embodiments, the subject is identified as likely to benefit from the anti-TROP2 based therapy when one or more of the measured expression levels of the TROP2 gene product, the determined proliferation gene expression level, and/or the determined tumor cellularity fall into the highest quartile of TROP2 expression level, proliferation gene expression level and/or tumor cellularity values obtained from tumor tissue samples from the same first cohort of subjects. In some embodiments, the first cohort of subjects is a pan-cancer cohort or a matched tumor-type cohort.

In some embodiments, the step of measuring the expression level of one or more gene products associated with proliferation comprises measuring the expression levels of a gene product of one or more genes which are cell cycle regulated and pertain to DNA replication, mitotic processes/phases, spindle assembly, tubulin, mitotic surveillance, cell adhesion, chromosome metabolism, and histone formation. In some embodiments genes with gene products associated with proliferation are one or more genes selected from the group consisting of BIRC5, BRCA1, BRCA2, BUB1, BUB1B, CCNA2, CCNB1, CCNB2, CCNE1, CCNE2, CCNF, CCNG2, CDC2, CDC20, CDC25A, CDC25B, CDC25C, CDC45L, CDC6, CDC7, CDCKN1A p21, CDCKN3, CDK4, CDKN1C, CDKN2A, CDKN2C, CDKN2D p19, CDKN3, CENPA, CENPE, CENPF, CHAF1A, CHK1, CKS1, CKS2, CKS2, DHFR, DHFR, E2-EPF, E2F1, E2F3, E2F5, FEN1, FOXM1, KNSL2, KNSL5, KPNA2, LMNB2, MAD2, MAD2L1, MAPK13, MCM2, MCM3, MCM4, MCM5, MCM6, MK167, MNAT1, MSH2, MYBL2, NASP, NEK2, NPAT, ORC1, PA2G4, PCNA, PES1, PKMYT1, PLK, PRIM1, PTTG1, RAB6KIFL, RACGAP1, RAD51, RAD54L, RAN RAN, RFC4, RPA3, RRM1, RRM2, SLBP, STK12, STK15, STK18, STK6, TOP2A, TROAP, TUBB, TYMS, UBE2C, and UNG. In some embodiments, measuring the expression level of one or more gene products associated with proliferation comprises measuring the expression level of one, two or all three of MYBL2, TOP2A, and/or UBE2C gene products.

In some embodiments, the aggregate biomarker score is determined by the combination of the expression level of TROP2 gene products, the determined proliferation gene expression level, and tumor cellularity. In some embodiments, wherein the predetermined threshold is set to the percentile of ranked aggregate biomarker scores determined from tumor tissue samples of a second cohort of subjects, which percentile corresponds to a percentage of subjects of the second cohort which do not respond to an anti-TROP2 based therapy.

In some embodiments, the second cohort of subjects is a pan-cancer cohort or a matched tumor-type cohort. In some embodiments, the first cohort of subjects and the second cohort of subjects are the same cohort of subjects.

In some embodiments, gene expression levels of TROP2, one or more genes associated with proliferation, and tumor cellularity are log 2 transformed and/or median-centered to 10 prior to step (d). In some embodiments, the tumor cellularity is a molecularly determined tumor cellularity which is calculated based on somatic and germline variant allele frequencies and/or copy number profiles. In some embodiments, one, two or three of the housekeeping genes are selected from CIAO1, EIF2B1, and HMBS.

In some embodiments, the aggregate biomarker score is determined by adding the measured expression level of TROP2 gene product to approximately ⅓ to ⅔ the determined proliferation gene expression level and approximately 4-8 times the determined tumor cellularity.

In some embodiments, the aggregate biomarker score is determined as follows:

$$aggregate biomarker score = [ TROP2 ] + 0.6 * [ Proliferation ] + 6 * [ tumor cellularity ] .$$

In some embodiments, the expression products of TROP2 and one or more genes associated with proliferation are individually selected from a ribonucleic acid (RNA) and a protein. In some embodiments, the gene expression product of at least one of TROP2 and one or more genes associated with proliferation is a protein, and measuring the expression level thereof requires making use of immunohistochemistry techniques. In some embodiments, the gene expression product of at least one of TROP2 and one or more genes associated with proliferation is a RNA, and measuring the expression level thereof requires making use of RNA sequencing techniques.

In some embodiments, the anti-TROP2 based therapy, comprises an anti-TROP2 antibody or fragment thereof. In some embodiments, the anti-TROP2 antibody or fragment thereof is conjugated directly or indirectly to a cytotoxic drug. In some embodiments, the cytotoxic drug is a DNA replication inhibitor selected from the group consisting of an alkylating agent, a DNA polymerase inhibitor, a nitrogen mustard and a topoisomerase inhibitor. In some embodiments, the anti-TROP2 antibody or fragment thereof is fused to a protein which is toxic to a cancer cell. In some embodiments, the cytotoxic drug is a topoisomerase inhibitor. In some embodiments, the anti-TROP2 based therapy is Sacituzumab govitecan.

In some embodiments, subject has or is suspected of having a cancer not approved for labeled use of anti-TROP2 based therapy. In some embodiments, the tumor tissue sample is or is suspected of containing bladder cancer, endometrial cancer, breast cancer, small cell lung cancer, prostate cancer, esophagogastric cancer, non-small cell lung cancer, pancreatic cancer, or colorectal cancer.

In some embodiments, the tumor tissue sample is a formalin-fixed paraffin-embedded (FFPE) tumor tissue sample. In some embodiments, the tumor tissue sample contains at least 20% tumor content. In some embodiments, the method further comprises administering an anti-TROP2 based therapy to the subject identified as likely to benefit from the anti-TROP2 based therapy.

### EXAMPLES

### Example 1

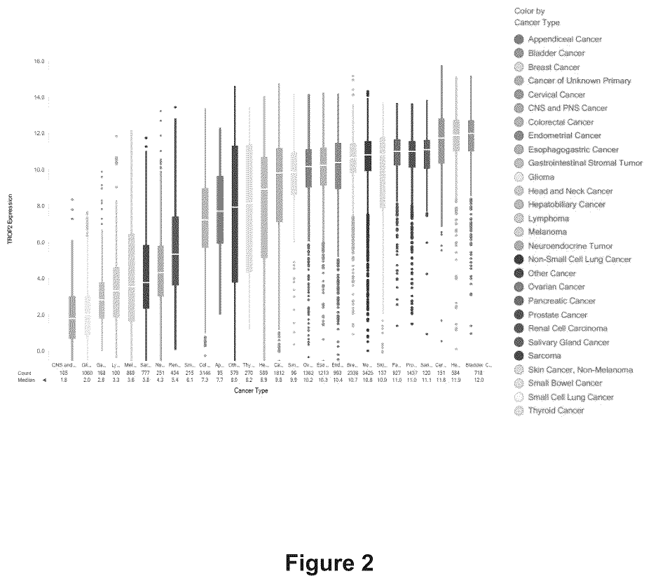

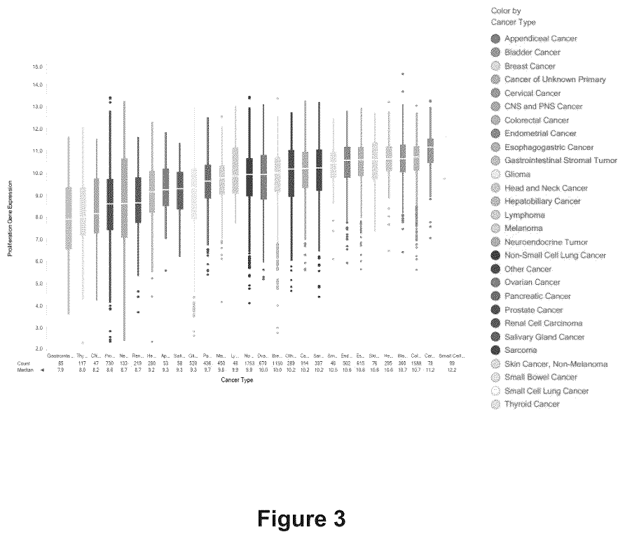

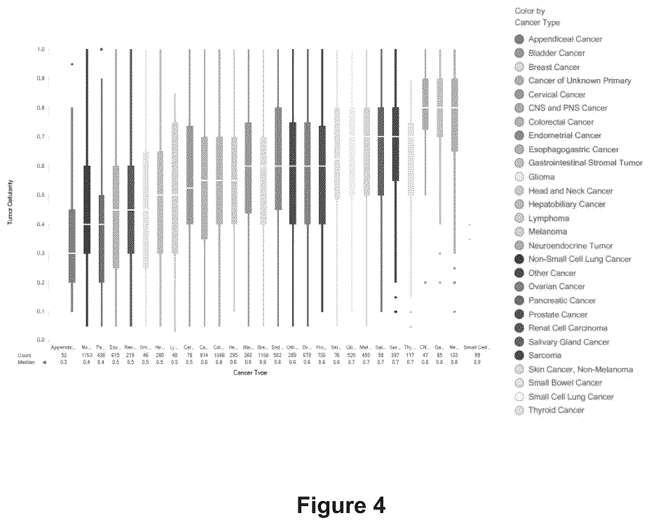

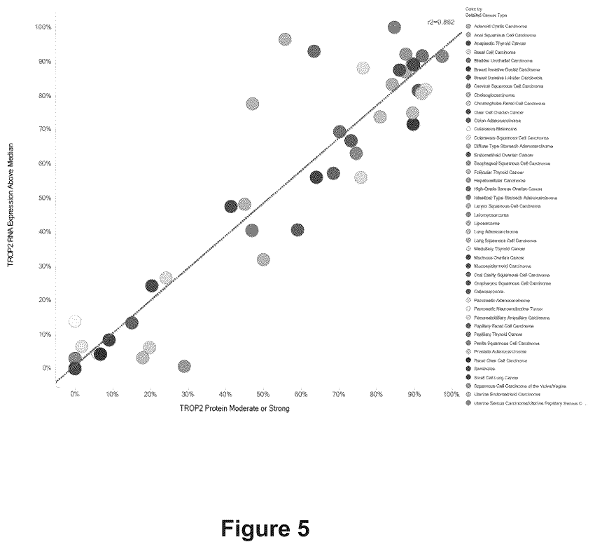

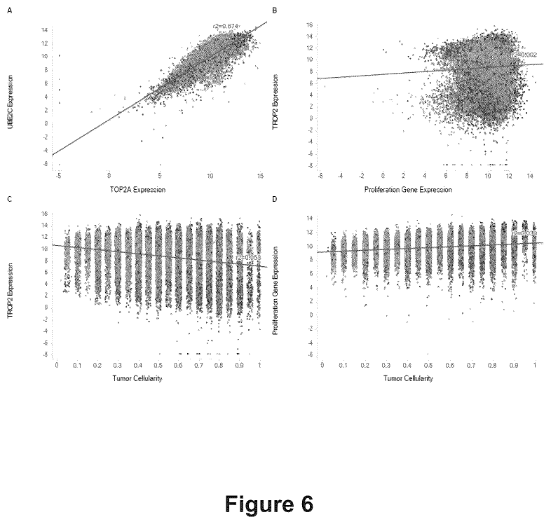

We considered three candidate biomarkers: TROP2 gene expression, cell proliferation gene expression, and molecularly defined tumor cellularity (i.e., the proportion of tumor cells present in the macro-dissected tumor specimen). Across solid tumors, TROP2 showed the highest expression in epithelial tumors such as bladder, head and neck, vaginal and cervical, with the lowest expression observed in non-epithelial tumors such as glioma, GIST, lymphoma, melanoma and sarcoma (FIG. 2). In addition, TROP2 gene expression was highly correlated with protein expression¹⁰ across 45 tumor types (r=0.93, Table 1, FIG. 5). Proliferation gene expression was less variable across tumor types, with small cell lung cancer (SCLC) standing out as the most proliferative tumor type (FIG. 3). Tumor cellularity showed wide variability across and within tumor types, with pancreatic cancer having the lowest median tumor cellularity of 30% and adrenal, CNS, GIST and SCLC having median tumor cellularity at or above 80% (FIG. 4). The three candidate biomarkers were uncorrelated with one another (FIG. 6).

To evaluate the ability of the biomarkers alone and in combination to predict SG response, we randomly divided the 14,410 tumor profiles from the nine tumor types with response data into discovery (n=7,177) and validation cohorts (n=7,233). Based on the weighted mean objective response rate of 14.7%, we fixed the overall positive biomarker rate at 25% for each candidate biomarker, anticipating strong enrichment for response but not perfect concordance. We then evaluated the Pearson correlation of tumor type-specific biomarker rates with objective response rates. The individual biomarkers produced only weak correlations with SG response (FIG. 6), with none reaching statistical significance: TROP2 expression (r=0.38, p=0.23), proliferation gene expression (r=0.15, p=0.76) and tumor cellularity (r=0.35, p=0.43).

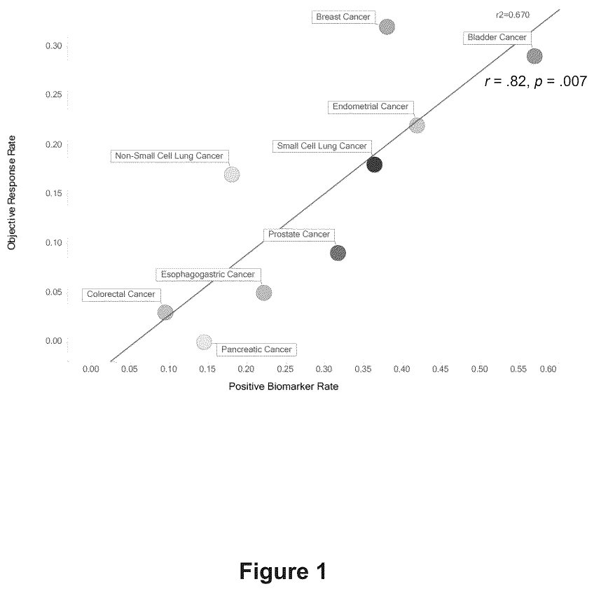

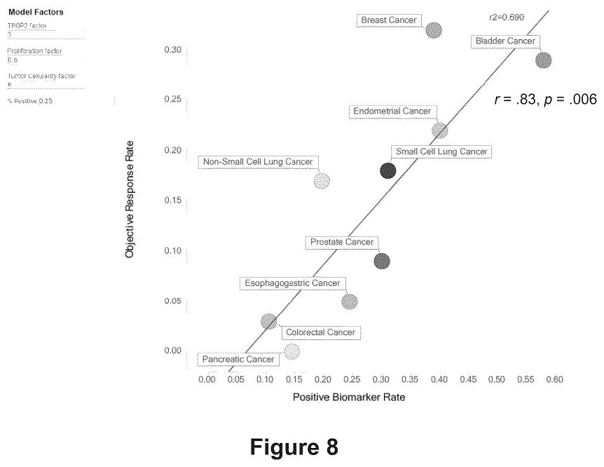

In contrast, an optimized linear equation combining all 3 biomarkers was strongly correlated with response, both when using tumor type-specific biomarker rates derived from the discovery cohort (r=0.83, p=0.006) (FIG. 8) and the independent validation cohort (r=0.82, p=0.007) (FIG. 1).

$$SG biomarker score = [ T R O P 2 ] + 0.6 * [ Proliferation ] + 6 * [ tumor cellularity ]$$

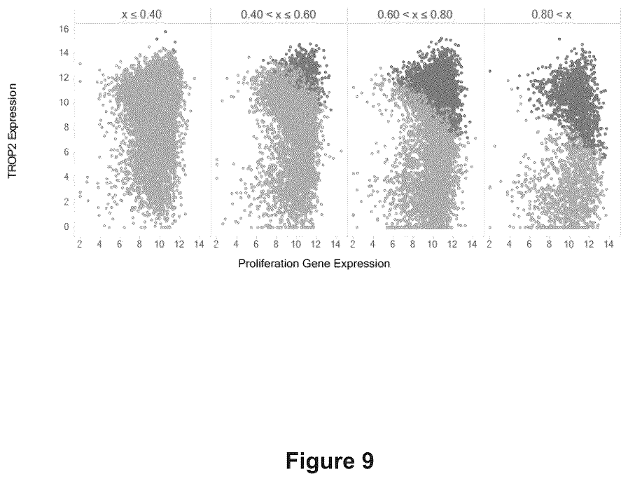

Notably, the biomarker algorithm was far superior to TROP2 expression alone, suggesting that the biomarker factors synergize to impart a clinical response. Considering SG's mechanism of action, a plausible model for response is that (1) higher target expression increases ADC binding, internalization and payload cleavage, (2) higher tumor cellularity increases the proportion of released payload molecules that diffuse into neighboring tumor cells (i.e. ADC bystander effect)¹¹ and (3) higher tumor cell proliferation increases the likelihood of payload molecules blocking DNA replication and causing tumor cell death)¹². The distribution of biomarker factors and positive biomarker calls across the full cohort is depicted in FIG. 9, with the level of TROP2 expression required for a positive biomarker call varying dynamically as a function of tumor cellularity and proliferation gene expression.

Next, we applied the biomarker algorithm to all tumor types represented in the full cohort as outlined in Table 1 below.

TABLE 1
Average SG biomarker scores and biomarker positive rates
by tumor type in the full molecular cohort, grouped by tumor
types with 10 or more patients evaluated in the IMMU-12-01 basket
trial⁸ and sorted by biomarker positive rate.

| | | Average | Biomarker | Objective |
|---|---|---|---|---|
| | | Biomarker | Positive | Response |
| Cancer Type | n | Score | Rate | Rate |
| Bladder Cancer | 718 | 18.5 | 57.4% | 29% |
| Endometrial Cancer | 993 | 16.8 | 40.9% | 22% |
| Breast Cancer | 2,336 | 16.9 | 38.4% | 32% |
| Small Cell Lung Cancer | 215 | 17.5 | 33.5% | 18% |
| Prostate Cancer | 1,437 | 16.4 | 30.8% | 9% |
| Esophagogastric Cancer | 1,213 | 14.0 | 23.2% | 5% |
| Non-Small Cell Lung | 3,425 | 14.2 | 18.8% | 17% |
| Cancer | | | | |
| Pancreatic Cancer | 927 | 12.3 | 14.5% | 0% |
| Colorectal Cancer | 3,146 | 13.6 | 9.9% | 3% |
| Subtotal | 14,410 | 15.0 | 25.0% | 14% |
| Head and Neck Cancer | 584 | 18.1 | 50.5% | |
| Cervical Cancer | 151 | 17.6 | 49.7% | |
| Salivary Gland Cancer | 120 | 17.5 | 40.8% | |
| Skin Cancer, Non- | 137 | 16.5 | 37.2% | |
| Melanoma | | | | |
| Ovarian Cancer | 1,362 | 15.7 | 34.6% | |
| Cancer of Unknown | 1,812 | 14.2 | 23.6% | |
| Primary | | | | |
| Other Cancer | 579 | 13.4 | 21.2% | |
| Small Bowel Cancer | 96 | 13.6 | 17.7% | |
| Thyroid Cancer | 270 | 14.1 | 17.4% | |
| Hepatobiliary Cancer | 589 | 11.5 | 8.0% | |
| Appendiceal Cancer | 95 | 8.0 | 7.4% | |
| Neuroendocrine Tumor | 251 | 12.7 | 6.4% | |
| Lymphoma | 100 | 9.1 | 4.0% | |
| Sarcoma | 777 | 12.3 | 3.6% | |
| Renal Cell Carcinoma | 434 | 9.3 | 2.5% | |
| Melanoma | 868 | 11.5 | 2.1% | |
| Gastrointestinal Stromal | 168 | 11.2 | 0.6% | |
| Tumor | | | | |
| CNS and PNS Cancer | 105 | 10.2 | 0.0% | |
| Glioma | 1,060 | 8.7 | 0.0% | |
| Subtotal | 9,558 | 13.1 | 17.7% | |
| Grand total | 23,968 | 14.2 | 22.1% | |

Among tumor types with responses observed in the basket trial, biomarker positive rates ranged from 9.9% in colorectal cancer to 57.4% in bladder cancer. While the objective response rates in unselected patients were sufficient for FDA approval in TNBC and bladder cancer, selecting patients based on the biomarker algorithm would likely yield improved response rates in the other tumor types. Our analysis identified additional tumor types with high biomarker positive rates, representing a potential opportunity to expand the use of SG further. Cancers of the head and neck, cervix, salivary gland, skin (non-melanoma) and ovary all had positive biomarker rates >30% and rare squamous cell carcinomas of the penis (89%), anus (67%) and vulva (44%) had among the highest biomarker rates. Given that most tumor types had biomarker positive rates >5%, a tumor type-agnostic biomarker approach, similar to that taken for pembrolizumab in tumor mutation burden-high solid tumor patients¹³, could also be considered for SG.

In summary, we uncovered a novel biomarker algorithm capable of predicting SG response across solid tumors. The biomarker may improve the ability to select patients with increased likelihood of benefit from SG, providing the opportunity to better tailor use within currently approved indications, but also to expand SG benefit to additional tumor types. Future studies should further evaluate the biomarker algorithm in patients previously treated with SG and in prospective clinical trials. The biomarker approach of combining target expression with proliferation and tumor cellularity to predict response may be generalizable to ADCs as a class, with the potential to further optimize use and maximize benefit.

### Methods

### Clinical Outcome Dataset

Tumor type-specific objective response rates were collated from the IMMU-12-01 basket trials, selecting the nine tumor types with 10 or more patients enrolled. Given the similar results, the overall breast cancer objective response rate was set as the average of triple negative breast cancer and hormone receptor positive breast cancer.

### Molecular Dataset

Molecular data were collected as part of the Strata Trial (NCT03061305), a large multi-institutional observational study, with StrataNGS (Strata Oncology, Ann Arbor, MI) an analytically validated comprehensive genomic profiling and targeted quantitative transcriptomic profiling next-generation sequencing (NGS) test, as previously described^{14,15}. The molecular dataset included 23,968 formalin-fixed paraffin-embedded tumor biopsy or resection specimens having tumor surface area >2 mm2, consecutively received from Nov. 20, 2019, to Jul. 14, 2022. Other specimen types (e.g., fine needle aspirate, cell block, aspirate) and sizes (tumor surface area <2 mm2) were excluded from analysis. RNA sequencing-based gene expression values were log 2 transformed and median-centered to 10. Proliferation gene expression was calculated as the average of TOP2A and UBE2C. Molecularly-defined tumor cellularity was calculated based on somatic and germline variant allele frequencies and copy number profiles, and then log 2 transformed, as previously described¹⁴.

Gene vs. Protein Expression Analysis

Tumor types from published protein immunohistochemistry data¹⁰ were mapped to the molecular dataset for comparison and the tumor type-specific rates of gene expression above pan-solid tumor median were compared to the moderate or strong staining rates by Pearson's correlation coefficient (FIG. 5, Table 2).

TABLE 2
Comparison of TROP2 gene expression by RNA sequencing (% >
median) to TROP2 protein expression by immunohistochemistry
(% with moderate or strong staining) across 45 tumor types.

| | Gene Expression | | Protein Expression | |
|---|---|---|---|---|
| | | % | | % |
| Detailed Cancer Type | n | Positive | n | Positive |
| Adenoid Cystic Carcinoma | 49 | 78% | 87 | 47% |
| Anal Squamous Cell Carcinoma | 72 | 83% | 69 | 84% |
| Anaplastic Thyroid Cancer | 37 | 24% | 44 | 20% |
| Basal Cell Carcinoma | 15 | 27% | 79 | 24% |
| Bladder Urothelial Carcinoma | 499 | 92% | 587 | 92% |
| Breast Invasive Ductal | 1203 | 72% | 1594 | 90% |
| Carcinoma | | | | |
| Breast Invasive Lobular | 220 | 81% | 309 | 91% |
| Carcinoma | | | | |
| Cervical Squamous Cell | 93 | 91% | 114 | 97% |
| Carcinoma | | | | |
| Cholangiocarcinoma | 241 | 48% | 40 | 45% |
| Chromophobe Renal Cell | 16 | 6% | 153 | 20% |
| Carcinoma | | | | |
| Clear Cell Ovarian Cancer | 59 | 47% | 46 | 41% |
| Colon Adenocarcinoma | 1410 | 13% | 2171 | 15% |
| Cutaneous Melanoma | 308 | 14% | 43 | 0% |
| Cutaneous Squamous Cell | 67 | 88% | 89 | 76% |
| Carcinoma | | | | |
| Diffuse Type Stomach | 69 | 32% | 144 | 50% |
| Adenocarcinoma | | | | |
| Endometrioid Ovarian Cancer | 63 | 67% | 93 | 73% |
| Esophageal Squamous Cell | 122 | 89% | 66 | 89% |
| Carcinoma | | | | |
| Follicular Thyroid Cancer | 31 | 3% | 150 | 18% |
| Hepatocellular Carcinoma | 144 | 1% | 287 | 29% |
| High-Grade Serous Ovarian | 867 | 57% | 514 | 69% |
| Cancer | | | | |
| Intestinal Type Stomach | 37 | 41% | 160 | 47% |
| Adenocarcinoma | | | | |
| Larynx Squamous Cell | 56 | 96% | 88 | 56% |
| Carcinoma | | | | |
| Leiomyosarcoma | 135 | 3% | 76 | 0% |
| Liposarcoma | 18 | 0% | 112 | 0% |
| Lung Adenocarcinoma | 2364 | 75% | 181 | 90% |
| Lung Squamous Cell Carcinoma | 611 | 87% | 72 | 88% |
| Medullary Thyroid Cancer | 31 | 6% | 108 | 2% |
| Mucinous Ovarian Cancer | 25 | 56% | 75 | 64% |
| Mucoepidermoid Carcinoma | 9 | 89% | 242 | 90% |
| Oral Cavity Squamous Cell | 127 | 93% | 115 | 63% |
| Carcinoma | | | | |
| Oropharynx Squamous Cell | 184 | 88% | 57 | 86% |
| Carcinoma | | | | |
| Osteosarcoma | 17 | 0% | 34 | 0% |
| Pancreatic Adenocarcinoma | 874 | 82% | 383 | 93% |
| Pancreatic Neuroendocrine | 44 | 5% | 83 | 6% |
| Tumor | | | | |
| Pancreatobiliary Ampullary | 41 | 56% | 66 | 76% |
| Carcinoma | | | | |
| Papillary Renal Cell Carcinoma | 32 | 41% | 339 | 59% |
| Papillary Thyroid Cancer | 124 | 69% | 374 | 70% |
| Penile Squamous Cell | 9 | 100% | 78 | 85% |
| Carcinoma | | | | |
| Prostate Adenocarcinoma | 1366 | 81% | 497 | 92% |
| Renal Clear Cell Carcinoma | 260 | 4% | 1180 | 7% |
| Seminoma | 3 | 0% | 592 | 0% |
| Small Cell Lung Cancer | 215 | 8% | 11 | 9% |
| Squamous Cell Carcinoma of | 64 | 92% | 155 | 88% |
| the Vulva | | | | |
| Uterine Endometrioid | 397 | 74% | 203 | 81% |
| Carcinoma | | | | |
| Uterine Serous Carcinoma | 233 | 63% | 55 | 75% |
| Grand total | 12861 | 62% | 12015 | 69% |

### Predictive Biomarker Analysis

The molecular dataset was randomly divided into discovery and validation cohorts for the purpose of multivariate biomarker optimization (discovery) and evaluation (validation). For each quantitative biomarker evaluated (TROP2, proliferation gene expression, tumor cellularity, SG biomarker score), biomarker thresholds were set such that the top 25% of samples in the nine tumor types with response data were biomarker positive and the bottom 75% were biomarker negative. The relationship between tumor type-specific positive biomarker rates and objective response rate was assessed with Pearson's correlation coefficient. The SG biomarker score coefficients were optimized in the discovery cohort by fixing the TROP2 coefficient to 1.0 and the biomarker positive rate to 25% and then varying the proliferation gene expression and tumor cellularity coefficients by 0.1 increments to maximize the Pearson correlation. The SG biomarker score was then evaluated using the validation cohort.

### Example 2

In an effort to further enhance the predictive power of the ADC Treatment Response Score as a pan-cancer predictor of response to ADCs therapies as a group, consideration was given to additional molecular variables. Data used in this example included 25 published ADC objective response rates (ORRs), along with over 15,000 high-quality v4 RNA libraries obtained from the Strata Trial (NCT03061305).

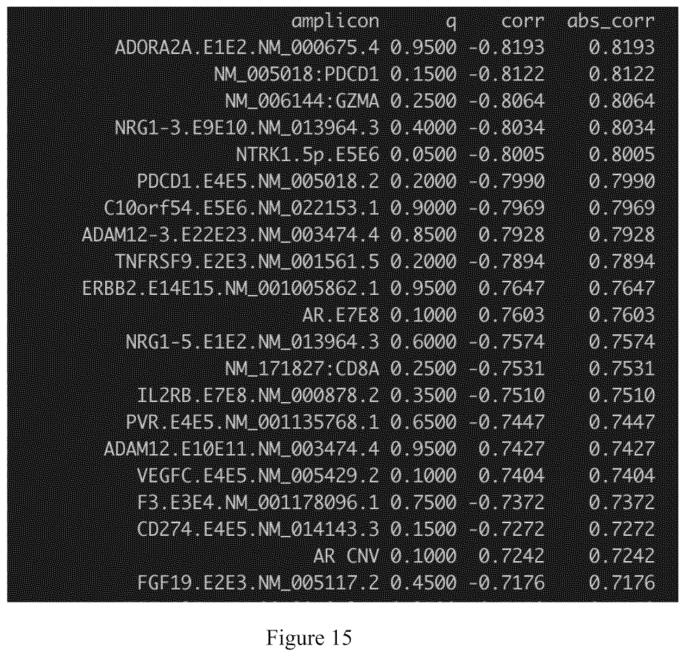

Initially, molecular candidates were identified by analyzing and ranking the absolute correlation between available distinct gene product expression levels with published ADC objective response rates. The top twenty ranked gene expression products and Copy Number Variant (CNV) genes were selected for further investigation, as shown in FIG. 15.

The selected top 20 gene expression products and CNV were trained on all non-TROP2 ADC objective response rates (n=16), by adding each individually to the previously identified variables that included expression level of gene products targeted by a corresponding ADC (ADC target), tumor cellularity (TC) and/or proliferation gene expression (proliferation). Leave-one out cross validation (LOOCV) was performed on the training set, while holding out TACSTD2 ADC ORR data points. All variables were Z score normalized prior to subjecting to cross validation. Parameters of p<0.05 and combined correlation coefficient (CCC) >0.8 were used to screen the aggregate candidate biomarkers for consideration for validation on the TROP2 dataset. During the validation, parameters of q <0.05 and CCC >0.8 were used as cut-offs for discerning validated aggregate biomarkers. Surprisingly, when combined with ADC Target, Proliferation, and optionally TC, PVR gene expression was the only top ranked gene expression variable that provided a validated, robust ADC Treatment Response Score despite multiple validation attempts. Thus, the inventors have shown that the claimed ADC Treatment Response Score is superior to similar predictors which comprise gene expression parameters, that while appearing to be highly correlated with observed response rate in the training set, fail validation.

### Example 3

To facilitate identification of additional model feature candidates that would further improve the sensitivity and specificity of the model, an analysis of whole transcriptome RNA read count data (n=33,070) publicly available through ARCHS⁴ was performed.¹⁷ First, the raw expression read count data was normalized using the DESeq2 method, which takes into account RNA alternative splicing.¹⁸ Next, cancer tissue cohorts for each tissue type were split into two groups via pan-tumor quartiles and the resulting tissue-specific population fractions were then compared to corresponding tumor-specific objective response rates across various ADC targets via Pearson correlation. The resulting list was rank ordered by anti-correlation to identify candidates that might counterbalance the primary model factors, ADC target expression and proliferation, which are both positively correlated with response rates. The candidate list was filtered to exclude genes whose normal function exhibited tissue-specific variance that could confound response correlation analysis. Thus, only genes where each tissue type's fraction above the pan-tissue median was between 30% and 70% were considered for subsequent analysis. Finally, genes whose expression in the normal tissue dataset was anticorrelated with ADC response rates (Pearson correlation <−0.3) were eliminated from further consideration since their correlations in the cancer dataset may not be disease related.

The remaining 500 most anti-correlated genes were then examined via gene set enrichment analysis to identify any patterns of common molecular function, biological function, or cellular component. These genes were found to be highly enriched for a cellular component cluster involving cell adhesion, with 27 of the 500 genes involved in two or more related cellular components that include adherens junction, anchoring junction, cell-substrate adherens junction, cell-substrate junction, and focal adhesion. These 27 genes and the related cellular components which they impact are found below in Table 3.

TABLE 3

| | | | Cell- | | |
|---|---|---|---|---|---|
| | | | Substrate | Cell- | |
| | Adherens | Anchoring | Adherens | Substrate | Focal |
| Gene | Junction | Junction | Junction | Junction | Adhesion |
| ATP2A2 | X | X | | | |
| BAIAP2 | X | X | | | |
| CD151 | X | X | X | X | X |
| CHP1 | X | X | X | X | X |
| CYFIP1 | X | X | X | X | X |
| CYTH3 | X | X | | | |
| DAG1 | X | X | X | X | X |
| DSC2 | X | X | | | |
| GIT1 | X | X | X | X | X |
| HSP90B1 | X | X | X | X | X |
| HSPA5 | X | X | X | X | X |
| LIMK1 | X | X | X | X | X |
| MAPK1 | X | X | X | X | X |
| PACSIN2 | X | X | X | X | X |
| PDIA3 | X | X | X | X | X |
| PVR | X | X | X | X | X |
| REXO2 | X | X | X | X | X |
| RPL22 | X | X | X | X | X |
| RPLP1 | X | X | X | X | X |
| RPLP2 | X | X | X | X | X |
| RPS11 | X | X | X | X | X |
| RPS16 | X | X | X | X | X |
| RPS5 | X | X | X | X | X |
| SDCBP | X | X | X | X | X |
| SNAP23 | X | X | X | X | X |
| SNTB1 | X | X | X | X | X |
| SRP68 | X | X | X | X | X |

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### REFERENCES

1 Fu, Z., Li, S., Han, S., Shi, C. & Zhang, Y. Antibody drug conjugate: the “biological missile” for targeted cancer therapy. Signal Transduction and Targeted Therapy 7, 93 (2022). https://doi.org:10.1038/s41392-022-00947-7
2 Verma, S. et al. Trastuzumab Emtansine for HER2-Positive Advanced Breast Cancer. New England Journal of Medicine 367, 1783-1791 (2012). https://doi.org:10.1056/NEJMoa1209124
3 Modi, S. et al. Trastuzumab Deruxtecan in Previously Treated HER2-Positive Breast Cancer. New England Journal of Medicine 382, 610-621 (2019). https://doi.org:10.1056/NEJMoa1914510
4 Ab, O. et al. IMGN853, a Folate Receptor-α (FRα)-Targeting Antibody-Drug Conjugate, Exhibits Potent Targeted Antitumor Activity against FRα-Expressing Tumors. Molecular Cancer Therapeutics 14, 1605-1613 (2015). https://doi.org:10.1158/1535-7163.Mct-14-1095
5 Bardia, A. et al. Sacituzumab Govitecan-hziy in Refractory Metastatic Triple-Negative Breast Cancer. New England Journal of Medicine 380, 741-751 (2019). https://doi.org:10.1056/NEJMoa1814213
6 Tagawa, S. T. et al. TROPHY-U-01: A Phase II Open-Label Study of Sacituzumab Govitecan in Patients With Metastatic Urothelial Carcinoma Progressing After Platinum-Based Chemotherapy and Checkpoint Inhibitors. Journal of Clinical Oncology 39, 2474-2485 (2021). https://doi.org:10.1200/jco.20.03489
7 Bardia, A. et al. Efficacy and Safety of Anti-Trop-2 Antibody Drug Conjugate Sacituzumab Govitecan (IMMU-132) in Heavily Pretreated Patients With Metastatic Triple-Negative Breast Cancer. Journal of Clinical Oncology 35, 2141-2148 (2017). https://doi.org:10.1200/jco.2016.70.8297
8 Bardia, A. et al. Sacituzumab govitecan, a Trop-2-directed antibody-drug conjugate, for patients with epithelial cancer: final safety and efficacy results from the phase I/II IMMU-132-01 basket trial. Annals of Oncology 32, 746-756 (2021). https://doi.org:https://doi.org/10.1016/j.annonc.2021.03.005
9 Lee, J. S. & Ruppin, E. Multiomics Prediction of Response Rates to Therapies to Inhibit Programmed Cell Death 1 and Programmed Cell Death 1 Ligand 1. JAMA Oncology 5, 1614-1618 (2019). https://doi.org:10.1001/jamaoncol.2019.2311
10 Dum, D. et al. Trophoblast Cell Surface Antigen 2 Expression in Human Tumors: A Tissue Microarray Study on 18,563 Tumors. Pathobiology 89, 245-258 (2022). https://doi.org:10.1159/000522206
11 Singh, A. P., Sharma, S. & Shah, D. K. Quantitative characterization of in vitro bystander effect of antibody-drug conjugates. Journal of Pharmacokinetics and Pharmacodynamics 43, 567-582 (2016). https://doi.org:10.1007/s10928-016-9495-8
12 Pommier, Y. Topoisomerase I inhibitors: camptothecins and beyond. Nature Reviews Cancer 6, 789-802 (2006). https://doi.org:10.1038/nrc1977
13 Marabelle, A. et al. Association of tumour mutational burden with outcomes in patients with advanced solid tumours treated with pembrolizumab: prospective biomarker analysis of the multicohort, open-label, phase 2 KEYNOTE-158 study. The Lancet Oncology 21, 1353-1365 (2020). https://doi.org:https://doi.org/10.1016/S1470-2045(20)30445-9
14 Tomlins, S. A. et al. Development and Validation of StrataNGS, a Multiplex PCR, Semiconductor Sequencing-Based Comprehensive Genomic Profiling Test. The Journal of Molecular Diagnostics 23, 1515-1533 (2021). https://doi.org:https://doi.org/10.1016/i.imoldx.2021.08.005
15 Khazanov, S., Lamb, et al. Prediction of pan-solid tumor pembrolizumab benet by integrating tumor mutation and gene expression proling (2022).
16 Coats, Steven, et al. “Antibody—Drug Conjugates: Future Directions in Clinical and Translational Strategies to Improve the Therapeutic IndexAdvances in Antibody—Drug Conjugate Clinical Development.” Clinical Cancer Research 25.18 (2019): 5441-5448, https://aacrjournals.org/clincancerres/article/25/18/544/81676/Antibody-Drug-Conjugates-Future-Directions-in
17 Icahn School of Medicine at Mount Sinai, Metadata Search, The Ma'ayan Lab. https://maavanlab.cloud/archs4/data.html
18 Love, M. I., Huber, W. & Anders, S. Moderated estimation of fold change and dispersion for RNA-seq data with DESeq2. Genome Biol 15, 550 (2014). https://doi.org/10.1186/s13059-014-0550-8
19 Loriot, Yohann, et al. “Efficacy of sacituzumab govitecan (SG) in locally advanced (LA) or metastatic urothelial cancer (rnUC) by trophoblast cell surface antigen 2 (Trop-2) expression.” (2023): 4579-4579.
20 Bardia, Aditya, et al. “Trop-2 mRNA expression and association with clinical outcomes with sacituzumab govitecan (SG) in patients with HR+/HER2-metastatic breast cancer (mBC): Biomarker results from the phase 3 TROPiCS-02 study,” (2023): 1082-1082.

1.-146. (canceled)

147. A method for identifying a subject as likely to respond to one or more antibody-drug conjugate (ADC) therapies, the method comprising the steps of:
(A) measuring the expression level of at least one gene product associated with each of the one or more ADC therapies from a biological tissue sample obtained from the subject;
(B) measuring in the same biological tissue sample of step (A), the expression level of one or both of:
(i) at least one gene product associated with cell adhesion, and
(ii) one or more gene products associated with proliferation,
wherein if the expression level of more than one gene product associated with proliferation is measured, calculating therefrom an average of all expression levels of the measured gene products associated with proliferation, thereby obtaining a proliferation gene expression level;
(C) optionally, determining tumor cellularity in the same tumor tissue sample of steps (A) and (B);
(D)(1) identifying a subject likely to respond to the one or more ADC therapies when one or more of a calculated ADC Treatment Response Score (ADC TRS) surpass one or more corresponding predetermined thresholds associated with a positive response to the one or more ADC therapies, wherein each of the one or more ADC TRS is determined from:
(i) the measured expression level of the at least one gene product associated with a corresponding ADC therapy,
and at least two of:
(ii) the measured expression level of at least one gene product associated with cell adhesion,
(iii) the determined proliferation gene expression level, and
(iv) the determined tumor cellularity;
optionally, wherein the ADC TRS is determined by at least the combination of the expression level of the at least one gene product associated with a corresponding ADC therapy, the expression level of at least one gene product associated with cell adhesion, and the determined proliferation gene expression level; and/or
wherein both the determined proliferation gene expression level and the expression level of the at least one gene product associated with a corresponding ADC therapy are positively associated with a likelihood that a patient will respond to the corresponding ADC therapy, and the expression level of at least one gene product associated with cell adhesion is negatively associated with a likelihood that a patient will respond to the same corresponding ADC therapy; and/or
wherein the ADC TRS is determined by at least the combination of the expression level of the at least one gene product associated with a corresponding ADC therapy, the measured expression level of at least one gene product associated with cell adhesion, and tumor cellularity,
optionally wherein both the tumor cellularity and the expression level of the at least one gene product associated with a corresponding ADC therapy are positively associated with a likelihood that a patient will respond to the corresponding ADC therapy, and the expression level of at least one gene product associated with cell adhesion is negatively associated with a likelihood that a patient will respond to the same corresponding ADC therapy; and/or
wherein the ADC TRS is determined by at least the combination of the expression level of the at least one gene product associated with a corresponding ADC therapy, the expression level of at least one gene product associated with cell adhesion, the determined proliferation gene expression level, and tumor cellularity;
optionally, wherein each of the determined proliferation gene expression level, the tumor cellularity, and the expression level of the at least one gene product associated with a corresponding ADC therapy are positively associated with a likelihood that a patient will respond to the corresponding ADC therapy, and the expression level of at least one gene product associated with cell adhesion is negatively associated with a likelihood that a patient will respond to the same corresponding ADC therapy.

148. The method of claim 147, wherein the at least one gene product associated with each of the one or more ADC therapies comprise RNA transcripts individually selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5; and/or
wherein the step of measuring the expression level of one or more gene products associated with proliferation comprises measuring the expression levels of a gene product of one or more genes selected from the group consisting of BIRC5, BRCA1, BRCA2, BUB1, BUB1B, CCNA2, CCNB1, CCNB2, CCNE1, CCNE2, CCNF, CCNG2, CDC2, CDC20, CDC25A, CDC25B, CDC25C, CDC45L, CDC6, CDC7, CDCKN1A p21, CDCKN3, CDK4, CDKN1C, CDKN2A, CDKN2C, CDKN2D p19, CDKN3, CENPA, CENPE, CENPF, CHAF1A, CHK1, CKS1, CKS2, CKS2, DHFR, DHFR, E2-EPF, E2F1, E2F3, E2F5, FEN1, FOXM1, KNSL2, KNSL5, KPNA2, LMNB2, MAD2, MAD2L1, MAPK13, MCM2, MCM3, MCM4, MCM5, MCM6, MK167, MNAT1, MSH2, MYBL2, NASP, NEK2, NPAT, ORC1, PA2G4, PCNA, PES1, PKMYT1, PLK, PRIM1, PTTG1, RAB6KIFL, RACGAP1, RAD51, RAD54L, RAN RAN, RFC4, RPA3, RRM1, RRM2, SLBP, STK12, STK15, STK18, STK6, TOP2A, TROAP, TUBB, TYMS, UBE2C, and UNG; and/or
wherein the step of measuring the expression level of one or more gene products associated with proliferation comprises measuring the expression level of one, two or all three of MYBL2, TOP2A, and/or UBE2C gene products; and/or
wherein each of the gene products associated with cell adhesion impact at least two of an adherens junction, an anchoring junction, a cell-substrate adherens junction, a cell-substrate junction or a focal adhesion of a cell; and/or
wherein the step of measuring the expression level of at least one gene product associated with cell adhesion comprises measuring the expression level of a gene product of at least one gene selected from the group consisting of ATP2A2, BAIAP2, CD151, CHP1, CYFIP1, CYTH3, DAG1, DSC2, GIT1, HSP90B1, HSPA5, LIMK1, MAPK1, PACSIN2, PDIA3, PVR, REXO2, RPL22, RPLP1, RPLP2, RPS11, RPS16, RPS5, SDCBP SNAP23, SNTB1, and SRP68, optionally,
wherein the step of measuring the expression level of at least one gene product associated with cell adhesion comprises measuring the expression of a PVR gene product, optionally,
wherein the step of measuring the expression level of at least one gene product associated with cell adhesion consists of measuring the expression level of a single gene product associated with cell adhesion, optionally,
wherein the single gene product is a PVR gene product or is not a PVR gene product; and/or
wherein measuring the expression level of at least one gene product associated with cell adhesion comprises measuring the expression level of two or more gene products associated with cell adhesion, and subsequently determining a cell adhesion gene product expression level by averaging the expression of two or more gene products associated with cell adhesion; and/or
wherein the expression level of the at least one gene product associated with a corresponding ADC therapy is weighted by a factor of approximately 0.3 to 0.65, the expression level of at least one gene product associated with cell adhesion is weighted by a factor of approximately −0.8 to −1, and the determined proliferation gene expression level is weighted by a factor of approximately 0.2 to 0.4; and/or
wherein the expression level of the at least one gene product associated with a corresponding ADC therapy is weighted by a factor of approximately 0.3, the expression level of at least one gene product associated with cell adhesion is weighted by a factor of approximately −0.9, and the tumor cellularity is weighted by a factor of approximately 0.8; and/or
wherein the expression level of the at least one gene product associated with a corresponding ADC therapy is weighted by a factor of approximately 0.45, the expression level of at least one gene product associated with cell adhesion is weighted by a factor of approximately −1, the determined proliferation gene expression level is weighted by a factor of approximately 0.55, and the tumor cellularity is weighted by a factor of approximately 0.07; and/or
wherein each of the one or more ADC TRS is determined by further taking into account a bias variable, wherein the bias variable is a static offset tuned to yield biomarker frequencies that match published objective response rates in clinical trials, optionally,
wherein the bias variable is weighted by a factor of approximately −0.25.

149. The method of claim 147, wherein the predetermined threshold is set to a percentile of ranked ADC Treatment Response Scores determined from tumor tissue samples of a first cohort of subjects utilizing at least the expression level of the at least one gene product associated with a first corresponding ADC therapy, wherein each percentile corresponds to a percentage greater than a percentage of subjects of the first cohort which do not respond to the same first corresponding ADC therapy; or
wherein the predetermined thresholds are set to percentiles of ranked ADC Treatment Response Scores determined from tumor tissue samples of a first and a second cohort of subjects utilizing at least the expression level of at least a first and a second gene product associated with a first and second corresponding ADC therapy, wherein each percentile corresponds to a percentage greater than a percentage of subjects of the first and second cohort which do not respond to the at least first and second corresponding ADC therapies, optionally, wherein the first and/or second cohort of subjects is a pan-cancer cohort or a matched tumor-type cohort, optionally, wherein the first cohort of subjects and the second cohort of subjects are the same cohort of subjects; and/or
wherein the predetermined threshold is zero, and an ADC TRS which indicates the subject is likely to respond to an ADC therapy is an ADC TRS with a value greater than zero; and/or
wherein the expression level of the at least one gene product associated with each one or more ADC therapies, the expression level of at least one gene product associated with cell adhesion, and the tumor cellularity are log 2 transformed and/or Z score normalized prior to step (D)(1), and the expression level of the one or more gene products associated with proliferation are log 2 transformed and/or Z score normalized prior to the step of averaging expression levels of gene products associated with proliferation to obtain the proliferation gene expression level in step (B); and/or
wherein the tumor cellularity is a molecularly determined tumor cellularity which is calculated based on somatic and germline variant allele frequencies and/or copy number profiles; and/or
wherein the method further comprises measuring the expression level of at least one housekeeping gene selected from CIAO1, EIF2B1, and HMBS in the tumor tissue sample, and normalizing the expression levels of the at least one gene product associated with the one or more ADC therapies, the at least one gene product associated with cell adhesion and the one or more gene products associated with proliferation to the at least one housekeeping gene expression level to obtain normalized expression levels of the at least one gene product associated with each of the one or more ADC therapies, the at least one gene product associated with cell adhesion, and the one or more gene products associated with proliferation; and/or
wherein the expression product of the at least one gene associated with each of the one or more ADC therapies, cell adhesion, and one or more genes associated with proliferation are individually selected from a ribonucleic acid (RNA) and a protein; and/or
wherein the gene expression product of the at least one gene associated with each of the one or more ADC therapies, cell adhesion and the one or more genes associated with proliferation are proteins, and measuring the expression level thereof requires making use of immunohistochemistry techniques; and/or
wherein the gene expression product of the at least one gene associated with each of the one or more ADC therapies, cell adhesion, and the one or more genes associated with proliferation are RNA, and measuring the expression level thereof requires making use of RNA sequencing techniques or qPCR; and/or
wherein each of the one or more ADC therapies comprise a monoclonal antibody, at least one functional fragment thereof or a bispecific antibody which targets at least one epitope of at least one antigen selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5, optionally, wherein the antibody, the at least one functional fragment thereof, or the bispecific antibody is either fused to a protein which is toxic to a cancer cell, or is conjugated directly or indirectly to a cytotoxic drug, optionally, wherein the cytotoxic drug is a DNA replication inhibitor selected from the group consisting of an alkylating agent, a DNA polymerase inhibitor, a nitrogen mustard and a topoisomerase inhibitor, optionally, wherein the cytotoxic drug is a topoisomerase inhibitor; and/or
wherein the antibody comprises a bispecific antibody capable of targeting two epitopes of the same antigen or an epitope of two distinct antigens, wherein the same antigen or the two antigens are selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5.

150. The method of claim 147, wherein the subject has or is suspected of having a cancer not approved for labeled use of the one or more ADC therapies; and/or
wherein the tumor tissue sample is or is suspected of containing bladder cancer, salivary gland cancer, endometrial cancer, ovarian cancer, cervical cancer, head and neck cancer, non-melanoma skin cancer, thyroid cancer, cancer of unknown primary, cancer of the central or peripheral nervous system, neuroendocrine tumor, melanoma, esophagogastric cancer, small bowel cancer, sarcoma, hepatobiliary cancer, pancreatic cancer, gastrointestinal stromal tumor, renal cell carcinoma, glioma, appendiceal cancer breast cancer, small cell lung cancer, prostate cancer, esophagogastric cancer, non-small cell lung cancer, pancreatic cancer, lymphoma or colorectal cancer, optionally,
wherein the tumor tissue sample is a formalin-fixed paraffin-embedded (FFPE) tumor tissue sample, optionally,
wherein the tumor tissue sample contains at least 20% tumor content.

151. The method of claim 147, further comprising the step (E) administering the at least one of the one or more ADC therapies to a subject identified in step (D)(1) as likely to respond to the one or more ADC therapies; and/or
wherein each of the one or more ADC TRS is determined without taking into account tumor cellularity.

152. A method for selecting one or more antibody-drug conjugate (ADC) therapies among two or more ADC therapies identified as most beneficial to treat a cancer in a subject, the method comprising the steps of:
(A) measuring the expression level of at least one gene product associated with each of the or more ADC therapies from a biological tissue sample obtained from the subject;
(B) measuring in the same biological tissue sample of step (A), the expression level of one or both of:
(i) at least one gene product associated with cell adhesion, and
(ii) one or more gene products associated with proliferation,
wherein if the expression level of more than one gene product associated with proliferation is measured, calculating therefrom an average of all expression levels of the measured gene products associated with proliferation, thereby obtaining a proliferation gene expression level;
(C) optionally, determining tumor cellularity in the same tumor tissue sample of steps (A) and (B);
(D)(1) calculating an ADC Treatment Response Score (ADC TRS) for each of the two or more ADC therapies, and determining each of the two or more ADC TRS surpass a predetermined threshold associated with beneficial patient treatment outcome, wherein each of the one or more ADC TRS is determined from:
(i) the measured expression level of the at least one gene product associated with a corresponding ADC therapy,
and at least two of:
(ii) the measured expression level of at least one gene product associated with cell adhesion,
(iii) the determined proliferation gene expression level, and
(iv) the determined tumor cellularity;
(D)(2) wherein if ADC TRS associated with two or more ADC therapies surpass a predetermined threshold associated with a beneficial patient treatment outcome, ranking the at least two ADC TRS by the value by which each ADC Treatment Response Score exceeds the predetermined threshold, and selecting the highest ranked ADC therapy for administration to a subject; optionally,
further comprising step (E) of administering to the subject the selected highest ranked ADC therapy, optionally,
wherein (E) further comprises administering to the subject at least one other lower ranked ADC exceeding the predetermined threshold in combination with the highest ranked ADC therapy, or
wherein step (E) does not include administering another ADC therapy in combination with the highest ranked ADC therapy.

153. A method for treating a cancer in a subject determined likely to respond to one or more antibody-drug conjugate (ADC) therapies, comprising:
(a) measuring, in a tumor tissue sample obtained from the subject, the expression level of:
i) at least one gene product associated with each corresponding one or more ADC therapies, and at least one of the expression levels of:
ii) at least one gene product associated with cell adhesion, and
iii) one or more gene products associated with proliferation;
(b) measuring the expression levels of one or more housekeeping genes in the same tumor tissue sample of step (a) and further normalizing the expression level of the at least one gene product associated with each of the one or more ADC therapies, the at least one gene product associated with cell adhesion, and one or more gene products associated with proliferation of step (a) against the expression levels of the one or more housekeeping genes to obtain normalized expression levels of the at least one gene product associated with each corresponding one or more ADC therapies, a gene product associated with cell adhesion, and one or more genes associated with proliferation;
(c) if gene products of one or more genes associated with proliferation are measured and normalized, determining proliferation gene expression level by averaging the normalized expression levels of the one or more gene products associated with proliferation;
(d) optionally, determining tumor cellularity in the same tumor tissue sample of steps (a) and (b);
(e)(1) identifying a subject likely to benefit from the one or more ADC therapies when one or more of a calculated ADC Treatment Response Score (ADC TRS) surpass one or more corresponding predetermined thresholds, wherein each of the one or more ADC TRS is determined from:
(i) the measured expression level of the at least one gene product associated with a corresponding ADC therapy,
and at least two of:
(ii) the measured level of the at least one gene product associated with cell adhesion,
(iii) the determined proliferation gene expression level, and
(iv) the determined tumor cellularity; and
(f) administering an effective amount of the one or more ADC therapies to a subject identified as likely to benefit from the one or more ADC therapies; optionally, wherein the ADC TRS is determined by at least the combination of the expression level of the at least one gene product associated with a corresponding ADC therapy, the measured expression level of the at least one gene product associated with cell adhesion, and the determined proliferation gene expression level; and/or
wherein both the determined proliferation gene expression level and the expression level of the at least one gene product associated with a corresponding ADC therapy are positively associated with a likelihood that a patient will respond to the corresponding ADC therapy, and the measured expression level of the at least one gene product associated with cell adhesion is negatively associated with a likelihood that a patient will respond to the same corresponding ADC therapy; and/or
wherein the ADC TRS is determined by at least the combination of the expression level of the at least one gene product associated with a corresponding ADC therapy, the measured expression level of the at least one gene product associated with cell adhesion, and tumor cellularity; and/or
wherein both the tumor cellularity and the expression level of the at least one gene product associated with a corresponding ADC therapy are positively associated with a likelihood that a patient will respond to the corresponding ADC therapy, and the measured expression level of the at least one gene product associated with cell adhesion is negatively associated with a likelihood that a patient will respond to the same corresponding ADC therapy; and/or
wherein the ADC TRS is determined by at least the combination of the expression level of the at least one gene product associated with a corresponding ADC therapy, the measured expression level of at least one gene product associated with cell adhesion, the determined proliferation gene expression level, and tumor cellularity; and/or
wherein each of the determined proliferation gene expression level, the tumor cellularity, and the expression level of the at least one gene product associated with a corresponding ADC therapy are positively associated with a likelihood that a patient will respond to the corresponding ADC therapy, and the measured expression level of at least one gene product associated with cell adhesion is negatively associated with a likelihood that a patient will respond to the same corresponding ADC therapy; and/or
wherein the one or more housekeeping genes comprise three genes selected from CIAO1, EIF2B1, HMBS, CTCF, GGNBP2, ITGB7, MYC and SLC4A1AP.

154. The method of claim 153, wherein the at least one gene product associated with each of the one or more ADC therapies comprise RNA transcripts individually selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5; and/or
wherein the step of measuring the expression level of one or more gene products associated with proliferation comprises measuring the expression levels of a gene product of one or more genes selected from the group consisting of BIRC5, BRCA1, BRCA2, BUB1, BUB1B, CCNA2, CCNB1, CCNB2, CCNE1, CCNE2, CCNF, CCNG2, CDC2, CDC20, CDC25A, CDC25B, CDC25C, CDC45L, CDC6, CDC7, CDCKN1A p21, CDCKN3, CDK4, CDKN1C, CDKN2A, CDKN2C, CDKN2D p19, CDKN3, CENPA, CENPE, CENPF, CHAF1A, CHK1, CKS1, CKS2, CKS2, DHFR, DHFR, E2-EPF, E2F1, E2F3, E2F5, FEN1, FOXM1, KNSL2, KNSL5, KPNA2, LMNB2, MAD2, MAD2L1, MAPK13, MCM2, MCM3, MCM4, MCM5, MCM6, MK167, MNAT1, MSH2, MYBL2, NASP, NEK2, NPAT, ORC1, PA2G4, PCNA, PES1, PKMYT1, PLK, PRIM1, PTTG1, RAB6KIFL, RACGAP1, RAD51, RAD54L, RAN RAN, RFC4, RPA3, RRM1, RRM2, SLBP, STK12, STK15, STK18, STK6, TOP2A, TROAP, TUBB, TYMS, UBE2C, and UNG; and/or
wherein the step of measuring the expression level of one or more gene products associated with proliferation comprises measuring the expression level of one, two or all three of MYBL2, TOP2A, and/or UBE2C gene products; and/or
wherein each of the gene products associated with cell adhesion impact at least two of an adherens junction, an anchoring junction, a cell-substrate adherens junction, a cell-substrate junction or a focal adhesion of a cell; and/or
wherein the step of measuring the expression level of at least one gene product associated with cell adhesion comprises measuring the expression level of a gene product of at least one gene selected from the group consisting of ATP2A2, BAIAP2, CD151, CHP1, CYFIP1, CYTH3, DAG1, DSC2, GIT1, HSP90B1, HSPA5, LIMK1, MAPK1, PACSIN2, PDIA3, PVR, REXO2, RPL22, RPLP1, RPLP2, RPS11, RPS16, RPS5, SDCBP SNAP23, SNTB1, and SRP68, optionally,
wherein the step of measuring the expression level of at least one gene product associated with cell adhesion comprises measuring the expression of a PVR gene product, optionally, wherein the step of measuring the expression level of at least one gene product associated with cell adhesion consists of measuring the expression level of a single gene product associated with cell adhesion, optionally,
wherein the single gene product is a PVR gene product or is not a PVR gene product; and/or
wherein measuring the expression level of at least one gene product associated with cell adhesion comprises measuring the expression level of two or more gene products associated with cell adhesion, and subsequently determining a cell adhesion gene product expression level by averaging the expression of two or more gene products associated with cell adhesion.

155. The method of claim 153, wherein the expression level of the at least one gene product associated with a corresponding ADC therapy is weighted by a factor of approximately 0.3 to 0.65, the expression level of at least one gene product associated with cell adhesion is weighted by a factor of approximately −0.8 to −1, and the determined proliferation gene expression level is weighted by a factor of approximately 0.2 to 0.4; or
wherein the expression level of the at least one gene product associated with a corresponding ADC therapy is weighted by a factor of approximately 0.3, the expression level of at least one gene product associated with cell adhesion is weighted by a factor of approximately −0.9, and the tumor cellularity is weighted by a factor of approximately 0.8, or
wherein the expression level of the at least one gene product associated with a corresponding ADC therapy is weighted by a factor of approximately 0.45, the expression level of at least one gene product associated with cell adhesion is weighted by a factor of approximately −1, the determined proliferation gene expression level is weighted by a factor of approximately 0.55, and the tumor cellularity is weighted by a factor of approximately 0.07; optionally,
wherein each of the one or more ADC TRS is determined by further taking into account a bias variable, wherein the bias variable is a static offset tuned to yield biomarker frequencies that match published objective response rates in clinical trials, optionally, wherein the bias variable is weighted by a factor of approximately −0.25.

156. The method of claim 153, wherein the predetermined threshold is set to a percentile of ranked ADC Treatment Response Scores determined from tumor tissue samples of a first cohort of subjects utilizing at least the expression level of the at least one gene product associated with a first corresponding ADC therapy, wherein each percentile corresponds to a percentage greater than a percentage of subjects of the first cohort which do not respond to the same first corresponding ADC therapy, optionally, wherein the first cohort of subjects is a pan-cancer cohort or a matched tumor-type; or
wherein the predetermined thresholds are set to percentiles of ranked ADC Treatment Response Scores determined from tumor tissue samples of a first and a second cohort of subjects utilizing at least the expression level of at least a first and a second gene product associated with a first and second corresponding ADC therapy, wherein each percentile corresponds to a percentage greater than a percentage of subjects of the first and second cohort which do not respond to the at least first and second corresponding ADC therapies, optionally, wherein the first and/or second cohort of subjects is a pan-cancer cohort or a matched tumor-type, optionally wherein the first cohort of subjects and the second cohort of subjects are the same cohort of subjects; or
wherein the predetermined threshold is zero, and an ADC TRS which indicates the subject is likely to respond to an ADC therapy is an ADC TRS with a value greater than zero.

157. The method of claim 153, wherein gene expression level of the at least one gene product associated with each one or more ADC therapies, the measured expression of the at least one gene product associated with cell adhesion, and the tumor cellularity are log 2 transformed and/or Z score normalized prior to step (e)(1), and the expression level of the one or more gene products associated with proliferation are log 2 transformed and/or Z score normalized prior to the step of averaging expression levels of gene products associated with proliferation to obtain the proliferation gene expression level in step (c); and/or
wherein the tumor cellularity is a molecularly determined tumor cellularity which is calculated based on somatic and germline variant allele frequencies and/or copy number profiles; and/or
wherein the expression product of the at least one gene associated with each of the one or more ADC therapies, the at least one gene associated with cell adhesion, and one or more genes associated with proliferation are individually selected from a ribonucleic acid (RNA) and a protein; and/or
wherein the gene expression product of the at least one gene associated with each of the one or more ADC therapies, the at least one gene associated with cell adhesion, and the one or more genes associated with proliferation are proteins, and measuring the expression level thereof requires making use of immunohistochemistry techniques; and/or
wherein the gene expression product of the at least one genes associated with each of the one or more ADC therapies, the at least one gene associated with cell adhesion, and the one or more genes associated with proliferation are RNA, and measuring the expression level thereof requires making use of RNA sequencing techniques or qPCR.

158. The method of claim 153, wherein each of the one or more ADC therapies comprise a monoclonal antibody, at least one functional fragment thereof or a bispecific antibody which targets at least one epitope of at least one antigen selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5, optionally, wherein the antibody, the at least one functional fragment thereof, or the bispecific antibody is either fused to a protein which is toxic to a cancer cell, or is conjugated directly or indirectly to a cytotoxic drug, optionally, wherein the cytotoxic drug is a DNA replication inhibitor selected from the group consisting of an alkylating agent, a DNA polymerase inhibitor, a nitrogen mustard and a topoisomerase inhibitor, optionally, wherein the cytotoxic drug is a topoisomerase inhibitor; or
wherein the antibody comprises a bispecific antibody capable of targeting two epitopes of the same antigen or an epitope of two distinct antigens, wherein the same antigen or the two antigens are selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5, optionally, wherein the bispecific antibody is fused to a protein which is toxic to a cancer cell, and/or is conjugated directly or indirectly to a cytotoxic drug, optionally, wherein the cytotoxic drug is a DNA replication inhibitor selected from the group consisting of an alkylating agent, a DNA polymerase inhibitor, a nitrogen mustard and a topoisomerase inhibitor, optionally, wherein the cytotoxic drug is a topoisomerase inhibitor; or
wherein the one or more ADC therapies comprise at least two therapies, and step (e)(1) comprises calculating at least two ADC Treatment Response Scores, wherein if the subject is identified likely to respond to at least two ADC therapies, the method further comprises step (e)(2) of ranking the at least two ADC Treatment Response Scores by the value by which each ADC Treatment Response Score exceeds the predetermined threshold, and identifying the highest ranked ADC therapy as the most likely to benefit the subject and in step (f) administering an effective amount of the highest ranked ADC therapy to the subject.

159. The method of claim 153, wherein the subject has or is suspected of having a cancer not approved for labeled use of the one or more ADC therapies; and/or
wherein the tumor tissue sample is or is suspected of containing bladder cancer, salivary gland cancer, endometrial cancer, ovarian cancer, cervical cancer, head and neck cancer, non-melanoma skin cancer, thyroid cancer, cancer of unknown primary, cancer of the central or peripheral nervous system, neuroendocrine tumor, melanoma, esophagogastric cancer, small bowel cancer, sarcoma, hepatobiliary cancer, pancreatic cancer, gastrointestinal stromal tumor, renal cell carcinoma, glioma, appendiceal cancer breast cancer, small cell lung cancer, prostate cancer, esophagogastric cancer, non-small cell lung cancer, pancreatic cancer, lymphoma or colorectal cancer, optionally,
wherein the tumor tissue sample is a formalin-fixed paraffin-embedded (FFPE) tumor tissue sample, optionally,
wherein the tumor tissue sample contains at least 20% tumor content.

160. A computer-implemented method for selecting a patient presenting with a solid cancerous tumor for treatment by one or more antibody-drug conjugate (ADC) therapies, wherein the method comprises the steps of:
(A) receiving a measured expression level of at least one gene product associated with each of the one or more ADC therapies from a biological tissue sample obtained from the tumor tissue of the patient;
(B)(1) receiving an expression level of one or both of:
(i) at least one gene product associated with cell adhesion, and
(ii) one or more gene products associated with proliferation,
wherein the expression levels of (i) and (ii) are measured in the same biological tumor tissue sample of step (A),
(B)(2) if the measured expression level of the one or more gene products associated with proliferation is received, calculating therefrom, with a computer, an average of all expression levels of the received measured gene products associated with proliferation, thereby obtaining a proliferation gene expression level;
(C) optionally, receiving an indication of tumor cellularity of the same tumor tissue sample of steps (A) and (B);
(D)(1) identifying a subject likely to respond to the one or more ADC therapies when either:
(i) one or more of a calculated ADC Treatment Response Score (ADC TRS) surpass one or more corresponding predetermined thresholds, wherein each of the one or more ADC TRS is determined from:
(1) the received measured expression level of the at least one gene product associated with a corresponding ADC therapy, and
at least two of:
(2) the received measured expression level of the at least one gene product associated with cell adhesion,
(3) the calculated proliferation gene expression level from the received expression levels of one or more gene products associated with proliferation, and
(4) the received indication of tumor cellularity; and
(E) selecting the patient identified as likely to respond to the one or more ADC therapies for treatment therewith,
wherein at least steps (A)-(D)(1)(i) are performed with a suitably programmed computer, optionally,
wherein step (E) comprises selecting the patient to receive treatment with the one or more ADC therapies as part of a clinical trial, optionally, wherein the clinical trial is a basket trial, optionally,
wherein the method further comprises the step (F) treating the selected patient with the one or more ADC therapies determined to likely induce a response in the patient.

161. The method of claim 160, wherein the ADC TRS is determined by at least the combination of the expression level of the at least one gene product associated with a corresponding ADC therapy, the expression level of the at least one gene product associated with cell adhesion, and the determined proliferation gene expression level; and/or
wherein both the determined proliferation gene expression level and the expression level of the at least one gene product associated with a corresponding ADC therapy are positively associated with a likelihood that a patient will benefit from the corresponding ADC therapy, and the measured expression level of the at least one gene product associated with cell adhesion is negatively associated with a likelihood that a patient will benefit from the same corresponding ADC therapy; and/or
wherein the ADC TRS is determined by at least the combination of the expression level of the at least one gene product associated with a corresponding ADC therapy, the measured expression level of the at least one gene product associated with cell adhesion, and tumor cellularity; and/or
wherein both the tumor cellularity and the expression level of the at least one gene product associated with a corresponding ADC therapy are positively associated with a likelihood that a patient will benefit from the corresponding ADC therapy, and the measured expression level of the at least one gene product associated with cell adhesion is negatively associated with a likelihood that a patient will benefit from the same corresponding ADC therapy; and/or
wherein the ADC TRS is determined by at least the combination of the expression level of the at least one gene product associated with a corresponding ADC therapy, the measured expression level of the at least one gene product associated with cell adhesion, the determined proliferation gene expression level, and tumor cellularity; and/or
wherein each of the determined proliferation gene expression level, the tumor cellularity, and the expression level of the at least one gene product associated with a corresponding ADC therapy are positively associated with a likelihood that a patient will benefit from the corresponding ADC therapy, and the measured expression level of the at least one gene product associated with cell adhesion is negatively associated with a likelihood that a patient will benefit from the same corresponding ADC therapy.

162. The method of claim 160, wherein each of the one or more ADC TRS is determined by further taking into account a bias variable, wherein the bias variable is a static offset tuned to yield biomarker frequencies that match published objective response rates in clinical trials; and/or
wherein the predetermined threshold is set to a percentile of ranked ADC Treatment Response Scores determined from tumor tissue samples of a first cohort of subjects utilizing at least the expression level of the at least one gene product associated with a first corresponding ADC therapy, wherein each percentile corresponds to a percentage greater than a percentage of subjects of the first cohort which do not respond to the same first corresponding ADC therapy, optionally,
wherein the first cohort of subjects is a pan-cancer cohort or a matched tumor-type cohort; and/or
wherein the predetermined threshold is set at zero, and an ADC TRS which indicates the subject is likely to benefit from an ADC therapy is an ADC TRS with a value greater than zero; and/or
wherein gene expression level of the at least one gene product associated with each one or more ADC therapies, the measured expression level of the at least one gene product associated with cell adhesion, and optionally the tumor cellularity are log 2 transformed and/or Z score normalized prior to step (D)(1), and the expression level of the one or more gene products associated with proliferation are log 2 transformed and/or Z score normalized prior to the step of averaging expression levels of gene products associated with proliferation to obtain the proliferation gene expression level in step (B)(2); and/or
wherein the tumor cellularity is a molecularly determined tumor cellularity which is calculated based on somatic and germline variant allele frequencies and/or copy number profiles.

163. The method of claim 160, wherein the expression product of the at least one gene associated with each of the one or more ADC therapies, the at least one gene associated with cell adhesion, and one or more genes associated with proliferation are individually selected from a ribonucleic acid (RNA) and a protein; or
wherein the gene expression product of the at least one gene associated with each of the one or more ADC therapies, the at least one gene associated with cell adhesion, and the one or more genes associated with proliferation are proteins, and measuring the expression level thereof requires making use of immunohistochemistry techniques; or
wherein the gene expression product of the at least one genes associated with each of the one or more ADC therapies, the at least one gene associated with cell adhesion, and the one or more genes associated with proliferation are RNA, and measuring the expression level thereof requires making use of RNA sequencing techniques or qPCR.

164. The method of claim 160, wherein each of the one or more ADC therapies comprise a monoclonal antibody, at least one functional fragment thereof or a bispecific antibody which targets at least one epitope of at least one antigen selected from the group consisting of SLC39A6, VTCN1, ERBB3, MET, ERBB2, TACSTD2, FOLR1, PVRL4, F3, SLC34A2 MSLN, B7-H3, B7-H4, 5T4, GPR20, AXL, TFR1, P79, EGFR, Integrin beta-6, ROR1, Globo H, IL2RA, GCC, MCP, FLT3, PTK7, IGF-1R, AG7, NCAM1, LY75, ALCAM, and CEACAM5, optionally, wherein the antibody, the at least one functional fragment thereof, or the bispecific antibody is conjugated directly or indirectly to a cytotoxic drug or is fused to a cytotoxic protein; and/or
wherein the subject has or is suspected of having a cancer not approved for labeled use of the one or more ADC therapies; and/or
wherein the tumor tissue sample is or is suspected of containing bladder cancer, salivary gland cancer, endometrial cancer, ovarian cancer, cervical cancer, head and neck cancer, non-melanoma skin cancer, thyroid cancer, cancer of unknown primary, cancer of the central or peripheral nervous system, neuroendocrine tumor, melanoma, esophagogastric cancer, small bowel cancer, sarcoma, hepatobiliary cancer, pancreatic cancer, gastrointestinal stromal tumor, renal cell carcinoma, glioma, appendiceal cancer breast cancer, small cell lung cancer, prostate cancer, esophagogastric cancer, non-small cell lung cancer, pancreatic cancer, lymphoma or colorectal cancer, optionally,
wherein the tumor tissue sample is a formalin-fixed paraffin-embedded (FFPE) tumor tissue sample, optionally,
wherein the tumor tissue sample contains at least 20% tumor content.

165. A method of identifying a subject as likely to benefit from anti-TROP2 based therapy, comprising:
(a) measuring the expression level of a TROP2 gene product and one or more gene products associated with proliferation in a tumor tissue sample obtained from the subject;
(b) measuring the expression levels of one or more housekeeping genes in the same tumor tissue sample of step (a), wherein the one or more housekeeping genes comprise three genes selected from CIAO1, EIF2B1, HMBS, CTCF, GGNBP2, ITGB7, MYC and SLC4A1AP, and further normalizing the expression levels of the TROP2 gene product and one or more gene products associated with proliferation of step (a) to the three housekeeping genes to obtain normalized expression levels of the TROP2 and one or more genes associated with proliferation;
(c) determining proliferation gene expression level by averaging the normalized expression levels of the one or more gene products associated with proliferation;
(d) determining tumor cellularity in the same tumor tissue sample of step (a); and
(e) identifying the subject as likely to benefit from the anti-TROP2 based therapy when either i) an aggregate biomarker score surpasses a predetermined threshold, wherein the aggregate biomarker score is calculated from the combination of the measured expression level of the TROP2 gene product and at least one of the determined proliferation gene expression level and/or the determined tumor cellularity, or when ii) the measured expression level of the TROP2 gene product is higher than a median TROP2 expression level obtained from tumor tissue samples from a first cohort of subjects, and at least one of the determined proliferation gene expression level and/or the determined tumor cellularity is higher than a median proliferation gene expression level and/or a median tumor cellularity obtained from tumor tissue samples from the same first cohort of subjects, optionally,
wherein the subject is identified as likely to benefit from the anti-TROP2 based therapy, when the measured expression level of the TROP2 gene product, the determined proliferation gene expression level, and the determined tumor cellularity are all higher than corresponding median levels of TROP2 expression, proliferation gene expression and median tumor cellularity obtained from tumor tissue samples from the same first cohort of subjects, optionally, wherein the first cohort of subjects is a pan-cancer cohort or a matched tumor-type cohort; and/or
wherein the subject is identified as likely to benefit from the anti-TROP2 based therapy when one or more of the measured expression levels of the TROP2 gene product, the determined proliferation gene expression level, and/or the determined tumor cellularity fall into the highest quartile of TROP2 expression level, proliferation gene expression level and/or tumor cellularity values obtained from tumor tissue samples from the same first cohort of subjects, optionally, wherein the first cohort of subjects is a pan-cancer cohort or a matched tumor-type cohort; and/or
wherein measuring the expression level of one or more gene products associated with proliferation comprises measuring the expression levels of gene products of one or more genes selected from the group consisting of BIRC5, BRCA1, BRCA2, BUB1, BUB1B, CCNA2, CCNB1, CCNB2, CCNE1, CCNE2, CCNF, CCNG2, CDC2, CDC20, CDC25A, CDC25B, CDC25C, CDC45L, CDC6, CDC7, CDCKN1A p21, CDCKN3, CDK4, CDKN1C, CDKN2A, CDKN2C, CDKN2D p19, CDKN3, CENPA, CENPE, CENPF, CHAF1A, CHK1, CKS1, CKS2, CKS2, DHFR, DHFR, E2-EPF, E2F1, E2F3, E2F5, FEN1, FOXM1, KNSL2, KNSL5, KPNA2, LMNB2, MAD2, MAD2L1, MAPK13, MCM2, MCM3, MCM4, MCM5, MCM6, MK167, MNAT1, MSH2, MYBL2, NASP, NEK2, NPAT, ORC1, PA2G4, PCNA, PES1, PKMYT1, PLK, PRIM1, PTTG1, RAB6KIFL, RACGAP1, RAD51, RAD54L, RAN RAN, RFC4, RPA3, RRM1, RRM2, SLBP, STK12, STK15, STK18, STK6, TOP2A, TROAP, TUBB, TYMS, UBE2C, and UNG; and/or
wherein measuring the expression level of one or more gene products associated with proliferation comprises measuring the expression level of one, two or all three of MYBL2, TOP2A, and/or UBE2C gene products; and/or
wherein the aggregate biomarker score is determined by the combination of the expression level of TROP2 gene products, the determined proliferation gene expression level, and tumor cellularity; and/or
wherein the predetermined threshold is set to the percentile of ranked aggregate biomarker scores determined from tumor tissue samples of a second cohort of subjects, which percentile corresponds to a percentage of subjects of the second cohort which do not respond to an anti-TROP2 based therapy, optionally, wherein the second cohort of subjects is a pan-cancer cohort or a matched tumor-type cohort, optionally, wherein the first cohort of subjects and the second cohort of subjects are the same cohort of subjects; and/or
wherein gene expression levels of TROP2, one or more genes associated with proliferation, and tumor cellularity are log 2 transformed and/or median-centered to 10 prior to step (d); and/or
wherein the tumor cellularity is a molecularly determined tumor cellularity which is calculated based on somatic and germline variant allele frequencies and/or copy number profiles; and/or
wherein one, two or three of the housekeeping genes are selected from CIAO1, EIF2B1, and HMBS; and/or
wherein the aggregate biomarker score is determined by adding the measured expression level of TROP2 gene product to approximately ⅓ to ⅔ the determined proliferation gene expression level and approximately 4-8 times the determined tumor cellularity; and/or
wherein the expression products of TROP2 and one or more genes associated with proliferation are individually selected from a ribonucleic acid (RNA) and a protein; and/or
wherein the gene expression product of at least one of TROP2 and one or more genes associated with proliferation is a protein, and measuring the expression level thereof requires making use of immunohistochemistry techniques; and/or
wherein the gene expression product of at least one of TROP2 and one or more genes associated with proliferation is a RNA, and measuring the expression level thereof requires making use of RNA sequencing techniques or qPCR.

166. The method of claim 165, wherein the anti-TROP2 based therapy, comprises an anti-TROP2 antibody or fragment thereof, optionally,
wherein the anti-TROP2 antibody or fragment thereof is conjugated directly or indirectly to a cytotoxic drug or is fused to a protein which is toxic to a cancer cell, optionally,
wherein the cytotoxic drug is a DNA replication inhibitor selected from the group consisting of an alkylating agent, a DNA polymerase inhibitor, a nitrogen mustard and a topoisomerase inhibitor, optionally, wherein the cytotoxic drug is a topoisomerase inhibitor; optionally, wherein the anti-TROP2 based therapy is Sacituzumab govitecan; and/or
wherein the subject has or is suspected of having a cancer not approved for labeled use of anti-TROP2 based therapy; and/or
wherein the tumor tissue sample is or is suspected of containing bladder cancer, endometrial cancer, breast cancer, small cell lung cancer, prostate cancer, esophagogastric cancer, non-small cell lung cancer, pancreatic cancer, or colorectal cancer, optionally,
wherein the tumor tissue sample is a formalin-fixed paraffin-embedded (FFPE) tumor tissue sample, optionally,
wherein the tumor tissue sample contains at least 20% tumor content; and/or
wherein the method further comprises administering an anti-TROP2 based therapy to the subject identified as likely to benefit from the anti-TROP2 based therapy.
